(19) United States
(12) Patent Application Publication
(10) Pub. No.: US 2013/0005593 A1
(43) Pub. Date: Jan. 3, 2013

(54) RNA-Oligonucleotide Quantification Technique for the Enumeration of Uncultivated Bacterial Species

(76) Inventor: Flavia R. F. Teles, Brookline, MA (US)

(21) Appl. No.: 13/416,751

(22) Filed: Mar. 9, 2012

Related U.S. Application Data
(60) Provisional application No. 61/450,894, filed on Mar. 9, 2011.

(51) Int. Cl. C40B 30/04; G01N 27/72; G01N 21/78; G01N 21/76; C12Q 1/68; G01N 21/64

(52) U.S. Cl. 506/9; 435/6.11

(57) ABSTRACT

Methods for RNA-Oligonucleotide Quantification Technique for the Enumeration of Uncultivated Bacterial Species are disclosed.

### RELATED APPLICATION

This application claims the benefit of U.S. Provisional Application No. 61/450,894, filed Mar. 9, 2011.

The entire teachings of the above application are incorporated herein by reference.

### GOVERNMENT SUPPORT

The invention was supported, in whole or in part, by grants T32-DE-07327, DE-12108 and R03 DE021742 from the National Institutes of Dental and Craniofacial Research. The Government has certain rights in the invention.

### BACKGROUND OF THE INVENTION

Periodontal diseases are polymicrobial infections that can lead to periodontal inflammation, alveolar bone and tooth loss. One key objective in studying these diseases is the discrimination of host-compatible and pathogenic species. Certain cultivated species have been determined to be periodontal pathogens in part because increased levels of these species were associated with periodontitis and their levels decreased after successful periodontal therapy.

The oral cavity harbors more than 700 taxa, 35% of which have not yet been cultivated. It is likely that the uncultivated segment of the microbiota harbors pathogenic as well as beneficial species. Certain oral uncultivated taxa have been “discovered” by cloning and further investigated using PCR-based techniques, in situ hybridization and more recently microarrays and next generation sequencing. However, none of these techniques have the ability to quantify the levels of multiple uncultivated species in large numbers of individual samples simultaneously. Absolute numbers and proportions of organisms in multiple individual samples are important in distinguishing species associated with periodontal health or disease and to evaluate the effects of periodontal therapy.

When attempting to distinguish possible periodontal pathogens from non-pathogenic “uncultivable” species, one will likely have to examine a wide range of candidate taxa in a large number of subgingival biofilm samples from various states of periodontal health or disease. This is due to the rather large variability encountered in the microbial composition of subgingival biofilm samples. Thus, a need exists to develop a high throughput method to quantify a wide range of uncultivable and cultivable taxa in large numbers of subgingival biofilm samples.

### SUMMARY OF THE INVENTION

The present invention relates to methods for quantifying one or more microorganisms in a sample from an individual. The method involves extracting the total nucleic acid (TNA) from the sample; contacting TNA from the sample with one or more probes that correlate to a microorganism to be quantified, under conditions suitable for hybridization to thereby form a complex or hybrid; and detecting the amount of a complex or hybrid in the sample by comparing the hybridization signals with known amounts of hybrids. The amount of the hybrids correlates with the amount of TNA of one or more microorganism in the sample. Based on standards for quantification, levels of bacterial cells from that microorganism can be determined from the amounts of TNA. Cultivated microorganisms, uncultivated microorganisms, or both can be quantified. In an embodiment, a plurality or number of microorganisms is quantified at once e.g., in a high throughput screening. The methods can employ detection 16S rRNA of the microorganism. Samples used in the methods described herein can come from the oral cavity, sinus, esophagus, respiratory tract, lungs, sputum, pharynx, eustachian tube, middle ear, vagina, blood, pus, spinal fluid, gastrointestinal tract, or combination thereof. The methods, in an aspect, further involve labeling oligonucleotide sequences that identify specific taxa using detectable label (e.g., digoxigenin, fluorescent dyes, streptavidin conjugate, magnetic beads, dendrimers, radiolabels, enzymes, colorimetric labels, nanoparticles, and nanocrystals). The TNAs used in the methods of the present invention can be bound to a solid support, such as nylon membrane, glass, silica chips, polymer, plastic, ceramic, metal, optical fiber or any combination thereof.

The present invention further pertains to methods for identifying new microbial pathogens, assisting in the diagnosis of an individual having a disease or condition and monitoring the effects of treatment of diseases. The steps of the method relates to determining the amount of TNA of one or more cultivated or uncultivated microorganisms in a sample from the individual, wherein an amount of TNA of a microorganism correlates to a disease state or a healthy state. An amount of TNA between about 10³ and about 10⁶ of a given taxon correlates to a disease state or a healthy state, and an amount less than 10³ indicates health, depending on the nature of the taxon.

Yet another embodiment of the present invention involves methods for diagnosing an individual having a periodontal disease. This method includes the step of determining the amount of TNA of one or more cultivated or uncultivated microorganisms in a sample from the individual. An amount of TNA between about 10³ and about 10⁶ of one or more of the following microorganism correlates to a disease state or a healthy state, and an amount less than 10³ does not correlate to a disease state or a healthy state: Fusobacterium nucleatum. ss polymorphum, Actinomyces gerencseriae, Mitsuokella sp OT 131, Prevotella sp OT_{—}306, Porphyromonas gingivalis, Peptostreptococcus strains BS404 or CK035, Desulfobulbus sp OT 041, Synergistetes_OT_{—359}, Selenomonas_OT_{—}134_{—}442, TM7_OT_{—}346_{—}349, Capnocytophaga_OT_{—}335, Haemophilus_OT_{—}035, Synergistetes_OT_{—}363_{—}453_{—}452, Treponema_OT_{—}256_{—}508_{—}517, Actinomyces_OT_{—}177 and Bacteroidetes_OT_{—}274. Throughout this document OT or oral taxon designations for uncultivated/unrecognized taxa are provided in accord with Human Oral Microbiome Database (HOMD, www.homd.org). The probes for the organisms listed herein are shown in FIGS. 6A-6R and HOMD is described in corresponding U.S. patent application Ser. No. 11/556,296, entitled “Methods and Arrays For Identifying Human Microflora” the entire teachings of which are incorporated herein by reference).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A and FIG. 1B are photographs of a nylon membrane showing RNA-Oligonucleotide Quantification Technique (ROQT) membrane used to assess probe sensitivity. TNAs extracted from selected bacterial species were used as targets in the horizontal lanes. Oligonucleotide probes in the vertical lanes were hybridized against these targets; a) Targets at 10⁶-10³ bacterial cells. Due to higher than ideal concentration of the Campylobacter rectus probe, signals became saturated for both cell concentrations. b) Targets at 10⁵-10⁴ bacterial cells.

FIG. 2 is a photograph of a nylon membrane showing the assessment of probe specificity. TNAs from selected bacterial species were used as targets in the horizontal lanes. Probes to the same species were hybridized against these targets in the vertical lanes. This figure shows 9 of the 23 target taxa tested.

FIG. 3 is a photograph of a nylon membrane showing the effects of different levels of species in mixtures on signal intensity. A a.: Aggregatibacter actinomycetemcomitans, A.o.: Actinomyces odontolyticus, C.r.: Campylobacter rectus, C.s.: Capnocytophaga sputigena, P.e.: Porphyromonas endodontalis, P.m.: Parvimonas micra, S.p.: Streptococcus parasanguinis.

FIG. 4 is a photograph of a checkerboard membrane showing hybridization of clinical samples with oligonucleotide probes. Probes for cultivated species (black) and as yet uncultivated species (green) are listed across the top. Each horizontal lane represents the total nucleic acids (TNA) from a sample from the indicated numbered tooth. Standards comprised a mixture of “complementary” sequences from all the test taxa at 0.004 and 0.04 pM, respectively. Teeth marked with an asterisk (*) were absent.

FIG. 5a is a graph wherein the mean estimated “counts” (×10⁵±SEM) of 20 bacterial taxa in 266 subgingival plaque samples obtained from 8 periodontally healthy and 11 periodontitis subjects. The species were ordered according to mean counts. Taxa in black represent cultivated species, while those in orange represent uncultivated taxa.

FIG. 5b is a graph wherein the mean estimated “counts” (×10⁵±SEM) of 20 bacterial taxa in 266 samples obtained from 8 periodontally healthy (green) and 11 periodontitis subjects (red). The species were ordered according to mean counts in health. Taxa in black represent cultivated species, while those in orange represent uncultivated taxa.

FIGS. 6A-6R include a table showing a list of oligonucleotide sequences that can be used to identify the corresponding microorganism and the immediate flanking sequence of the probes and the corresponding microorganism that is identified by that probe. Specifically, the microorganism (probe target) Genbank Accession number, Probe ID, Probe Sequence, and Probe Sequence Extended (probe with flanking regions), and sequence identifier.

### DETAILED DESCRIPTION OF THE INVENTION

A description of preferred embodiments of the invention follows.

The present invention relates to methods and systems for determining the amount of Total Nucleic Acids (TNA) in a sample and relate that to levels of bacterial cells in a given sample. In particular, the present invention pertains to determining the TNA for uncultivated bacteria in a sample (and therefore their levels) and using these quantities to determine the state of the tissue (e.g., disease or healthy) from which the sample was taken.

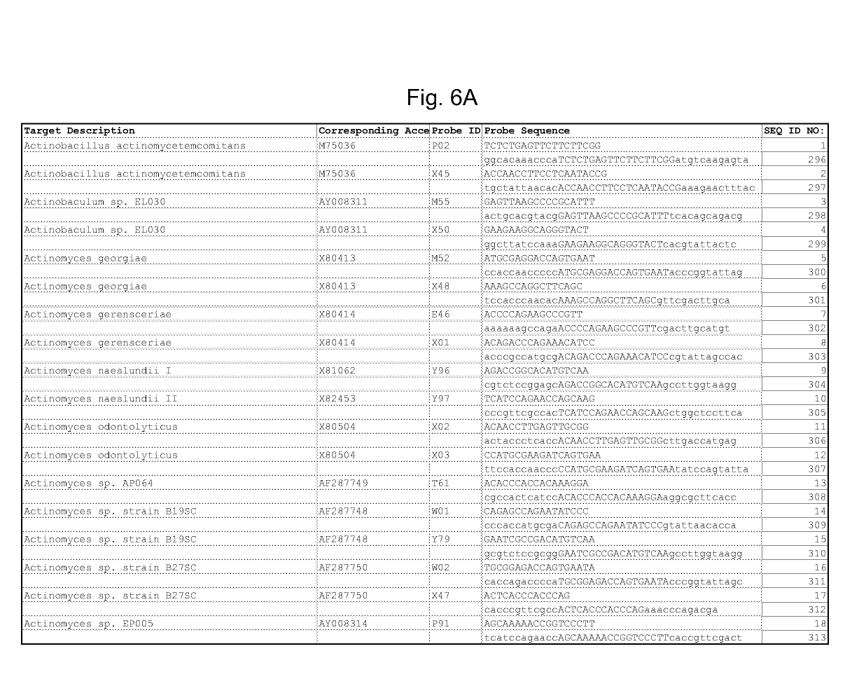

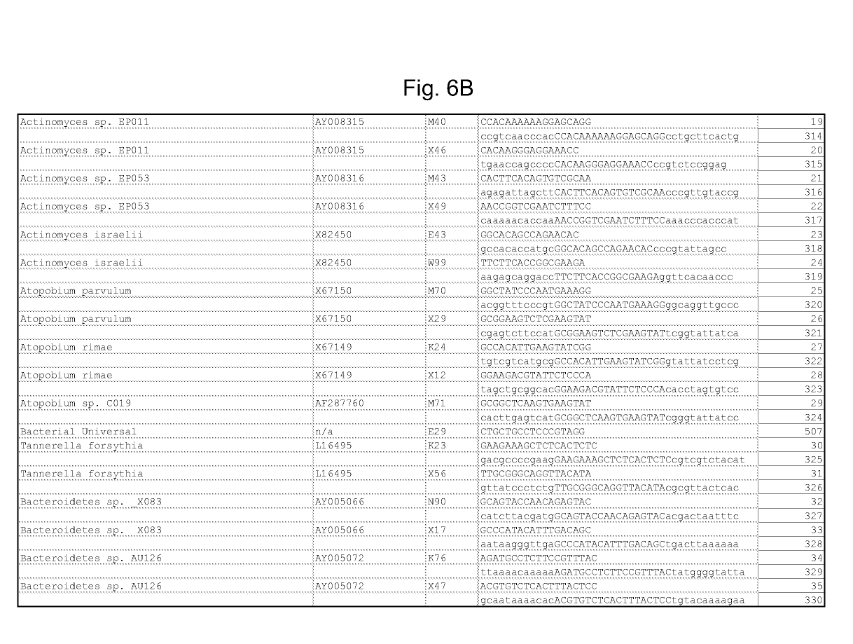

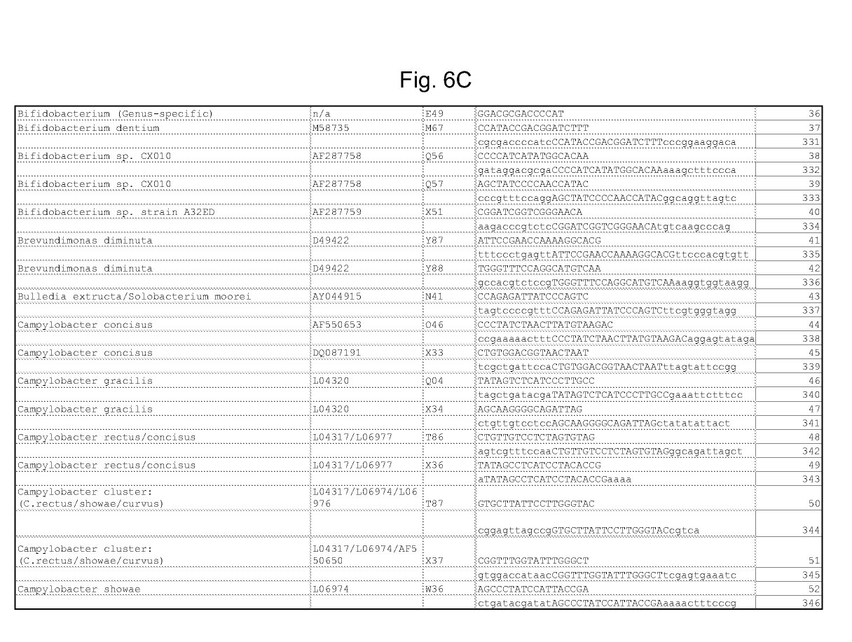

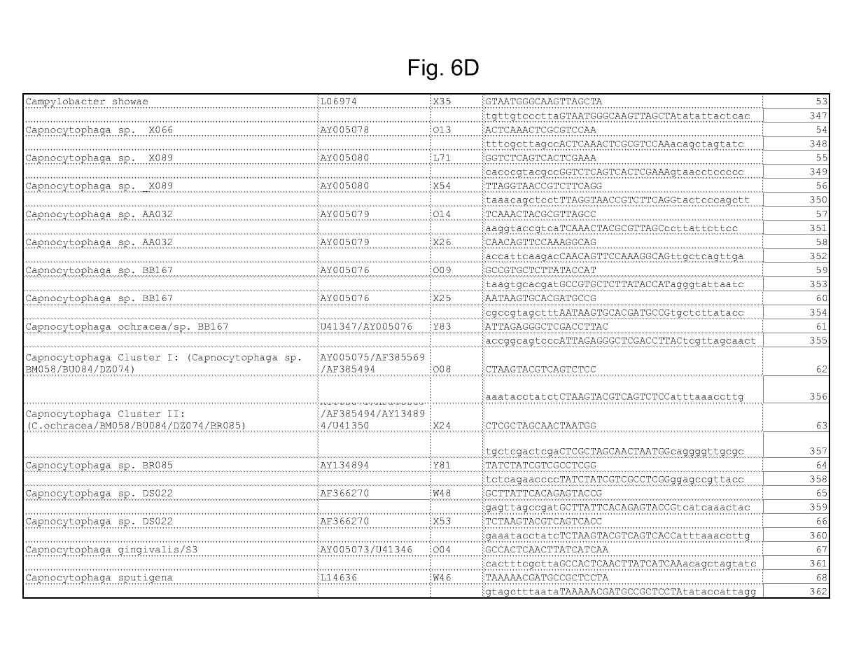

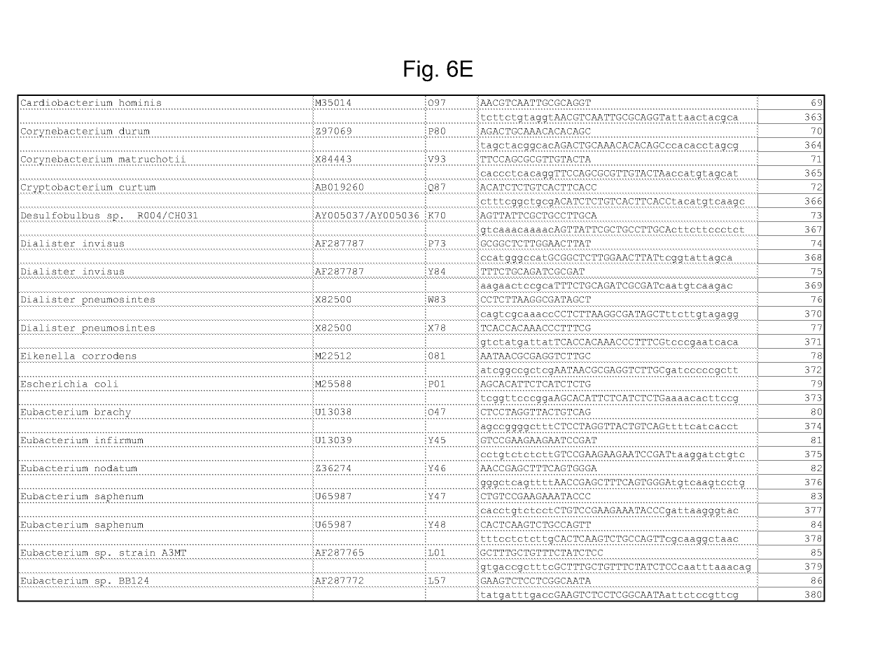

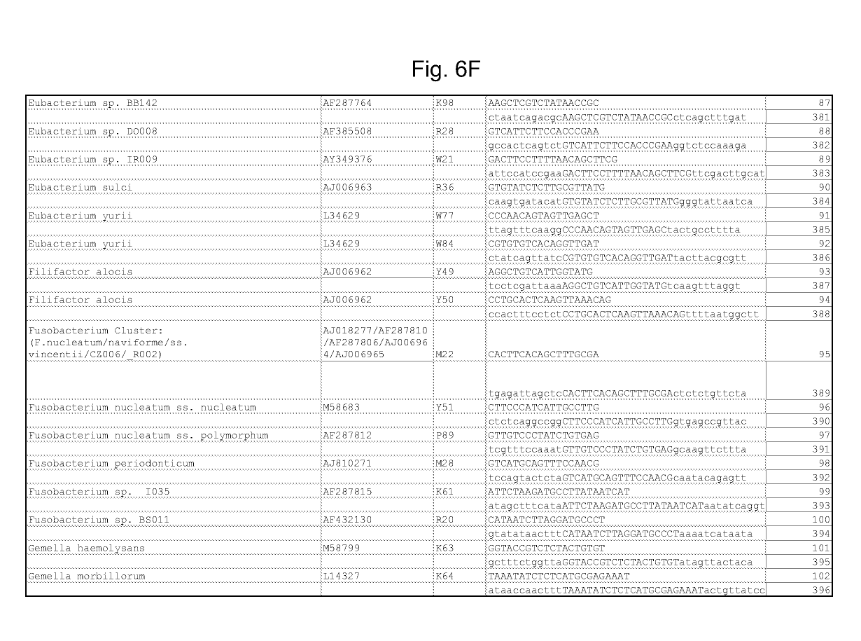

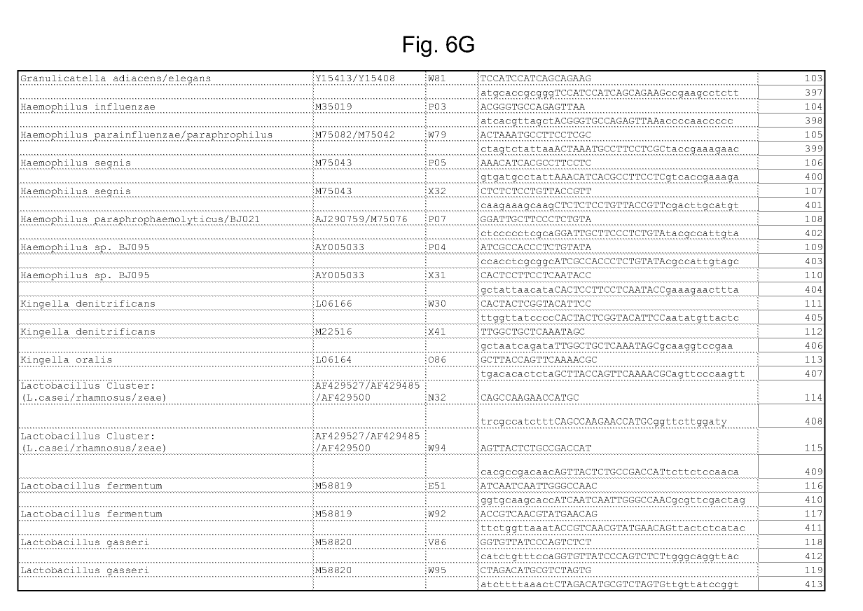

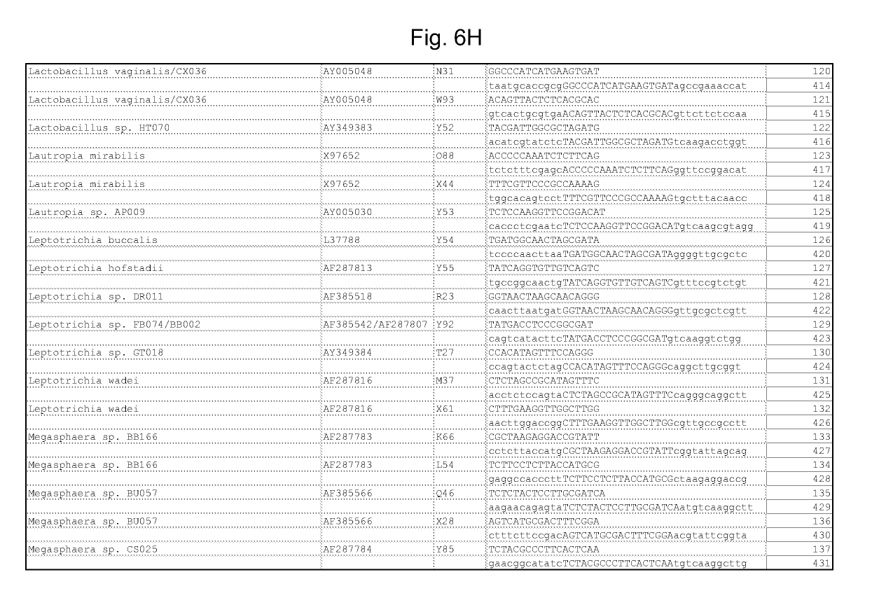

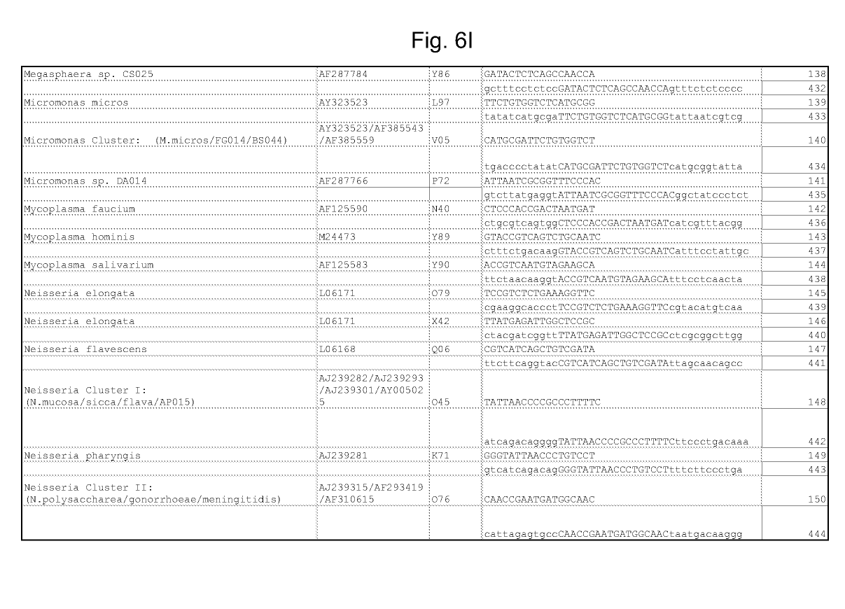

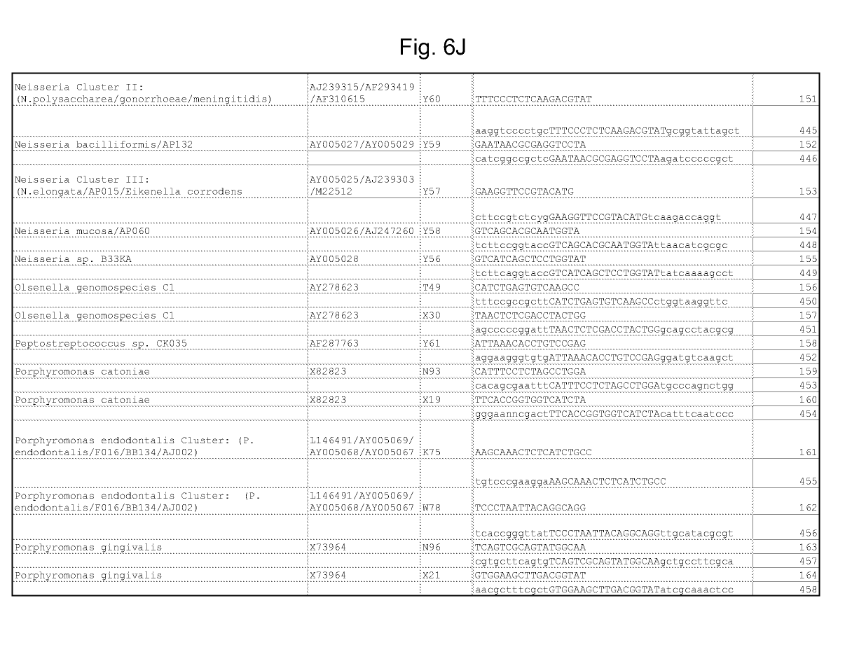

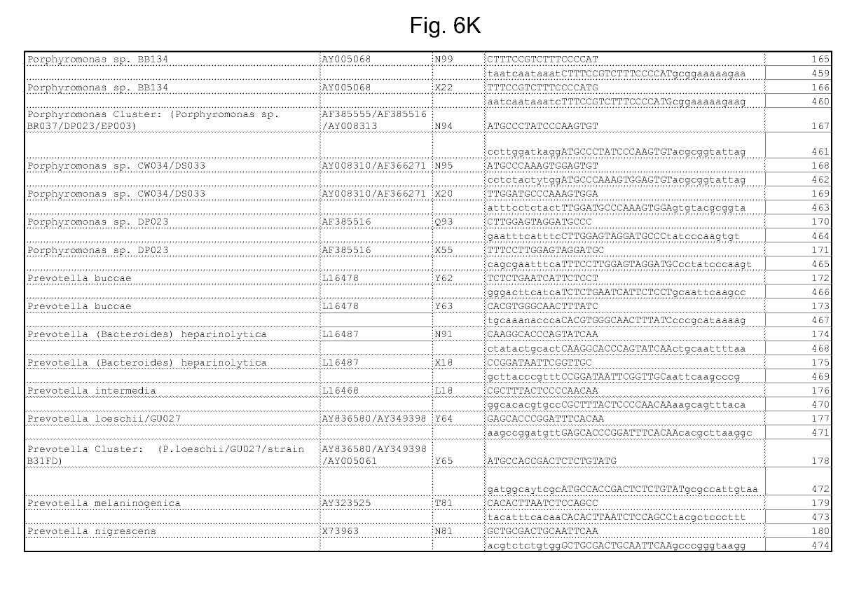

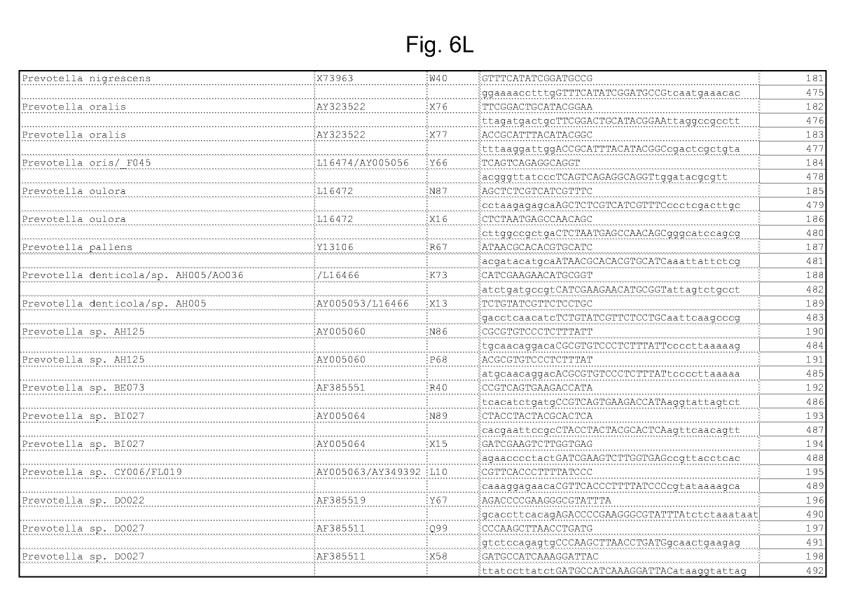

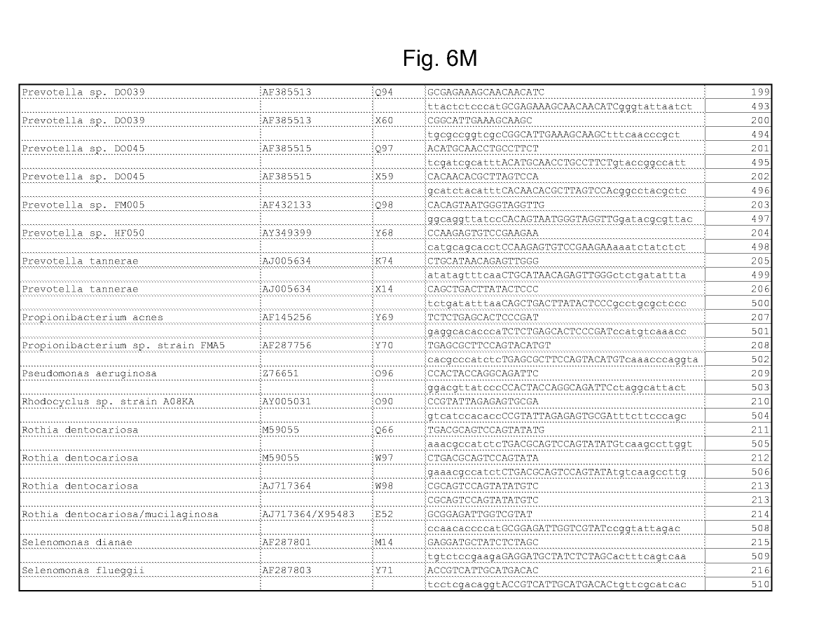

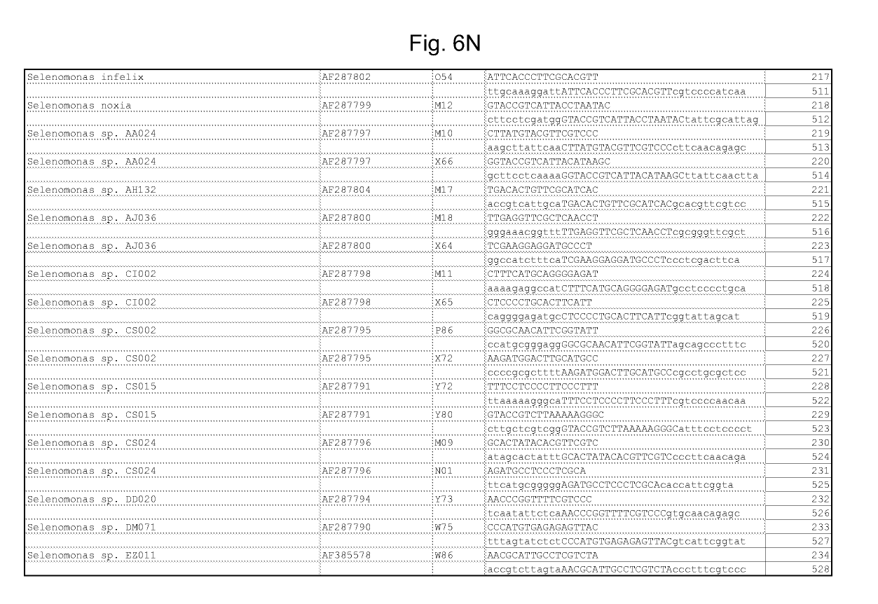

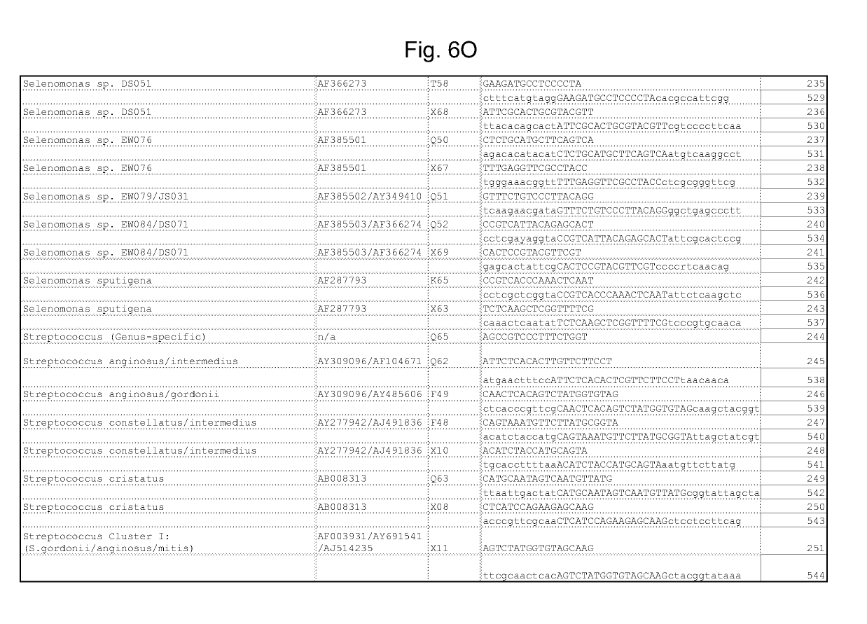

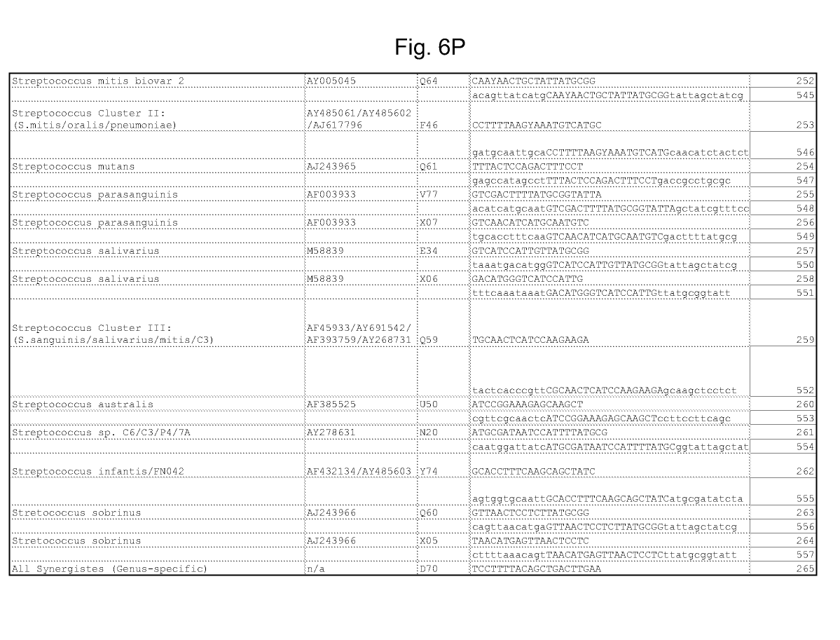

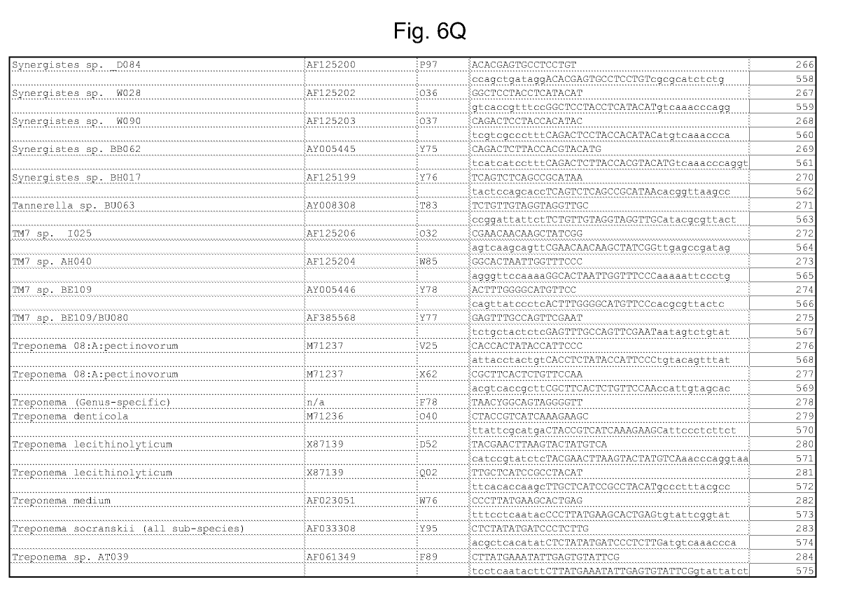

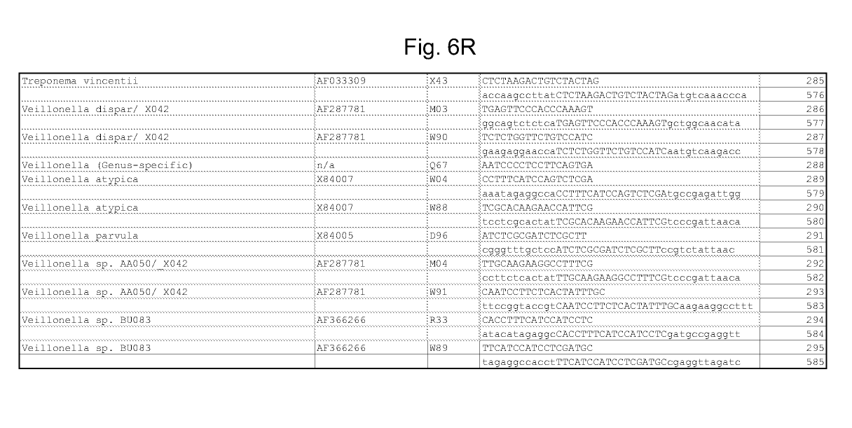

The role of uncultivated species in any number of pathogenesis is not known. Studying and quantifying uncultivated bacterial species can shed light into the pathogenesis of certain diseases. For example, approximately 35% of the species present in subgingival biofilms are as yet uncultivated, thus their role in periodontal pathogenesis is unknown. The methods described herein include a high throughput method to quantify a wide range of cultivated and uncultivated taxa in subgingival biofilm samples associated with periodontal disease or health. Oligonucleotides targeting the 16S rDNA gene were designed, synthesized and labeled with digoxigenin. See FIG. 6 for a list of probes. The steps of the method include hybridizing these probes with the total nucleic acids (TNA) of pure cultures or subgingival biofilm samples. Target species included cultivated taxa associated with periodontal health and disease, as well as uncultivated species, such as TM7 sp OT 346, Mitsuokella sp OT 131, and Desulfobulbus sp OT 041. Sensitivity and specificity of the probes were determined. A universal probe was used to assess total bacterial load. Sequences complementary to the probes were used as standards for quantification. Chemiluminescent signals were visualized after film exposure or using a CCD camera. In a clinical study, 266 subgingival plaque samples from 8 periodontally healthy and 11 periodontitis subjects were examined. Probes were specific and sensitivity reached 10⁴ cells. Fusobacterium nucleatum ss polymorphum and Actinomyces gerencseriae were the most abundant cultivated taxa in clinical samples. Among uncultivated/unrecognized species, Mitsuokella sp OT 131 and Prevotella sp OT 306 were the most numerous. Porphyromonas gingivalis and Desulfobulbus sp OT 041 were only detected in periodontitis patients. Direct hybridization of TNA using oligonucleotide probes permitted the quantification of multiple cultivated and uncultivated taxa in mixed species biofilm samples.

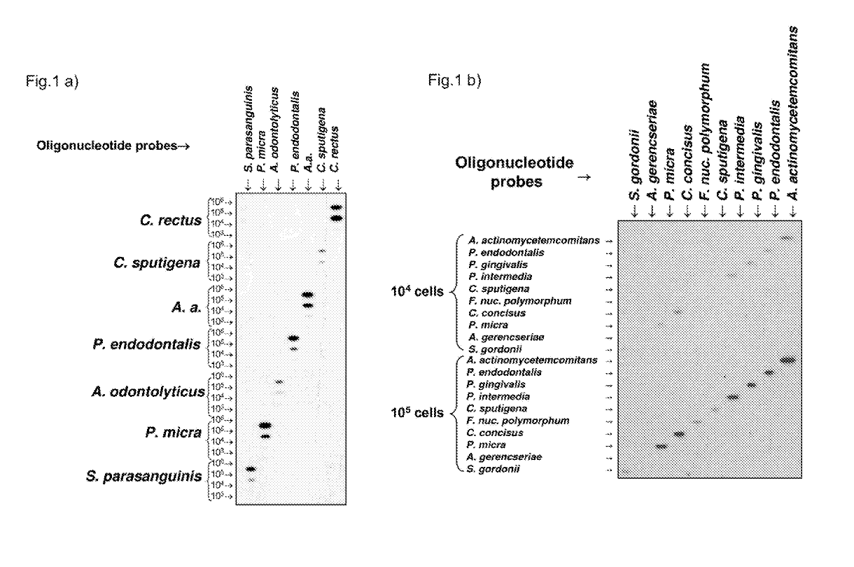

FIG. 1 demonstrates the checkerboard format of the assay as well as the sensitivity of detection of the oligonucleotide probes. For all the species tested, signals could be detected on film at the level of 10⁵ bacterial cells and for most species at 10⁴ cells, using a CCD camera.

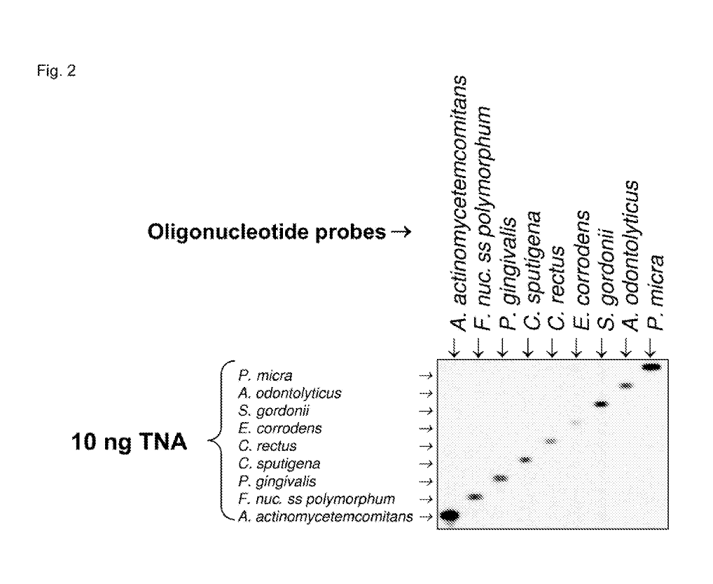

Probes for cultivated species were tested for specificity against cultivated species, using 10 ng of TNA as target (FIG. 2). Due to the unavailability of cells from uncultivated taxa, probes from those species were tested against their complementary oligonucleotide sequences. These probes were also tested against TNAs extracted from 96 cultivated bacterial species (Table 1). When a signal provided by the hybridization of a test probe with the TNA from any of those species was greater than 10% of the signal provided by the Universal probe for the same TNA, the probe was eliminated from the panel. This was the case for probes for Haemophilus sp oral taxon (OT) 035, Selenomonas sp OT 134, and Synergistetes sp OT 363. None of the probes for the

TABLE 1
Reference bacterial strains used to test specificity of oligonucleotide probes
and their American Type Culture Collection (ATCC) numbers.

| | |
|---|---|
| Aggregatibacter actinomycetemcomitans ** | Lactobacillus salivarius (11741) |
| Actinomyces georgiae (49285) | Lactobacillus oris (49062) |
| Actinomyces gerencseriae (23860) | Legionella pneumophila (33153) |
| Actinomyces israelii (12102) | Leptotrichia buccalis (14201) |
| Actinomyces meyeri (35568) | Neisseria mucosa (19696) |
| Actinomyces naeslundii I (12104) | Olsenella uli (49627) |
| Actinomyces naeslundii II (43146) | Parvimonas micra (33270) |
| Actinomyces odontolyticus (17929) | Peptostreptococcus anaerobius (27337) |
| Atopobium rimae (49626) | Porphyromonas endodontalis (35406) |
| Bacteroides fragilis (25285) | Porphyromonas gingivalis (33277) |
| Campylobacter concisus (33237) | Prevotella denticola (35308) |
| Campylobacter curvus 35224 | Prevotella intermedia (25611) |
| Campylobacter gracilis (33236) | Prevotella loescheii (15930) |
| Campylobacter rectus (33238) | Prevotella melaninogenica (25845) |
| Campylobacter showae (51146) | Prevotella nigrescens (33563) |
| Campylobacter sputorum ss sputorum (35980) | Prevotella oris (33573) |
| Campylobacter ureolyticus (33387) | Prevotella tannerae (51259) |
| Capnocytophaga gingivalis (33624) | Propionibacterium acnes I *** |
| Capnocytophaga ochracea (33596) | Propionibacterium acnes II *** |
| Capnocytophaga sputigena (33612) | Propionibacterium propionicum (14157) |
| Dialister invisus (GBA27) | Rothia dentocariosa (17931) |
| Eikenella corrodens (23834) | Selenomonas artemidis (43528) |
| Enterococcus aerogenes (13048) | Selenomonas infelix (43523) |
| Enterobacter sakazaki (12868) | Selenomonas noxia (43541) |
| Enterococcus faecalis (10100 & 29212) | Selenomonas sputigena (35185) |
| Escherichia coli (10799) | Slackia exigua (700122) |
| Eubacterium brachy (33089) | Staphylococcus aureus (33591) |
| Eubacterium limosum (8486) | Staphylococcus epidermidis (14990) |
| Eubacterium nodatum (33099) | Staphylococcus warneri (27836) |
| Eubacterium saburreum (33271) | Streptococcus anginosus (33397) |
| Eubacterium saphenum (49989) | Streptococcus constellatus (27823) |
| Filifactor alocis (35896) | Streptococcus gordonii (10558) |
| Fusobacterium necrophorum (25286) | Streptococcus intermedius (27335) |
| Fusobacterium nucleatum ss nucleatum (25586) | Streptococcus mitis (49456) |
| Fusobacterium nucleatum ss polymorphum (10953) | Streptococcus mutans (25175) |
| Fusobacterium nucleatum ss vincentii (49256) | Streptococcus oralis (35037) |
| Fusobacterium periodonticum (33693) | Streptococcus parasanguinis (15912) |
| Fusobacterium naviforme (25832) | Streptococcus pneumoniae (49619) |
| Gemella haemolysans (10379) | Streptococcus salivarius (27945) |
| Gemella morbillorum (27824) | Streptococcus sanguinis (10556) |
| Granulicatella adjacens (49175) | Streptococcus vestibularis (49124) |
| Haemophilus aphrophilus (33389) | Tannerella forsythia (43037) |
| Haemophilus influenza (33533) | Veillonella dispar (17748) |
| Haemophilus paraaphrophilus (29242) | Veillonella parvula (10790) |
| Haemophilus segnis (33393) | Treponema denticola (B1) |
| Lactobacillus acidophilus (4356) | Veilonella atypica (17744) |
| Lactobacillus brevis (14869) | Bifidobacterium denticum (27534) |
| Lactobacillus fermentum (14931) | Moraxella catarrhalis (24240) |

All cultivated strains were obtained from the American Type Culture Collection (ATCC number in parenthesis), except for Treponema denticola (B1), which was obtained from The Forsyth Institute.
* ATCC strains 43718 and 29523,
** ATCC strains 11827 and 11828.

Relation of complementary sequence concentration to bacterial counts was determined. The levels of bacterial taxa in each sample were determined by comparison with standards containing known picomolar levels of sequences complementary to the detection probe sequences (e.g., between about 0.004 and about 0.04 pM). The equivalency between cell numbers and picomolar levels of complementary sequences was individually assessed for 20 cultivated bacterial species (Table 2). These samples were laid on a nylon membrane and probed using the corresponding specific oligonucleotide probes. It was observed that, on average, 10⁶ bacterial cells were equivalent to 0.068±0.048 μM (mean±SD).

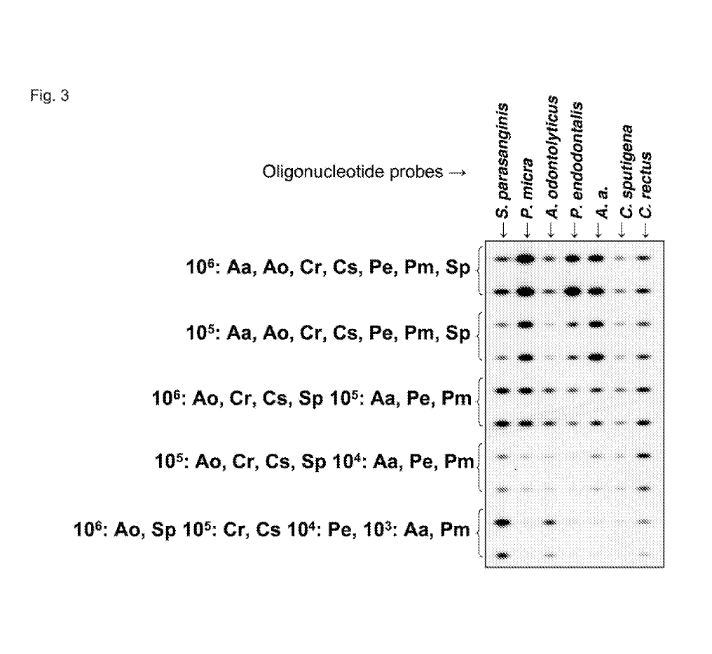

The effect of multiple species at different levels on signal detection was studied. The presence of one or more species in a sample at high levels might affect the detection of other taxa present at lower levels. Since there is marked variation in species concentrations in single samples of subgingival biofilms, the presence of high numbers of cells from a given species were studied to determine if it would overshadow the presence of less abundant taxa. To evaluate this possibility, mixtures of bacterial species at different levels were prepared. FIG. 3 demonstrates that TNAs from individual bacterial species could be detected in the range of 10⁴-10⁶ cells when in the presence of different levels of cells of other species. For instance, even in the presence of 10⁶ cells of Actinomyces odontolyticus and Streptococcus parasanguinis, 10⁴ cells of Porphyromonas endodontalis and 10³ cells of Aggregatibacter actinomycetemcomitans and Parvimonas micra could be detected.

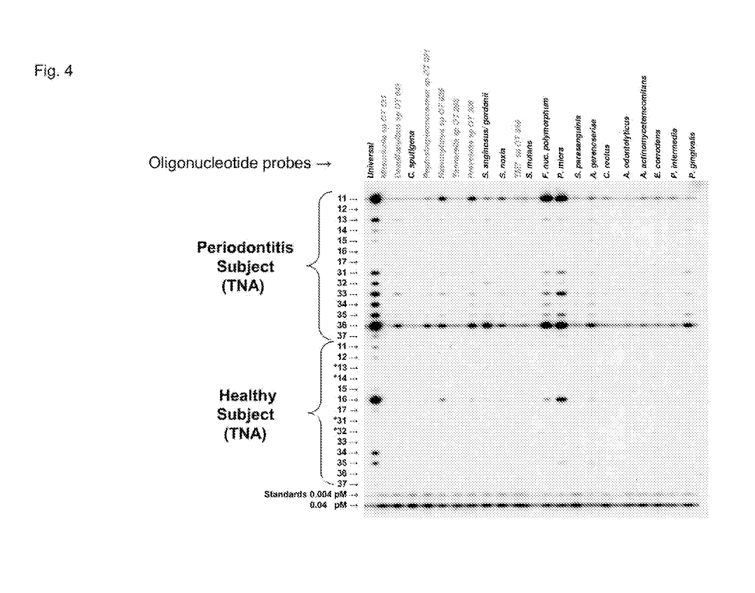

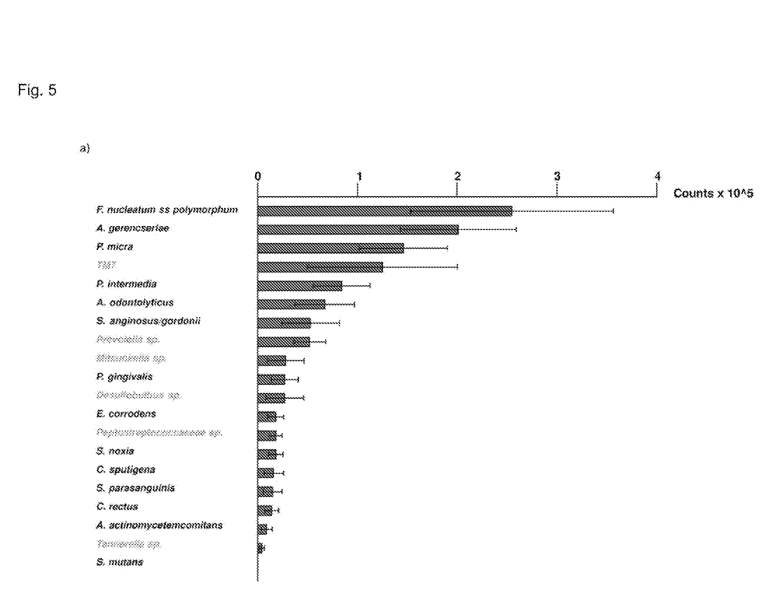

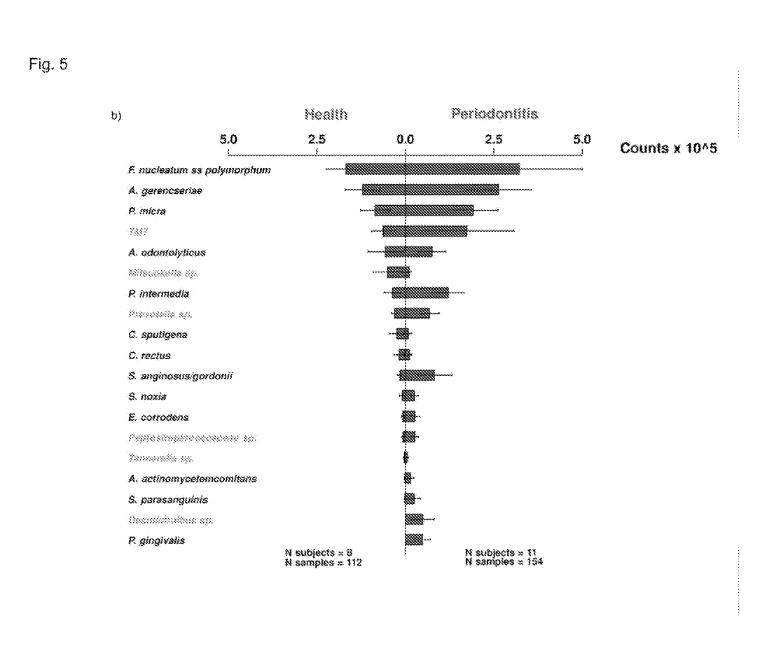

Enumeration of taxa in clinical samples is encompassed by the methods of the present invention. To assess the feasibility of the method, cultivated and uncultivated bacterial taxa could be detected and quantified. FIG. 4 shows an example of a typical membrane from this study described herein. The use of a universal (eubacterial) probe gave an estimate of the total bacterial load in each sample. Samples from periodontitis patients usually showed higher levels of subgingival bacteria based on the universal probe signals than samples from periodontally healthy subjects. Overall, the most commonly detected uncultivated/unrecognized species in the subject population were TM7 sp OT 346, Prevotella sp OT 306, Mitsuokella sp OT 131 (FIG. 5a). In the cultivated segment of the microbiota, the most abundant taxa were Fusobacterium nucleatum ss polymorphum, Actinomyces gerencseriae and P. micra. When the data from periodontal health and disease were compared, different mean microbial profiles were observed (FIG. 5b). Periodontitis patients showed higher mean counts of Prevotella sp OT 306. Desulfobulbus sp OT 041 was detected only in the periodontitis group. Mean counts of Prevotella intermedia, a member of the “orange complex also were elevated in this group. Porphyromonas gingivalis, a periodontal pathogen of the “red complex” could only be detected in the periodontitis group.

It has long been recognized that many taxa in subgingival plaque were not being cultivated, as there were marked differences between total viable counts (representing cultivated species) and total microscopic counts representing all organisms. This phenomenon has been described as the “great plate count anomaly” and it seems to be a constant in bacterial samples originating from different environments, including the oral cavity.

Periodontal pathogens are among the uncultivated segment of the microbiota. Certain studies have used cloning to seek novel and uncultivated bacterial species. Cloning, however, is time-consuming, limiting the number of samples and clones that can be analyzed at a time and it does not allow direct quantification. Recently, next generation sequencing (NGS) methods have surpassed the capabilities of cloning and enabled deeper coverage and less labor intensive sequencing of microbial communities. Even though NSG represents a major advance in the study of oral microbiology, the method involves steps that are known to introduce biases in the resulting microbial profile, including using aliquots of a sample, the amplification of samples and, often times, the pooling of samples. Furthermore, those studies report data at the genus level, preventing the identification of pathogenic or health compatible species.

Since the differences between periodontal health and disease, and before and after therapy are quantitative and due to the site-specific nature of periodontal diseases, quantification of individual biofilm samples is needed. Species quantification in samples from periodontal sites with different clinical status in the same or different oral cavities is a powerful first step in discriminating pathogens from host-compatible species. High throughput is another prerequisite. Species counts are highly variable in biofilm samples, requiring the analysis of large numbers of samples from many subjects in order to detect meaningful differences in species counts. In the present study, the standards for quantification of individual taxa contained 0.004 and 0.04 μM of the sequences complementary to the probes. These levels were estimated based on the molecular weight of the 16S rRNA molecule and the number of copies thought to be present in bacterial cells. Subsequent experiments indicated that 0.04 μM yielded signals equivalent to about 0.44×10⁵ bacterial cells. rRNA was selected as the target molecule because it is more abundant in bacterial cells than DNA, and thus could enhance the sensitivity of oligonucleotide probes. Additionally, because an actively growing cell has 10³-10⁴ rRNA molecules, rRNA is more associated with cell viability than DNA. Hence, the use of rRNA can provide insights on the relevance of the test species in the ecosystem of interest and avoid biases in the results due to the presence of dead cells.

Levels of periodontal bacteria in single curette stroke samples of subgingival plaque commonly range from 10⁴ to 10⁷ cells. ROQT is able to consistently detect 10⁵ bacterial cells, and 10⁴ cells of most species (FIG. 1). This is in line with the level of sensitivity obtained using whole genomic DNA probes.

Uncultivated species of the subgingival microbiota of periodontal health and disease have been evaluated. The most abundant species belonged to the genera Selenomonas, Streptococcus, Veillonella, Campylobacter and Peptostreptococcus. Fusobacterium species and Actinomyces species, both routinely found by culture and DNA probes were rarely detected. In contrast, in our study, F. nuc. ss polymorphum and A. gerencseriae were the most abundant taxa detected. A robust association of peptostreptococci with periodontitis: Peptostreptococcus strains BS044 (not in HOMD) and CK035 (HOMD: Peptostreptococcus stomatis) were found to be very numerous. Targeted DNA approaches have found Parvimonas micra (formerly Peptostreptococcus micros) to be elevated in periodontitis patients. In the data presented herein, P. micra was also more prevalent in periodontitis samples. Desulfobulbus sp CH031 (not in HOMD) and OT 041 have been significantly associated with deep periodontal sites. In the work described herein, although in low numbers, Desulfobulbus sp OT 041 was only detected in periodontitis samples. Selenomonas clones were not associated with periodontal disease in another study. In the data described herein, S. noxia was present in low numbers in healthy subjects and Selenomonas sp CS002 (HOMD: Mitsuokella sp OT 131) was increased in samples from healthy individuals. P. gingivalis, Treponema denticola and Tannerella forsythia were rarely detected in periodontitis samples by cloning, which contrasts with investigations using culture, PCR, real time PCR, immunofluorescence, in situ hybridization, immunocytochemistry, DNA probes and oligonucleotide probes. In the data presented herein, P. gingivalis was solely detected in periodontitis patients and accounted for a significant portion of the total probe count (FIG. 5b).

In the data described herein, Tannerella sp OT 286 was the least abundant uncultivated taxon present in periodontitis patients, corroborating findings by others. TM7 sp OT346 was the most prevalent uncultivated phylotype overall and in the periodontitis group, which is in accord with previous reports. The Human Oral Microbe Identification Microrray (HOMIM) has been employed to study the subgingival microbiota of periodontal health and disease. The frequency of detection of P. gingivalis, S. noxia, S. anginosus/gordonii, A. gerencseriae and TM7 OT 346 was significantly higher in diseased sites and the frequency of Capnocytophaga sputigena was elevated in healthy sites.

The methods described herein include methods for detecting and quantifying uncultivated bacterial species in subgingival biofilm samples in periodontal health and disease. The specificity of the probes used was confirmed by the absence of cross-reactions with any of the 96 bacterial taxa tested, representing the most prominent cultivable oral bacterial taxa. The small clinical study demonstrated the feasibility of the method for its use in clinical trials. The strengths of the proposed method include the absence of pooling, amplification or dilution bias, since an entire individual sample is laid onto the membrane. It allows the quantification of both cultivable and uncultivable bacterial taxa. It is high throughput, in that multiple samples can be analyzed for the levels of multiple species at the same time on a single membrane and it is relatively inexpensive. The method also has certain considerations. The standard curve presented has 3 data points, which enables quantification of taxa in the 10⁴-10⁷ cells range in a given sample. However, additional levels of standards can be added to provide a tighter or more comprehensive standard curve. The data presented above was obtained using film exposure, which has a dynamic range of about 2 orders of magnitude. Encompassed in the methods described herein is the switch to image capture by a CCD camera, reaching a dynamic range of 4.8 orders of magnitude and enhancing the accuracy of the method. Although the format of the ROQT resembles that of the checkerboard DNA-DNA hybridization technique, it is not meant to represent a “more sensitive checkerboard”. The techniques differ in the nature of the probes, their target molecules, their hybridization protocols and the nature of the species sought.

It seems that an ever growing number of taxa can be detected in the oral cavity and more than 35% of them cannot be cultivated. It is likely that only a subset of this segment is relevant to disease development. ROQT provides the ability to examine large numbers of biofilm samples from large numbers of subjects for the levels of uncultivated taxa. This approach can indicate the most numerous, and thereby possibly the most relevant, taxa associated with periodontal diseases, clarifying their potential role in initiation and progression of periodontal infections. By identifying the more relevant uncultivated/unrecognized taxa, this technique will guide the isolation and cultivation of disease-associated uncultivated and unrecognized taxa. Such taxa merit further pursuit by cultivation, in order to evaluate the pathogenic mechanisms of the selected taxa and to develop more targeted treatment and prevention strategies.

The present invention relates to methods for quantifying the amount of a (e.g., one or more) cultivated and/or uncultivated/unidentified microorganism. The present invention pertains to extracting the total nucleic acid from a sample, allowing the TNA to hybridize to probes and quantifying the amount of TNA in the sample. The quantification of bacteria, whether cultivated or uncultivated, allows for more accurate determination of the presence or absence of a disease state, a healthy state or both.

Probes used in the study can be determined using techniques known in the art. The nucleic acid sequence of these molecules can be determined by studying the divergent regions of the genome of these microorganisms, in particular the 16S rRNA genes, and testing them for their ability to identify the target microorganism. Using these 16S sequences, nucleic acid molecules (e.g., probes) were designed and prepared. for use in the identification of microorganisms. Specifically, using the protocol described in U.S. patent application Ser. No. 11/556,296, about 94 oligonucleotide sequences were designed, prepared and labeled, and these molecules are used to identify the microorganisms, as shown in FIG. 6A-6R. In particular, the technique identifies bacteria typically found in the oral cavity. Specifically, the probes described herein detect one or more microorganisms by detecting nucleic acid molecules in the sample, either bacterial 16S rRNA or 16S rRNA genes. Arrays for microflora common to other areas (e.g., lungs, blood, skin, vagina, urinary tract, intestinal tract) of the human body are also embodied by the present invention.

The present invention includes methods quantifying the flora composition of microorganisms in a sample. Specifically, the method includes contacting nucleic acid molecules obtained from a sample with the probes of the present invention. This step occurs under conditions suitable for hybridization to form a complex or hybrid, and the hybrids are detected. The presence of complexes correlate with the microorganisms listed in Table of FIG. 6, to thereby provide a composition of the microflora of the sample. Intensity of the hybridization signals (representing hybrids) in samples is compared with that of the standards for quantification. Those standards contain known amounts of sequences that are complementary to the probe sequences and correspond to levels of TNA present in known amounts of bacterial cells.

Such an analysis is helpful in identifying novel microbial pathogens, specifically those that have never been identified or cultivated. Such identification can guide the characterization of new bacterial species and will have an impact in strategies for prevention, diagnosis and treatment of diseases. This analysis can also be useful to assess the disease state and/or healthy state of tissue, to monitor the effects of treatment as well as to investigate the impact of systemic conditions, such as obesity and diabetes, on other diseases such as periodontal diseases. For example, one could test the effect of a mouthwash (or a toothpaste, fluoride, breath enhancers, tooth-whitening treatments or floss) on an individual by obtaining samples before and after using the mouthwash and comparing the amount of specific flora present in each sample (e.g., the number and/or type of bacterial present or absent in the sample). Comparing the amounts of specific bacteria present in compositional flora of each sample allows one to make determinations as to the efficacy of the product. As such, the present invention includes assessing the effect of an oral product on the compositional flora of a sample at one or more time points, and assessing or comparing the amounts of one or more microorganisms, as described herein.

The methods of the present invention further embody assessing the efficacy of an oral product independent of the specific microorganism or groups of microorganisms identified. In this embodiment, the amount of TNA present in the sample correlates directly to oral health, or to a particular disease or condition, as described further herein. Such a method involves hybridizing the TNA in a sample to one or more probes, and determining the levels the one or more complexes at the one or more time points (e.g., before and after administration of the oral product). Absence is defined herein as the level of a hybrid complex that is below a detectable level or limit. Based on the amount of cultivated and/or uncultivated bacteria in the sample, a determination of the efficacy of the oral product can be made.

Similarly, the methods of the present invention relate to methods of diagnosing patients with a disease or condition, providing a prognosis for a patient, and/or determining the efficacy of treatment. In an embodiment, methods of diagnosing a patient with a disease or condition can be conducted by determining the presence or absence of the microorganism associated with the disease or condition, as described herein. Once the microbe(s) of a particular sample is quantified, an individual can be better diagnosed and/or treated for diseases associated with those microbes. Amounts of TNA present in levels greater than 10³ indicate the presences or absence of a disease state or healthy state. In a particular embodiment, the amounts of bacteria, cultivated or uncultivated, present in the range between 10⁴ to about 10⁶ indicate a disease state or a healthy state. In yet another embodiment, the amount of bacteria present ranges between about 0.004 and about 0.5 picomolars (pM) (e.g., 0.04, 0.16 and 0.32). For example, a diseased sample that contains the following bacteria in a range between 10⁵ to about 10⁶ indicates, in certain embodiments, that the individual has periodontal disease: Fusobacterium nucleatum. ss polymorphum, Actinomyces gerencseriae, Mitsuokella sp OT 131, Prevotella sp OT 306, Porphyromonas gingivalis and Desulfobulbus sp OT 041. The results of such a test help a dentist or doctor properly diagnose the disease, and can impact the type of treatment provided to the patient. In yet another embodiment, quantity of TNA of bacteria in the sample can directly correlate with the presence of a disease or condition (e.g., a diagnosis). Such methods include determining the quantity of TNA of one or more bacteria in a sample, and then determining diseases associated with that pattern (presence and/or absence) of nucleic acid molecules in the sample.

Furthermore, the methods of the present invention include monitoring treatment of diseases. For example, the treatment for a periodontal patient above can be monitored after the patient has received the proper treatment with antibiotics, surgery, and/or other dental treatment. As such, one can compare the results of a baseline determination, with one or more determinations made after treatment has begun. In one example, the amount or level (e.g., the level goes from one level to a lower level or even an undetectable level) of certain nucleic acid sequences from the sample indicates that treatment is working. Increases in certain levels of TNA from bacteria associated with the disease or condition in a sample invention indicate, in an embodiment, that treatment is not effective. Assessing levels at various stages or time points prior to and/or during the course of treatment provides a physician/dentist with information to make better, more informed decisions regarding treatment. Assaying the nucleic acid molecules of the present invention can be conducted using several methods. Briefly, blot or hybridization techniques include immobilizing or attaching nucleic acid molecules to a solid support (e.g., a nylon membrane), and subjecting or contacting nucleic acid molecules obtained from a sample under conditions for hybridization. Methods for preparing the nucleic acid molecules from the sample are further described herein. In nucleic acid hybridization reactions, the conditions used to achieve a particular level of stringency are described herein and depend on the nature of the nucleic acids being hybridized. For example, the length (e.g., 18-24 mer), degree of complementarity, nucleotide sequence composition (e.g., GC v. AT content), and nucleic acid type (e.g., RNA v. DNA v. PNA) of the hybridizing regions of the nucleic acids can be considered in selecting hybridization conditions.

In an embodiment, the methods described herein do not need amplification because the total amount of nucleic acid in the sample is being assessed, and not an amount being amplified. Also, samples are not diluted, rather placed entirely on a solid support.

In a preferred embodiment, methods for identifying a nucleic acid sequence involve the use of an array. An “array,” “microarray,” “DNA chip,” “biochip,” or “oligo chip” may be used interchangeably and refers to a grid of spots or droplets of genetic material of known sequences in defined locations or known positions. The advantage of using an array is the ability to test a sample against hundreds of nucleic acid sequences at once. The array of probes can be laid down in rows and columns. For example, five arrays (64×64 droplets) are arranged on a nylon membrane, and the same array is repeated three times. The actual physical arrangement of probes on the chip is not essential, provided that the spatial location of each probe in an array is known. When the spatial location of each probe is known, the data from the probes can be collected and processed. In processing the data, the hybridization signals from the respective probes can be reasserted into any conceptual array desired for subsequent data reduction whatever the physical arrangement of probes on the chip.

The genetic material is systematically arranged on a solid support that includes, e.g., glass, silica chips, nylon (polyamide) membrane, polymer, plastic, ceramic, metal, coated on optical fibers, or infused into gel, matrix. Examples of the type of solid support can be a slide, plate, chip, dipstick, or other types known in the art or later developed. The solid support can also be coated to facilitate attachment of the genetic material (such as TNA) to the surface of the solid support. Any of a variety of methods known in the art may be used to immobilize nucleic acids to a solid support. They can be attached directly to the solid supports by epoxide/amine coupling chemistry. Another commonly used method consists of the non-covalent coating of the solid support with avidin or streptavidin and the immobilization of biotinylated oligonucleotide probes. By oligonucleotide probes is meant nucleic acid sequences complementary to a species-specific target sequence.

Using a solid support having the nucleic acid molecules from a sample bound thereto, the method of the present invention involves contacting the nucleic acid molecules with oligonucleotide probes to be tested under conditions suitable for hybridization with one another. A sample is obtained from the individual to be tested and can consist of saliva, plaque, swab, sputum, aspirate, blood, plasma, cerebrospinal fluid, aspirate, tissue, skin, urine, mucus, or cultured organisms grown in vitro. The sample obtained can be related to the type of array that is being utilized. For example, in the case of an array for the oral cavity, a plaque sample is preferable and can be obtained by scraping the plaque with a sterile instrument.

In certain embodiments, the nucleic acid (e.g., TNA) can be obtained from a sample and labeled by, e.g., universally labeled probes that hybridize to a portion of the TNA. The labeled TNA is subjected to or contacted with the nucleic acid molecules of the present invention under conditions suitable for hybridization, as further described herein. Hence, a complex forms between the TNA from the sample, the labeled universal probe and the probes. The complex is then detected as described herein.

Alternatively, a label can be added directly to the original TNA in the sample. Means of attaching labels to nucleic acids include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. The most frequently used labels are digoxigenin (and appropriate enzymes and substrates), fluorochromes like Cy3, Cy5 and Cy7 suitable for analyzing an array by using commercially available array scanners (e.g., Axon, General Scanning, and Genetic Microsystem). Other labels that can be used in the present invention include biotin for staining with labeled streptavidin conjugate, magnetic beads (e.g., Dynabeads™), dendrimers, fluorescent proteins and dyes (e.g., fluorescein, texas red, rhodamine, green fluorescent protein, and the like, see, e.g., Molecular Probes, Eugene, Oreg., USA), radioactive labels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include WO 97/27317, and U.S. Pat. Nos. 3,817,837; 3,850,752; 3,939,350; 3,996,345; 4,277,437; 4,275,149; and 4,366,241.

Fluorescent and chemiluminescent detection can be employed, using the appropriate scanners or CCD cameras. The oligonucleotide probes can all be labeled with a single label, e.g., a single fluorescent or chemiluminescent label. Alternatively, in another embodiment, different oligonucleotide probes can be simultaneously hybridized where each probe has a different label. For instance, one set of probes could have a green fluorescent label and a second set of probes could have a red fluorescent label. The scanning step will distinguish sites of binding of the red label from those binding the green fluorescent label. Each probe set can be analyzed independently from one another.

Once the sample is prepared, it can be subjected to the nucleic acid molecules of the present invention for hybridization. Hybridization refers to the association of single strands of polynucleotides through their specific base-pairing properties to form a complementary double-stranded molecule. With respect to the present invention, the labeled TNA of the sample hybridizes with the oligonucleotides on the solid support. Hybridization conditions include variables such as temperature, time, humidity, buffers and reagents added, salt concentration and washing reagents. Preferably, hybridization occurs at high stringency conditions (e.g., 55° C., for 16 hours, 3×SSC). Examples of stringency conditions are described herein. Methods for hybridization are known, and such methods are provided in U.S. Pat. No. 5,837,490, by Jacobs et al. The solid support can then be washed one or more times with buffers to remove unhybridized nucleic acid molecules. Hybridization forms a complex between the nucleic acid of the present invention and nucleic acid of the sample.

Hybridization assay procedures and conditions will vary depending on the application and are selected in accordance with the general binding methods known including those referred to in: Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623.

The complex or hybrid, which is labeled, can be detected and quantified using the standards for quantification included in each assay. Detection of the array can be performed by autoradiography or by a chemiluminesce/fluorescence imaging system in real time to determine the presence of hybridized products at particular locations on the solid support. In particular, detection can occur using scanners that use CCD cameras (such as the GBOX IChemi) or scanners that emit light from a laser at specific frequency (such as the Typhoon imager or the Axon Gene Pix). Scanners and other devices, including those known and later developed, for detecting the labeled hybridized complexes can be used. These measurements are converted to electronic signals that can be analyzed. The raw data optionally are filtered and/or normalized. Filtering refers to removing data from the analysis that does not contribute information to the experimental outcome, e.g., does not contribute to the identification of a microbe. Normalizing data refers to, in one embodiment, a linear transformation to correct for variables within the experimental process.

The data can be analyzed by a qualified person or computerized system. In an embodiment, the presence of hybridization of the nucleic acid molecules of the present invention correlates to the presence of the corresponding microbe in the sample. One can compare the spot having a detectable hybrid complex, against a table or database containing information about the spots on which the nucleic acid molecules were bound, and with which particular microorganism they correlate. FIG. 6 has a table, for example, that lists the microorganisms and the sequence of the probe to which they correlate. After such a comparison, the microorganism can be identified and quantified in the sample. One or more nucleic acid molecules can correlate to a particular microorganism, closely related microorganisms, or genus. In some embodiments, at least 2 probes correlate to or identify a microorganism, as defined herein. Having more than one occurrence of hybridization with more than one probe can, in some embodiments, provide for a more accurate identification.

The presence of hybridization, as detected in some embodiments by chemiluminescence or fluorescence, is compared to controls. In the work described herein, a universal probe was included in the assay as a positive control as well as a means to determine the bacterial load of each sample. The universal probe identifies a section of 16S rRNA that is common in all microorganisms that are being tested. Such a control not only shows that the hybridization is occurring, but it is occurring in various areas throughout the array. Negative controls can also be used. In the work described herein, areas of the assay in which probes or samples were not included (“blank” lanes) were used as negative controls. Such controls assist in determining the existence of any background noise (e.g., fluorescence, chemiluminescence). Negative controls, in an embodiment, can also include a 16S rRNA probe for an organism only found in non-human environments such as acid mine drainage, or hyperthermal ponds. The technology, as described herein, allows for the identification and quantification of a number (e.g., at least about 10, 20, 30, 50, 100, 150, 200 and the like) of microorganisms at one time. In a particular embodiment, about 22 microorganisms were identified and/or quantified.

The terms “microorganism” and “microbe” are used in its broadest sense and include those that are known and named, and those that have not yet been named or cultivated. The term “microorganism” includes single species, phylotypes, closely related species or phylotypes, genus, and higher taxon. As a general rule, bacterial strains of species or phylotypes have less than about a 2% difference in 16S rRNA. Closely related species or phylotypes generally have between about a 2% and about a 4% difference in 16S rRNA, whereas a genus often has between about a 5% and about a 10% difference in 16S rRNA. These are simply general guidelines. The probes identify specific species/phylotypes of microorganisms, closely related species and in some cases a particular genus. As used herein, the phrase “identifying a microorganism” refers to the determination of the genus, closely related microorganisms, as well as the species/phylotype of a microorganism, including those that are known, unnamed or uncultivated (e.g., those known from strains or 16S rRNA clones).

Examples of microorganisms are found in FIG. 6, and include Gram negative aerobic bacteria, Gram positive aerobic bacteria, Gram negative microaerophilic bacteria, Gram positive microaerophilic bacteria, Gram negative facultatively anaerobic bacteria, Gram positive facultatively anaerobic bacteria, Gram negative anaerobic bacteria, Gram positive anaerobic bacteria, Gram positive asporogenic bacteria, Actinomycetes. Uncultivated or unnamed microorganisms can also be identified by the methods described herein. Uncultivated microorganisms are described by its similarity of the nucleic acid molecules used in the assay of the present invention to the sequence of the microorganism's 16S rDNA in a public sequence database, such as GenBank. Additionally, “microorganism” refers to live, dead, fragmented or lysed organisms.

As used herein, the terms “nucleic acid molecule” include both sense and anti-sense strands, cDNA, complementary DNA, recombinant DNA, RNA, wholly or partially synthesized nucleic acid molecules, PNA and other synthetic DNA homologs. Total Nucleic Acid (TNA) refers to all of nucleic acid molecules in the sample. A nucleotide “variant” is a sequence that differs from the recited nucleotide sequence in having one or more nucleotide deletions, substitutions or additions so long as the molecules binds to the nucleic acid molecules of the present invention including its reverse complement. Such variant nucleotide sequences will generally hybridize to the recited nucleotide sequence under stringent conditions.

As used herein, an “isolated” gene or nucleotide sequence which is not flanked by nucleotide sequences which normally (e.g., in nature) flank the gene or nucleotide sequence (e.g., as in genomic sequences). Thus, an isolated gene or nucleotide sequence can include a nucleotide sequence which is designed, synthesized chemically or by recombinant means.

Also encompassed by the present invention are nucleic acid sequences, DNA or RNA, PNA or other DNA analogues, which are substantially complementary to the DNA sequences and which specifically hybridize with their DNA sequences under conditions of stringency known to those of skill in the art. As defined herein, substantially complementary means that the nucleic acid need not reflect the exact sequence of the sequences of the present invention, but must be sufficiently similar in sequence to permit hybridization with nucleic acid sequence of the present invention under high stringency conditions. For example, non-complementary bases can be interspersed in a nucleotide sequence, or the sequences can be longer or shorter than the nucleic acid sequence of the present invention, provided that the sequence has a sufficient number of bases complementary to the DNA of the microorganism to be identified to allow hybridization therewith.

Specific hybridization can be detected under high stringency conditions. “Stringency conditions” for hybridization is a term of art which refers to the conditions of temperature and buffer concentration which permit and maintain hybridization of a particular nucleic acid to a second nucleic acid; the first nucleic acid may be perfectly complementary to the second, or the first and second may share some degree of complementarity which is less than perfect. For example, certain high stringency conditions can be used which distinguish perfectly complementary nucleic acids from those of less complementarity. “High stringency conditions” for nucleic acid hybridizations and subsequent washes are explained, e.g., on pages 2.10.1-2.10.16 and pages 6.3.1-6 in Current Protocols in Molecular Biology (Ausubel, et al., In: Current Protocols in Molecular Biology, John Wiley & Sons, (1998)). The exact conditions which determine the stringency of hybridization depend not only on ionic strength, temperature and the concentration of destabilizing agents such as formamide, but also on factors such as the length of the nucleic acid sequence, base composition, percent mismatch between hybridizing sequences and the frequency of occurrence of subsets of that sequence within other non-identical sequences. Thus, high stringency conditions can be determined empirically.

By varying hybridization conditions from a level of stringency at which no hybridization occurs to a level at which hybridization is first observed, conditions which will allow a given sequence to hybridize (e.g., selectively) with the most similar sequences in the sample can be determined. Exemplary conditions are described in the art (Krause, M. H., et al., 1991, Methods Enzymol. 200:546-556). Also, low and moderate stringency conditions for washes are described (Ausubel, et al., In: Current Protocols in Molecular Biology, John Wiley & Sons, (1998)). Washing is the step in which conditions are usually set so as to determine a minimum level of complementarity of the hybrids. Generally, starting from the lowest temperature at which only homologous hybridization occurs, each C by which the final wash temperature is reduced (holding SSC concentration constant) allows an increase by 1% in the maximum extent of mismatching among the sequences that hybridize. Generally, doubling the concentration of SSC results in an increase in Tm of about 17 C. Using these guidelines, the washing temperature can be determined empirically for high stringency, depending on the level of the mismatch sought. In some embodiments, high stringency conditions include those in which nucleic acid with less than a few mismatches does not bind. Specific high stringency conditions used to carrying out the steps of the present invention are described in the Exemplification. High stringency conditions, using these guidelines, lie in a temperature range between about 40° C. and about 60° C., an SSC concentration range between about 1× and about 10× (e.g., about 2×), and a reaction time range of between about 30 seconds and about 36 hours.

### EXEMPLIFICATION

### Example 1

### Objective

### To Develop a High-Throughput Method to Quantify Uncultivated/Unrecognized Microorganisms in Biofilm Samples Material and Methods

### Sample Preparation

Two types of samples were employed: a) total nucleic acids extracted from bacterial cells and b) total nucleic acids extracted from subgingival biofilm samples.

a) Bacterial Cells

Due to the unavailability of cells from uncultivated/unrecognized bacterial species, cells from cultivated taxa were used as test species for the development of the method and validation purposes in this study.

The majority of strains (Table 1) were grown on Trypticase soy agar supplemented with 5% defibrinated sheep blood (Baltimore Biological Laboratories (BBL), Cockeysville, Md.). Tannerella forsythia was grown on Trypticase soy agar supplemented with 5% sheep blood and 10 μg/ml N-acetylmuramic acid (Sigma Chemical Co., St. Louis, Mo.). Porphyromonas gingivalis was grown on Trypticase soy agar supplemented with 5% sheep blood, 0.3 μg/ml menadione (Sigma) and 5 μg/ml hemin (Sigma). Eubacterium and Neisseria species were grown on Fastidious Anaerobic Agar (BBL) with 5% defibrinated sheep blood. Treponema denticola and Treponema socranskii were grown in Mycoplasma broth (Difco Laboratories, Detroit, Mich.) supplemented with 1 mg/ml glucose, 400 μg/ml niacinamide, 150 μg/ml spermine tetrahydrochloride, 20 μg/ml Na isobutyrate, 1 mg/ml L-cysteine, 5 μg/ml thiamine pyrophosphate and 0.5% bovine serum. All strains were grown at 35° C. under anaerobic conditions (80% N₂, 10% CO₂, 10% H₂).

Bacterial cells were harvested from agar plates, placed into 100 μA of RNAse-free TE buffer (10 mM Tris-HCl, 0.1 mM EDTA, pH 7.6) and kept at −80° C. until extraction of total nucleic acids (TNA).

b) Subgingival Plaque Samples

Subgingival plaque samples were collected from healthy subjects and periodontitis patients. Samples were taken from mesio-buccal sites using 11/12 sterile Gracey curettes (HuFriedy, Chicago, Ill.) and placed in individual microcentrifuge tubes containing 100 μl of RNAse-free TE buffer. Samples were kept at −80° C. until TNA extraction.

Extraction of Total Nucleic Acids

TNA extraction from all samples was performed using a Masterpure RNA purification kit (Epicentre, Madison, Wis.). Cells were pelleted by centrifugation at 3,500 rpm for 10 min. After the supernatant was discarded, the pellet contained in 25 μl of TE was re-suspended by vortex mixing. One microliter of proteinase K (50 μg/μl) and 300 μl of tissue and cell lysis buffer (provided by the manufacturer) were added and the solution was incubated in a 65° C. waterbath for 15 min. After 5 min in ice, 175 μl of MCP protein precipitation reagent (provided by the manufacturer) were added to each sample. The debris were pelleted by centrifugation at 12,000 rpm for 10 min. The supernatant was transferred to a new tube and 500 μl of isopropanol were added. Tubes were mixed by inversion for 2 min and TNAs were pelleted by centrifugation at 12,000 rpm for 10 min at 4° C. After carefully pouring off the isopropanol, pellets were rinsed twice with 70% ethanol. Pellets were air dried for 10 min and re-suspended in 35 μl of TE buffer at 37° C. for 10 min. 1 μl of ScriptGuard (Epicentre) was added to each sample. Samples were kept at −80° C. until analysis. TNAs from individual bacterial species were measured with a spectrophotometer (Nanodrop, Wilmington, Del.) at 260 nm wavelength. TNAs from bacterial mixtures and from clinical samples were not measured, rather, the entire sample was laid onto a positively charged nylon membrane.

90 μl of 2% glutaraldehyde and 910 μl of 6×SSC (1×SSC=150 mM NaCl, 15 mM Na citrate, pH 7.0) were added to each sample. The final solutions were deposited in individual lanes of a Minislot (Immunetics, Cambridge, Mass.), concentrated onto a nylon membrane (Boehringer Mannheim) by vacuum and fixed onto the membrane by crosslinking using ultraviolet light (Stratalinker 1800, La Jolla, Calif.) and dried at room temperature. The Minislot device permitted the deposition of 28 different plaque samples in individual “lanes” on a single 15×15 cm nylon membrane as well as 2 control lanes containing standards for quantification of each test species.

Probe Preparation

Oligonucleotide probes were prepared for 23 cultivated and 19 uncultivated bacterial taxa. The sequences were 18 to 22 nucleotides in length and had minimal secondary structure. Each sequence included in this group of probes targeted the 16S rDNA gene of the species or phylotypes listed in Table 2. The probe panel also included one universal (eubacterial) probe. This sequence was based on a conserved region of the bacterial 16S rDNA gene. All probes used in this study were based on sequences routinely employed in the Human Oral Microbial Identification Microarray (HOMIM). The full list of probe sequences has been published elsewhere (Colombo, A. P., et al. J Periodontol 80: 1421-1432 (2009)) 100 pM of each sequence were labeled using a Digoxigenin 3′ end labeling kit (Roche, Indianapolis, Ind.).

TABLE 2
Taxa for which oligonucleotide probes have been validated.

| Cultivated | Uncultivated/Unrecognized |
|---|---|
| Aggregatibacter actinomycetemcomitans *^{a} | Acidaminococcus [G-1] oral taxon 135 |
| Actinomyces gerencseriae *^{a} | Acidaminococcus [G-1] oral taxon 148 |
| Actinomyces odontolyticus *^{a} | Bacteroidetes [G-2] sp. oral taxon 274 |
| Campylobacter concisus ^{a} | Capnocytophaga sp. oral taxon 326 |
| Campylobacter rectus *^{a} | Capnocytophaga sp oral taxon 329 |
| Capnocytophaga sputigena *^{a} | Capnocytophaga sp. oral taxon 336 |
| Eikenella corrodens *^{a} | Desulfobulbus sp. oral taxon 041 * |
| Eubacterium brachy ^{a} | Mitsuokella sp oral taxon 131 * |
| Fusobacterium nucleatum ss. polymorphum *^{a} | Neisseria sp. oral taxon 020 |
| Gemella haemolysans ^{a} | Peptostreptococcaceae [11][G-7] sp. oral taxon 081 * |
| Haemophilus parainfluenza | Peptostreptococcaceae [13][G-1] sp. oral taxon 113 |
| Parvimonas micra *^{a} | Porphyromonas sp. oral taxon 279 |
| Porphyromonas endodontalis ^{a} | Prevotella sp. oral taxon 292 |
| Porphyromonas gingivalis *^{a} | Prevotella sp. oral taxon 306 * |
| Prevotella denticola ^{a} | Streptococcus sp. oral taxon 055 |
| Prevotella intermedia *^{a} | Streptococcus sp. oral taxon 057 |
| Selenomonas noxia *^{a} | Streptococcus sp. oral taxon 066 |
| Shuttleworthia satelles | Tannerella sp. oral taxon 286 * |
| Streptococcus anginosus * | TM7 [G-1] sp. oral taxon 346 * |
| Streptococcus gordonii *^{a} | |
| Streptococcus mutans *^{a} | |
| Streptococcus parasanguinis *^{a} | |
| Tannerella forsythia ^{a} | |
| Treponema denticola | |

A subset of the probes employed were “combination probes”, in that they could not distinguish species/phylotypes. This was the case for S. anginosus/gordonii; C. rectus/concisus and Streptococcus sp OT055/057/066.
(*): Indicates taxa for which probes were used in the pilot clinical study.
(^{a}): Indicates taxa for which probes were used in the experiments to correlate bacterial cell counts and picomolar levels of complementary sequences.

Hybridization Using Oligonucleotide Probes

Before hybridization, the membranes were pre-wet in 2×SSC. The membranes were prehybridized in 35 ml of a solution containing 50% formamide, 5×SSC, 1% casein (Sigma, St Louis Mo.), 5×Denhardt's reagent (Maniatis, T., Molecular Cloning: A Laboratory Manual. Cold Spring Harbor, N.Y.: Cold Spring Harbor Laboratory (1982)), 25 mM sodium phosphate (pH 6.5) and 0.5 mg/ml yeast RNA (Boehringer Mannheim). The solution was placed into a plastic hybridization bag containing the membrane. The sealed bag was incubated at 42° C. for at least 90 min. Each membrane with fixed TNA was placed in a Miniblotter 45 (Immunetics, Cambridge Mass.), with the “lanes” of TNA at 90° to the channels of the device. A 30×45 “checkerboard” pattern was produced

Probes were diluted in a proprietary hybridization buffer (UltraHyb Oligo buffer, Ambion, Austin, Tex.). The final concentration of the different probes in each lane in the Miniblotter 45 varied from 2 to 60 μM. The digoxigenin-labeled oligonucleotide probes, diluted in UltraHyb Oligo buffer, were placed in individual lanes of the Miniblotter. Empty lanes were filled with hybridization solution. The apparatus was placed in a plastic bag and the membranes were hybridized at 42° C. for 1 hr and 20 minutes. Membranes were washed on a rotator with 250 ml of sterile 2×SSC 0.5% SDS at 37° C. for 1 hr. To detect hybrids, membranes were blocked in a maleic acid buffer containing 10% casein and then incubated with a 1:2,500 dilution of anti-digoxigenin antibody conjugated with alkaline phosphatase (Roche, Indianapolis, Ind.). The membrane and the solution were placed in a sealed hybridization bag and kept on a rotator for 30 min. The membranes were rinsed with maleic acid buffer for 5 min to remove excess antibody and then washed 3 times with that buffer for 15 min each time. The membranes were washed with 200 ml “buffer 3” (0.04% MgCl₂ and 2.1% diethanolamine (pH 9.5), equal volumes) for 5 min. Finally, 1 ml of a chemiluminescent substrate (CDP Star, Tropix, Bedford, Mass.) was diluted in 4 ml of “buffer 3”. The final solution was deposited onto the membrane surface. The membrane was exposed to an X-ray film in a radiograph cassette for 60 min. The films were scanned and signals intensities of the samples and the standards were measured using Total Lab software (NonLinear USA, Durham, N.C.). Signals were converted to absolute counts by comparison with standards on the membrane. Failure to detect a signal was recorded as zero.

Standards for the Oligonucleotide Probes

The standards for detection using oligonucleotide probes were a mixture of sequences complementary to the oligonucleotide probes designed to detect both the cultivable and as yet uncultivated taxa. The “complementary” sequences were synthesized by SigmaGenosys (Woodlands, Tex.). The final mixtures of standards had 0.004 or 0.04 μM of each sequence. 90 μl of 2% glutaraldehyde and 910 μl of 6×SSC were added to the standards. The final solutions of the standards were deposited as the last 2 lanes of each membrane.

Relation of Complementary Sequence Concentration to Bacterial Counts

It was estimated that 0.004 and 0.04 pM of complementary probe sequence would approximate 10⁵ and 10⁶ bacterial cells respectively. Since bacterial cells were not available from uncultivated taxa, cultivated bacterial species were employed to test this estimate (Table 3). Signals were compared from samples containing 10 ng of TNA, 10⁶ bacterial cells and 0.04 pM of oligonucleotide sequences. These targets were hybridized with probes to the species and the intensities of the signals were compared.

Determination of Probe Sensitivity

Bacterial suspensions were prepared from pure cultures. Upon harvesting, the cell density of each species was adjusted to OD₆₀₀=1. 10⁸ cells of each species were pipetted into a microcentrifuge tube. The resulting suspension was serially 10-fold diluted and TNA from each dilution was individually extracted using the Masterpure RNA purification kit. The samples were quantified as described above.

Determination of Probe Specificity

To determine the specificity of the probes, 10 ng of TNA from 96 different bacterial species commonly found in the oral cavity, as well as 20 “complementary” sequences for the uncultivated phylotypes were laid on the nylon membranes using a Minislot. The membranes were then “probed” in a checkerboard format using all the oligonucleotide probes.

Effect of Multiple Species in Different Levels on Signal Detection

To determine the influence of the presence of different numbers of cells from multiple species on quantification of target species, a series of bacterial mixtures were prepared. Suspensions containing 10³ to 10⁶ cells from a range of bacterial species were prepared. TNAs were extracted from these mixtures and laid on a nylon membrane and quantified as described above.

Pilot Clinical Study

In order to assess the feasibility of the proposed method, a small pilot study was conducted. Eight periodontally healthy individuals and eleven periodontitis subjects were selected for study. Periodontally healthy individuals had more than 24 teeth and presented no sites with pocket depth and/or attachment level greater than 3 mm. Periodontitis patients had at least 20 teeth, at least 5% of sites (measured at up to 168 sites) with pocket depth of 5 mm or greater and attachment level greater than 3 mm.

Subjects included in the study had no systemic condition that might influence periodontal disease or dental treatment. Upon enrollment in the study, all subjects signed an informed consent. The informed consent and study protocol were approved by the Institutional Review Board (IRB) at The Forsyth Institute, where all of the subjects were monitored and sampled.

Samples were taken from two randomly selected contralateral quadrants in each subject, providing a total of 14 samples per subject. After removal of supragingival plaque, subgingival plaque samples were individually taken from each mesio-buccal site in the selected quadrants using sterile 11/12 Gracey curettes. Each sample was placed in a microcentrifuge tube containing 100 μl of RNAse-free TE buffer. Samples were kept at −80° C. until TNA extraction.

Data Analysis

Signals were converted to approximate “counts” by comparison to the standards on each membrane. The “counts” were computed by estimating that 0.04 pM of target sequences in the standard was equivalent to approximately 10⁶ cells and that 0.004 pM target sequences approximated 10⁵ cells. Absence of signal detection was recorded as zero. In clinical samples, “counts” for each taxon were averaged within a subject and then across subjects in the periodontitis and periodontally healthy groups separately. Significance of differences between subject groups was determined using the Mann Whitney test. In this pilot investigation, no effort was made to correct for multiple comparisons.

### Example 2

### Objective: to Identify Relevant (i.e., Common and in High Numbers) Uncultivated/Unrecognized Bacterial Species in Periodontal Health and Disease

The objective of the present work is to determine which of the probes to uncultivated/unrecognized taxa will detect taxa that are common and in high numbers in subgingival biofilm samples. The long term objective of this work is to identify and isolate in pure culture uncultivated and unrecognized bacterial species associated with periodontal health and disease.

The methods employed herein are similar to those described in example 1. The experiments described below were proposed to be performed in two stages. In Stage 1, samples from 8 periodontally healthy subjects and 8 periodontitis patients would be analyzed for their content of 140 uncultivated/unrecognized taxa using the RNA-oligonucleotide quantification technique (ROQT). Test taxa would be selected based on preliminary data generated in a recently completed study (NIH 5R01DE017400-03; PI A. Haffajee). Probes that fail to detect a taxon/taxa in at least 10% of the samples would not be further employed in Stage 2. Then, in Stage 2, 8 additional periodontally healthy subjects and 8 additional periodontitis patients would be enrolled, have subgingival samples collected and analyzed for the prevalence and levels of the most common and abundant taxa identified in Stage 1. Again, probes that fail to detect a taxon/taxa in at least 10% of those samples would not be further employed in SA2.

Results from Stage 1:

8 periodontally healthy and 8 periodontitis subjects were enrolled in the study. Table 4 shows their demographic and clinical characteristics. Subgingival biofilm samples were collected and their total nucleic acids (TNA) were extracted from each sample individually and laid on a nylon membrane for microbial analysis using ROQT.

TABLE 4
Demographic and clinical characteristics
of the study population

| | Healthy | | Periodontitis | | |
|---|---|---|---|---|---|
| | 8 | | 8 | | MW |
| N | Mean | SD | Mean | SD | p |
| % Males | 0.4 | 0.5 | 0.6 | 0.5 | 0.3329 |
| Age (years) | 33.8 | 8.6 | 46.3 | 11.4 | 0.0263 |
| PD (mm) | 1.8 | 0.3 | 3.2 | 0.6 | 0.0008 |
| CAL (mm) | 1.1 | 0.5 | 3.4 | 1.0 | 0.0008 |
| BOP (0/1) | 0.1 | 0.1 | 0.5 | 0.3 | 0.0008 |
| PI (0/1) | 0.4 | 0.3 | 0.8 | 0.4 | 0.0157 |
| SUP (0/1) | 0.0 | 0.0 | 0.0 | 0.0 | 0.0378 |
| RED (0/1) | 0.1 | 0.1 | 0.6 | 0.4 | 0.0011 |

PD = pocket depth;
CAL = clinical attachment level;
BOP = bleeding on probing;
PI = plaque index;
SUP = suppuration;
RED = redness
MW = Mann-Whitney Test

As mentioned above, the taxa and probes employed in the present study were selected based on data from a recently completed project. They are presented in Table 5, where they are ordered according to decreasing prevalence of those taxa in periodontitis subjects. In that study, subgingival biofilm samples were collected from 16 periodontally healthy subject and 28 periodontitis patients and analyzed using the Human Oral Microbial Identification Microarray (HOMIM).

All probe sequences selected to be employed in Stage 1 of SA1 (and throughout this study) derived from the probe panel currently employed in the HOMIM. Probes selection was performed based on one core criterion: they should target only uncultivated or cultivated but unrecognized taxa. There are a number of HOMIM sequences that target two or more taxa, including cultivated species. Therefore, we aimed at avoiding sequences that would identify recognized (named) cultivated bacterial species and uncultivated/unrecognized taxa simultaneously. By taking this approach, when a signal was observed, we would not question whether the taxon/taxa identified was truly an uncultivated/unrecognized phylotype. Out of the more than 300 sequences in the HOMIM panel, only 90 of them could fit this criterion. These probes target a total of 124 phylotypes: 78 uncultivated and 46 cultivated but unrecognized (Table 5).

TABLE 5
Test Taxa selected for microbial analysis:

| Probes and Test Uncultivated/Unrecognized Taxa | UU | UC |
|---|---|---|
| Fusobacterium sp clones BB002 and FB074_ot210_220 | 210, 220 | |
| Synergistetes[G-3] sp clone BH017_ot360 | 360 | |
| Capnocytophaga sp clone_X066_ot335 | | 335 |
| Streptococcus sp strains Hans H6 and 7A_ot070_071 | | 070, 071 |
| Acidaminococcaceae[G-1] sp clone C3MLM071 and sp strains FYA47 and GAA14_ot155 | | 155 |
| Peptostreptococcaceae[13][G-1]sp clone DA014_ot113 | 113 | |
| Bacteroidetes[G-2] sp clone AU126_ot274 | | 274 |
| Desulfobulbus sp clone_R004_ot041 | 41 | |
| Peptostreptococcaceae[11][G-4] sp clone MCE10_174 and sp strain PUS9.170_ot103_369 | 103, 369 | |
| Selenomonas sp clones DD020 and P4PA_145_ot134_442 | 134, 442 | |
| Selenomonas sp clones DS071 and EW084_ot138_146 | | 138, 146 |
| Lachnospiraceae[G-6] sp clone BB124 ot080 | 80 | |
| TM7[G-1] sp clones AH040 and BS003_ot346 | 346, 349 | |
| Haemophilus sp clone BJ095_ot036 | | 36 |
| Actinomyces sp clone EP053_ot177 | 177 | |
| Treponema sp clones AT039 and AU076_ot237_242 | 237, 242 | |
| Synergistetes[G-3] Cluster I_ot363_453_452 | 363, 453, 452 | |
| Treponema Cluster II_ot254_256_508_517 | 254, 256, 508, 517 | |
| Haemophilus sp clone BJ021_ot035 | 35 | |
| Acidaminococcaceae[G-1] sp clones DM071 and EZ011_ot135_148 | 135, 148 | |
| Peptostreptococcaceae[11][G-7] sp clone A03MT_ot106 | | 106 |
| Bacteroidetes[G-1] sp clone_X083_ot272 | | 272 |
| Eubacterium sp clone BB142_ot081 | 81 | |
| TM7[G-5] sp clones_I025 and P4PB_40_ot356_437 | 356, 437 | |
| Bergeyella sp clone AK152_ot322 | 322 | |
| Synergistetes[G-3] sp clone BH007_ot359 | 359 | |
| TM7[G-1] sp clone BE109 and TM7[G-2] sp clone BU080_ot347_350 | 347, 350 | |
| Megasphaera sp clone CS025_ot123 | 123 | |
| Actinomyces sp clone EP005_ot175 | | 175 |
| Tannerella sp clone BU063_ot286 | 286 | |
| Veillonella sp clones FO009 and HB016_ot780 | | 780 |
| Lachnospiraceae[G-3] sp clone DO008_ot096 | | 96 |
| Oribacterium sp clones AO068 and MCE9_31_ot078_372 | | 78, 372 |
| Synergistetes[G-3] sp clones BH017 and D084 and JV006_ot360_362_453 | 360, 362, 453 | |
| Selenomonas sp clone CS024_ot133 | | 133 |
| Eubacterium sp clone 7 69_ot846 | | 846 |
| Treponema Cluster III ot256_508_517_518 | 256, 508, 517, 518 | |
| Acidaminococcaceae[G-1] sp clones_K024 and P2PAS_80 and CS015_ot132_150 | 132, 150 | |
| Actinomyces sp clone AP064_ot170 | | 170 |
| Selenomonas Cluster sp clones DO042 and FT050 and GI064_ot136_149_478 | 478 | 136, 149 |
| Lachnospiraceae[G-4] sp clone D0016_ot097 | 97 | |
| Synergistetes[G-3] sp clone W090_ot363 | | 362 |
| Capnocytophaga sp clone DS022_ot332 | | 332 |
| Lachnospiraceae[G-3] sp clone D0008_ot096 | | 96 |
| Leptotrichia sp clone DR011_ot215 | 215 | |
| Treponema sp clone_T021_ot231 | 231 | |
| Treponema sp strain Smibert-3 D36_ot257 | | 257 |
| Actinomyces sp clone AG004_ot169 | | 169 |
| Actinomyces sp clone IP073_ot448 | 448 | |
| Actinomyces sp strain B27SC_ot178 | | 178 |
| Atopobium sp clone C3MLM018_ot416 | 416 | |
| Bacteroidetes[G-3] sp clone DA065 and sp strain MB4_G15_ot281_365 | 281, 365 | |
| Capnocytophaga sp clone BB167_ot326 | | 326 |
| Prevotella sp clone BI027_ot299 | | 299 |
| Prevotella sp clone DO039_ot308 | 308 | |
| Prevotella sp clone DO045_ot309 | 309 | |
| Prevotella sp strains C3MKM081 and TFI B31FD_ot317 | | 317 |
| Neisseria sp strain B33KA_ot020 | | 20 |
| Acidaminococcaceae[G-2] sp clones CS002 and C5AKM062_ot131 | 131 | |
| Selenomonas sp clone DS051_ot137 | | 137 |
| Clostridiales[F-2][G-1] sp clone_F058_ot075 | 75 | |
| Clostridiales[F-2][G-2] sp clone BU014_ot085 | 85 | |
| Leptotrichia sp clone C3MKM102_ot417 | 417 | |
| Leptotrichia sp clones C3MKM102 and GT018_ot417_462 | 417, 462 | |
| Synergistetes[G-3] sp clone BB062_ot358 | 358 | |
| Actinobaculum sp clone EL030 and sp strain P2P_19_ot183 | | 183 |
| Capnocytophaga sp clone AA032_ot324 | | 324 |
| Capnocytophaga sp clone_X089_ot336 | | 336 |
| Porphyromonas sp clone DP023_ot279 | | 279 |
| Prevotella sp clone AH125 and sp strain C3MLM058_ot292_300 | 292, 300 | |
| Prevotella sp clones AH125 and AU120_ot292 | 292 | |
| Prevotella sp clone DR022_ot310 | 310 | |
| Prevotella sp strain E7_34E1_ot376 | | 376 |
| Prevotella Cluster III_ot306_310_313 | 310, 313 | 306 |
| Prevotella Cluster sp clones HF050 and ID019 and IK062_ot473_474 | 474 | 473 |
| Burkholderia sp clone AK168_ot406 | 406 | |
| Neisseria Cluster IV ot009_014_015_016 | 15 | 14, 16 |
| Rhodocyclus sp strain A08KA_ot028 | | 28 |
| Acidaminococcaceae[G-1] sp clone DM071_ot135 | 135 | |
| Acidaminococcaceae[G-1] sp clones EW079 and J5031_ot145_483 | 145, 483 | |
| Acidaminococcaceae[G-1] sp clones AU096 and_K024 and P2PAS_80_ot129_150 | 150 | 129 |
| Selenomonas sp clones FT050 and IK004_ot149_481 | | 149, 481 |
| Selenomonas sp clone EW084_ot146 | | 146 |
| Oribacterium sp clone AO068_ot078 | | 78 |
| Lactobacillus sp clone HT070_ot461 | 461 | |
| SR1[G-1] sp_X112_ot345 | 345 | |
| Synergistetes[G-3] sp clone D084_ot362 | 362 | |
| Treponema sp clone_U008A_ot251 | 251 | |
| Actinomyces sp clones IP073 and IO076_ot446_448 | 446, 448 | |

UU: Uncultivated and unrecognized taxa;
UC: Unrecognized cultivated taxa.
Targeted taxa are indicated by genus and clones.
The presented nomenclature follows the taxonomy proposed in the Human Oral Microbial Database (HOMD) guidelines (www.homd.org).

Thus far, subgingival samples have been analyzed for the prevalence and levels of 75 taxa (49 uncultivated; 26 cultivated but unrecognized) (Table 6), using 51 oligonucleotide probes. Prior to analysis, all probes were tested against standards and individual targets containing known pM amounts of the reverse complement sequence. All clinical samples were analyzed individually. Standards for quantification were included in each assay. They comprised 0.004, 0.04, 0.16 and 0.32 picomolars (pM) of sequences that represented the reverse complement of each of the probe sequences. Quantification of individual taxa was performed by converting signal intensity of the TNA-probe hybrids, based on standard curves. Failure to detect a signal was recorded as zero. Data were computed as levels of taxa as well as prevalence (% of positive sites) in each of the clinical groups separately. Data were averaged across all samples within each clinical group. Significance of differences in levels between groups was sought using the Mann-Whitney test.

Table 6 shows the mean prevalence of the 75 taxa evaluated using 51 oligonucleotide probes thus far. Probes are presented in decreasing order of mean prevalence in samples from periodontitis patients. It can be observed that Synergistetes_OT_{—}359, Selenomonas_OT_{—}134_{—}442, TM7_OT_{—}346_{—}349, Capnocytophaga_OT_{—}335, Haemophilus_OT_{—}035, Synergistetes_OT_{—}363_{—}453_{—}452, Treponema_OT_{—}256_{—}508_{—}517, Actinomyces_OT_{—}177 and Desulfobulbus_OT_{—}041 are more frequently detected in sites from periodontitis patients than in sites from periodontally healthy individuals. They were present in at least 30% of the sites examined and thus reached the 10% cut off established as a criterion from inclusion in the next stage of the study. Several additional taxa are also more frequently detected in disease than in health and often times the magnitude of this difference reached statistical significance (p<0.05).

TABLE 6
Prevalence of 75 test taxa (% sites positive)

| N | Healthy | | Periodontitis | | MW |
|---|---|---|---|---|---|
| Taxa | 8 Mean % | SD | 8 Mean % | SD | p |
| Synergistetes_ot_359 | 38% | 49% | 45% | 50% | 0.4564 |
| Selenomonas_ot_134_442 | 27% | 45% | 38% | 49% | 0.2191 |
| TM7_ot_346_349 | 20% | 40% | 34% | 48% | 0.0958 |
| Capnocytophaga_ot_335 | 14% | 35% | 34% | 48% | 0.0175 |
| Haemophilus_ot_035 | 22% | 42% | 34% | 48% | 0.1785 |
| Synergistetes_ot_363_453_452 | 14% | 35% | 32% | 47% | 0.0304 |
| Treponema_ot_256_508_517 | 0% | 0% | 31% | 47% | 0.0000 |
| Actinomyces_ot_177 | 18% | 39% | 30% | 46% | 0.1434 |
| Desulfobulbus_ot_041 | 2% | 13% | 30% | 46% | 0.0001 |
| Bacteroidetes_ot_274 | 11% | 31% | 28% | 45% | 0.0238 |
| Actinomyces_ot_448 | 30% | 46% | 27% | 45% | 0.6839 |
| Lachnospiracea_ot_096 | 13% | 34% | 26% | 45% | 0.0737 |
| Synergistetes_ot_360_362_453 | 0% | 0% | 23% | 42% | 0.0002 |
| Haemphilus_ot_036 | 2% | 13% | 22% | 42% | 0.0011 |
| Synergistetes_ot_360 | 2% | 13% | 22% | 42% | 0.0011 |
| Capnocytophaga_ot_332 | 14% | 35% | 22% | 42% | 0.3012 |
| Peptostreptococcacea_ot_106 | 2% | 13% | 21% | 41% | 0.0018 |
| Peptostreptococcacea_ot_113 | 4% | 19% | 20% | 40% | 0.0080 |
| Actinomyces_ot_169 | 19% | 39% | 19% | 39% | 0.9899 |
| Selenomonas_ot_138_146 | 7% | 26% | 18% | 39% | 0.0904 |
| Bergeyella_ot_322 | 8% | 27% | 18% | 39% | 0.1213 |
| Tannerella_ot_286 | 4% | 19% | 17% | 38% | 0.0208 |
| Lachnospiracea_ot_080 | 2% | 13% | 16% | 37% | 0.0091 |
| Synergistetes_ot_363 | 8% | 27% | 16% | 37% | 0.1941 |
| Actinomyces_ot_170 | 22% | 42% | 16% | 37% | 0.4193 |
| Veillonella_ot_780 | 0% | 0% | 15% | 36% | 0.0026 |
| Actinomyces_ot_175 | 2% | 13% | 15% | 36% | 0.0130 |
| Peptostreptococcacea_ot_103_369 | 2% | 13% | 14% | 35% | 0.0180 |
| Oribacterium_ot_078_372 | 0% | 0% | 13% | 34% | 0.0051 |
| Megasphaera_ot_123 | 0% | 0% | 13% | 34% | 0.0060 |
| TM7_ot_347_350 | 9% | 29% | 13% | 34% | 0.4760 |
| Eubacterium_ot_081 | 0% | 0% | 13% | 34% | 0.0055 |
| Bacteroidetes_ot_272 | 0% | 0% | 13% | 34% | 0.0055 |
| Streptococcus_ot_070_071 | 0% | 0% | 12% | 33% | 0.0079 |
| Atopobium_ot_416 | 12% | 32% | 12% | 33% | 0.9718 |
| Actinomyces_ot_178 | 13% | 34% | 12% | 33% | 0.8258 |
| Treponema_ot_257 | 6% | 23% | 12% | 33% | 0.2438 |
| Leptotrichia_ot_215 | 7% | 26% | 12% | 33% | 0.4493 |
| Lachnospiracea_ot_096 | 8% | 27% | 12% | 33% | 0.4478 |
| Selenomonas_ot_136_149_478 | 6% | 24% | 12% | 33% | 0.2969 |
| TM7_ot_356_437 | 0% | 0% | 11% | 32% | 0.0121 |
| Acidaminococcacea_ot_135_148 | 0% | 0% | 11% | 32% | 0.0106 |
| Treponema_ot_237_242 | 2% | 13% | 10% | 30% | 0.0690 |
| Treponema_ot_231 | 4% | 19% | 10% | 30% | 0.2221 |
| Treponema_ot_256_508_517_518 | 4% | 20% | 10% | 31% | 0.2530 |
| Selenomonas_ot_133 | 7% | 26% | 10% | 30% | 0.6849 |
| Acidaminococcacea_ot_155 | 4% | 19% | 8% | 27% | 0.3270 |
| Fusobacterium_ot_210_220 | 2% | 13% | 6% | 24% | 0.2579 |
| Lachnospiracea_ot_097 | 6% | 25% | 6% | 24% | 0.9388 |
| Acidaminococcacea_ot_132_150 | 10% | 30% | 6% | 24% | 0.4814 |
| Eubacterium_ot_846 | 8% | 27% | 6% | 24% | 0.7373 |

Note:
Average of 7 samples per subject

Conversely, Acidaminococcacea_OT 155, Fusobacterium_OT_{—}210_{—}220, Lachnospiracea_OT_{—}097 and Eubacterium_OT_{—}846 are all present in less than 10% of samples in each clinical group; therefore these probes will not be employed in the next phase of the study.

Probes targeting cultivated taxa typically associated with periodontal health, such as Actinomyces naeslundii and Veillonella parvula, as well as species associated with periodontitis, such as Porphyromonas gingivalis and Fusobacterium nucleatum were used as “references”. Table 7 demonstrates the prevalence of those species in 3 different sets of samples from the study population. As expected, P. gingivalis was detected more frequently in sites from periodontitis subjects than in sites from periodontally healthy individuals. Interestingly, there are a number of uncultivated/unrecognized taxa listed in Table 6 were detected more frequently than P. gingivalis in samples from periodontitis subjects, suggesting a potential pathogenic role for those phylotypes.

TABLE 7
Prevalence of selected species

| Set 1 | Healthy | | Periodontitis | | |
|---|---|---|---|---|---|
| N | 8 | | 8 | | MW |
| Prevalence | Mean % | SD | Mean % | SD | p |
| P. gingivalis | 4% | 19% | 22% | 42% | 0.0041 |
| A. naeslundii | 9% | 29% | 18% | 39% | 0.1705 |
| F. nucleatum | 45% | 50% | 62% | 49% | 0.0753 |
| V. parvula | 7% | 26% | 26% | 44% | 0.0086 |
| Set 2 | Healthy | | Periodontitis | | |
| N | 8 | | 8 | | MW |
| Prevalence | Mean % | SD | Mean % | SD | p |
| P. gingivalis | 0% | 0% | 19% | 39% | 0.0008 |
| A. naeslundii | 5% | 23% | 17% | 38% | 0.0579 |
| F. nucleatum | 45% | 50% | 58% | 50% | 0.1501 |
| V. parvula | 2% | 13% | 15% | 36% | 0.0130 |
| Set 3 | Healthy | | Periodontitis | | |
| N | 8 | | 8 | | MW |
| Prevalence | Mean % | SD | Mean % | SD | p |
| P. gingivalis | 9% | 29% | 16% | 37% | 0.3013 |
| A. naeslundii | 27% | 45% | 14% | 35% | 0.0981 |
| F. nucleatum | 45% | 50% | 47% | 50% | 0.8566 |
| V. parvula | 15% | 36% | 14% | 35% | 0.8442 |

Note:
Average of 7 samples per subject in each set

Table 8 shows the mean levels of the 75 taxa evaluated using 51 oligonucleotide probes thus far. Probes are ordered according to decreasing mean levels in samples from periodontitis patients. It can be observed that Synergistetes_OT_{—}359, TM7_OT_{—}346_{—}349, Haemophilus_OT_{—}035, Synergistetes_OT_{—}363_{—}453_{—}452, Bacteroide tes_OT_{—}274 and Capnocytophaga_ot_{—}335 were highly abundant in samples from periodontitis patients, in comparison with samples from healthy individuals. Several additional taxa were also more numerous in disease than in health and often times the magnitude of this difference reached statistical significance (p<0.05). Conversely, several taxa were present at levels below 0.004 pM, which represents the lowest level of the standards employed in the assay.

TABLE 8
Levels of test taxa

| | Healthy | | Periodontitis | | |
|---|---|---|---|---|---|
| N | 8 | | 8 | | MW |
| Taxa | Mean (pM) | SD | Mean (pM) | SD | p |
| Synergistetes_ot_359 | 0.008 | 0.018 | 0.029 | 0.053 | 0.1208 |
| TM7_ot_346_349 | 0.012 | 0.039 | 0.023 | 0.056 | 0.0829 |
| Haemophilus_ot_035 | 0.003 | 0.006 | 0.02 | 0.040 | 0.0574 |
| Synergistetes_ot_363_453_452 | 0.002 | 0.007 | 0.018 | 0.043 | 0.0126 |
| Bacteroidetes_ot_274 | 0.003 | 0.014 | 0.018 | 0.041 | 0.0166 |
| Capnocytophaga_ot_335 | 0.003 | 0.009 | 0.017 | 0.037 | 0.0110 |
| Capnocytophaga_ot_332 | 0.001 | 0.004 | 0.015 | 0.037 | 0.1787 |
| Selenomonas_ot_134_442 | 0.006 | 0.017 | 0.013 | 0.025 | 0.1394 |
| Synergistetes_ot_360 | 0 | 0.002 | 0.009 | 0.024 | 0.0011 |
| Treponema_ot_256_508_517 | 0 | 0 | 0.009 | 0.022 | 0.0000 |
| Desulfobulbus_ot_041 | 0 | 0.001 | 0.008 | 0.022 | 0.0001 |
| Synergistetes_ot_360_362_453 | 0 | 0 | 0.008 | 0.020 | 0.0002 |
| Actinomyces_ot_448 | 0.016 | 0.052 | 0.008 | 0.021 | 0.6255 |
| Tannerella_ot_286 | 0 | 0.001 | 0.006 | 0.019 | 0.0169 |
| Lachnospiracea_ot_096 | 0.002 | 0.006 | 0.006 | 0.016 | 0.0547 |
| Actinomyces_ot_169 | 0.004 | 0.016 | 0.006 | 0.025 | 0.9727 |
| Leptotrichia_ot_215 | 0 | 0.001 | 0.006 | 0.022 | 0.3817 |
| Synergistetes_ot_363 | 0.001 | 0.004 | 0.006 | 0.019 | 0.1712 |
| Actinomyces_ot_177 | 0.01 | 0.033 | 0.005 | 0.010 | 0.2371 |
| Bergeyella_ot_322 | 0.002 | 0.007 | 0.005 | 0.014 | 0.1068 |
| Selenomonas_ot_136_149_478 | 0 | 0.002 | 0.005 | 0.025 | 0.2787 |
| Peptostreptococcacea_ot_106 | 0 | 0.001 | 0.004 | 0.010 | 0.0016 |
| Selenomonas_ot_138_146 | 0 | 0.001 | 0.003 | 0.008 | 0.0628 |
| Actinomyces_ot_170 | 0.004 | 0.012 | 0.003 | 0.009 | 0.4701 |
| Treponema_ot_256_508_517_518 | 0 | 0.001 | 0.003 | 0.011 | 0.2340 |
| Haemphilus_ot_036 | 0 | 0.001 | 0.002 | 0.005 | 0.0009 |
| Lachnospiracea_ot_080 | 0 | 0.001 | 0.002 | 0.005 | 0.0088 |
| Peptostreptococcacea_ot_103_369 | 0 | 0.001 | 0.002 | 0.006 | 0.0181 |
| Peptostreptococcacea_ot_113 | 0 | 0.002 | 0.002 | 0.006 | 0.0081 |
| TM7_ot347_350 | 0.002 | 0.007 | 0.002 | 0.006 | 0.4948 |
| Actinomyces_ot_175 | 0 | 0.001 | 0.002 | 0.005 | 0.0113 |
| TM7_ot356_437 | 0 | 0 | 0.002 | 0.005 | 0.0121 |
| Eubacterium_ot_081 | 0 | 0 | 0.002 | 0.004 | 0.0056 |
| Atopobium_ot_416 | 0.001 | 0.003 | 0.002 | 0.006 | 0.9150 |
| Actinomyces_ot_178 | 0.001 | 0.003 | 0.002 | 0.005 | 0.8669 |
| Lachnospiracea_ot_096 | 0.001 | 0.002 | 0.002 | 0.006 | 0.3930 |
| Treponema_ot_237_242 | 0 | 0.001 | 0.001 | 0.005 | 0.0633 |
| Acidaminococcacea_ot_155 | 0 | 0.002 | 0.001 | 0.005 | 0.3272 |
| Streptococcus_ot_070_071 | 0 | 0 | 0.001 | 0.004 | 0.0079 |
| Fusobacterium_ot_210_220 | 0 | 0.001 | 0.001 | 0.005 | 0.2501 |
| Oribacterium_ot_078_372 | 0 | 0 | 0.001 | 0.004 | 0.0052 |
| Veillonella_ot_780 | 0 | 0 | 0.001 | 0.004 | 0.0027 |
| Megasphaera_ot_123 | 0 | 0 | 0.001 | 0.003 | 0.0060 |
| Bacteroidetes_ot272 | 0 | 0 | 0.001 | 0.003 | 0.0055 |
| Acidaminococcacea_ot_135_148 | 0 | 0 | 0.001 | 0.002 | 0.0106 |
| Treponema_ot_257 | 0 | 0.001 | 0.001 | 0.005 | 0.2389 |
| Treponema_ot_231 | 0 | 0.002 | 0.001 | 0.006 | 0.2224 |
| Lachnospiracea_ot_097 | 0 | 0.002 | 0.001 | 0.003 | 0.9853 |
| Acidaminococcacea_ot_132_150 | 0.001 | 0.002 | 0.001 | 0.003 | 0.5100 |
| Eubacterium_ot_846 | 0 | 0.001 | 0.001 | 0.004 | 0.8091 |
| Selenomonas_ot_133 | 0.001 | 0.003 | 0.001 | 0.003 | 0.7046 |

Average of 7 sites/patient

Table 9 shows the levels of health- and periodontitis-associated bacterial species in 3 different sets of samples from the study population. As expected, P. gingivalis and F. nucleatum were detected in higher levels in sites from periodontitis subjects. Interestingly, a number of uncultivated/unrecognized taxa listed in Table 5 were detected in even higher quantities than P. gingivalis in samples from disease individuals, suggesting a potential role for those phylotypes in the pathogenesis of periodontitis.

TABLE 9
Levels of selected species

| | Healthy | | Periodontitis | | |
|---|---|---|---|---|---|
| N | 8 | | 8 | | MW |
| Set 1 | Mean | SD | Mean | SD | p |
| P. gingivalis | 0.000 | 0.001 | 0.008 | 0.028 | 0.0036 |
| A. naeslundii | 0.002 | 0.006 | 0.002 | 0.005 | 0.2221 |
| F. nucleatum | 0.027 | 0.050 | 0.054 | 0.071 | 0.0285 |
| V. parvula | 0.001 | 0.006 | 0.003 | 0.008 | 0.0125 |
| | Healthy | | Periodontitis | | |
| N | 8 | | 8 | | MW |
| Set 2 | Mean | SD | Mean | SD | p |
| P. gingivalis | 0.000 | 0.000 | 0.006 | 0.017 | 0.0008 |
| A. naeslundii | 0.000 | 0.001 | 0.002 | 0.006 | 0.0500 |
| F. nucleatum | 0.027 | 0.058 | 0.068 | 0.120 | 0.0748 |
| V. parvula | 0.000 | 0.001 | 0.005 | 0.015 | 0.0113 |
| | Healthy | | Periodontitis | | |
| N | 8 | | 8 | | MW |
| Set 3 | Mean | SD | Mean | SD | p |
| P. gingivalis | 0.001 | 0.004 | 0.009 | 0.034 | 0.2435 |
| A. naeslundii | 0.007 | 0.016 | 0.003 | 0.010 | 0.1010 |
| F. nucleatum | 0.051 | 0.113 | 0.074 | 0.129 | 0.6685 |
| V. parvula | 0.003 | 0.009 | 0.005 | 0.014 | 0.9781 |

Average of 7 samples per subject per set

Overall, the data presented above suggest that certain uncultivated/unrecognized taxa might be involved in initiation and progression of periodontitis, based on their high prevalence and levels in diseased individuals, in comparison with healthy subjects. Thus, they merit further pursuit, regarding attempts of isolation in pure culture and characterization. They include Synergistetes_OT_{—}359, Selenomonas_OT_{—}134_{—}442, TM7 of 346_{—}349, Capnocytophaga_OT_{—}335, Haemophilus_OT_{—}035, Synergistetes_OT_{—}363_{—}453_{—}452, Treponema_OT_{—}256_{—}508_{—}517, Actinomyces_OT_{—}177, Desulfobulbus_OT_{—041} and Bacteroidetes_OT_{—}274 (Tables 6 and 8). In addition, it also became clear that other taxa are only rarely detected in subgingival biofilms and/or seem to be present in low levels. These phylotypes might represent transient taxa or simply “bystanders” that do not have a significant role in the disease process, but may act as a distraction in our search for microorganisms that are truly relevant for the pathogenesis of periodontitis. In this dataset, such taxa are represented by Selenomonas_OT_{—}133, Acidaminococcacea_OT_{—}155, Fusobacterium_OT_{—}210_{—}220, Lachnospiracea_OT_{—}097, Acidaminococcacea_OT_{—}132_{—}150 and Eubacterium_OT_{—}846 (Tables 6 and 8).

We are currently performing the microbial analysis of subgingival samples using 34 additional probes that target 50 taxa (30 uncultivated and 20 cultivated). This step should be completed within the next few weeks. Once these assays are finalized, a more streamlined probe panel will be available for use in Stage 2.

Results from Stage 2:

Subgingival biofilm samples have been collected from 6 additional periodontally healthy and 2 additional periodontitis subjects. We are currently extracting total nucleic acids from these samples. They will be evaluated for the prevalence and levels of the most abundant uncultivated and cultivated but unrecognized taxa determined upon completion of Stage 1. This portion of the study is currently ongoing and 7 more study participants will be recruited for sample collection and analysis (2 periodontally healthy subject and 6 periodontitis individuals. At the end of this step, a final panel of 40 probes will be defined and employed.

2) Isolate in Pure Culture Cultivable but as Yet Unrecognized Prominent Taxa.

Once a set of oligonucleotide probes to 40 prominent unrecognized taxa have been selected, then we will use these probes to identify those that are cultivable using relatively standard cultural methods. The approach will be to use the oligonucleotide probes to the selected 40 prominent uncultivable/unrecognized taxa to screen isolates from subgingival biofilm samples grown on a range of isolation media and in different atmospheres.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for identifying and quantifying one or more microorganisms in a sample from an individual, wherein the method comprises:
a) extracting the total nucleic acid (TNA) from the sample;
b) contacting TNA from the sample with one or more probes that correlate to a microorganism to be quantified, under conditions suitable for hybridization to thereby form a complex; and
c) detecting the amount of the complex,
wherein the amount of the complex correlates with the amount of TNA of one or more microorganism in the sample.

2. The method of claim 1, wherein cultivated microorganisms, uncultivated microorganisms, or both are identified and quantified.

3. The method of claim 1, wherein a plurality of microorganisms are quantified.

4. The method of claim 2, wherein detecting nucleic acid of the microorganism includes detecting 16S rRNA of the microorganism.

5. The method of claim 1, wherein the sample from the individual is obtained from the group consisting of the oral cavity, sinus, esophagus, respiratory tract, lungs, sputum, pharynx, eustachian tube, middle ear, vagina, blood, pus, spinal fluid, and gastrointestinal tract.

6. The method of claim 1, further comprising labeling the nucleic acid molecules of the sample with a detectable label.

7. The method of claim 6, wherein the detectable label is selected from the group consisting of digoxigenin, fluorescent dyes, streptavidin conjugate, magnetic beads, dendrimers, radiolabels, enzymes, colorimetric labels, nanoparticles, and nanocrystals.

8. The method of claim 1, wherein the nucleic acid are bound to a solid support.

9. The method of claim 8, wherein the solid support is selected from the group consisting of nylon membrane, glass, silica chips, polymer, plastic, ceramic, metal, and optical fiber.

10. A method for assisting in the diagnosis an individual having a disease or condition, the method comprises:
a) determining the amount TNA of one or more cultivated or uncultivated microorganisms in a sample from the individual,
wherein an amount of TNA of a microorganism correlates to a disease state or a healthy state.

11. The method of claim 10, wherein the amount of TNA between about 10³ and about 10⁶ of a given taxon correlates to a disease state or a healthy state.

12. A method for diagnosing an individual having a periodontal disease, the method comprises:
a) determining an amount of TNA of one or more cultivated or uncultivated microorganisms in a sample from the individual,
wherein the amount of TNA between about 10³ and about 10⁶ of one or more of microorganism correlates to a disease state or a healthy state, and an amount less than 10³ does not correlate to a disease state or a healthy state.

13. The method of claim 12, wherein a periodontal disease state is associated with the amount of TNA between about 10³ and about 10⁶ of Fusobacterium nucleatum. ss polymorphum, Actinomyces gerencseriae, Mitsuokella sp OT_{—}131, Prevotella sp OT 306, Porphyromonas gingivalis, Peptostreptococcus strains BS044 or CK035, Desulfobulbus sp OT 041, Synergistetes_OT_{—}359, Selenomonas_OT_{—}134_{—}442, TM7_OT_{—}346_{—}349, Capnocytophaga_OT_{—}335, Haemophilus_OT_{—}035, Synergistetes_OT_{—}363_{—}453_{—}452, Treponema_OT_{—}256_{—}508_{—}517, Actinomyces_OT_{—}177, and Bacteroidetes_OT_{—}274.

14. A method of monitoring treatment for a disease associated with one or more bacteria; the method comprises:
a) determining the amount of TNA of one or more cultivated or uncultivated microorganisms in a sample from the individual at one or more time points,
b) comparing the amount of TNA of the one or more cultivated or uncultivated microorganism from step a) to a standard;
wherein the amount of TNA between about 10³ and about 10⁶ of one or more of microorganism correlates to the amount of the one or more microorganism present in the sample; and
a decrease in bacteria associated with disease indicates successful treatment and an increase in bacterial associated with said disease indicates unsuccessful treatment.

15. A method of determining the efficacy of an oral composition; the method comprises:
a) contacting the oral composition with a sample from an individual;
b) determining the amount of TNA of one or more cultivated or uncultivated microorganisms in a sample from the individual before and after step a); and
c) comparing the amount of TNA of the one or more cultivated or uncultivated microorganism obtained from step b);
wherein the amount of TNA between about 10³ and about 10⁶ of one or more of microorganism correlates to the amount of the microorganism in the sample; and a decrease in the amount of TNA in the sample indicates an effective oral composition; and an increase or no change in the amount of TNA in the sample indicates an ineffective oral composition.

16. A method for identifying one or more microorganisms associated with a disease or condition in an individual, the method comprises:
a) determining the amount TNA of one or more cultivated or uncultivated microorganisms in a sample from the individual,
wherein the amount of the complex correlates with the amount of TNA of one or more microorganism in the sample.

17. The method of claim 16, wherein the amount of TNA that correlates with one or more microorganisms is present in an amount between about 10³ and about 10⁶ in the sample.


SEQUENCE LISTING (585 sequences)

SEQ ID NO: 1 - DNA, 19 bp, Actinobacillus actinomycetemcomitans
tctctgagttcttcttcgg

SEQ ID NO: 2 - DNA, 20 bp, Actinobacillus actinomycetemcomitans
accaaccttcctcaataccg

SEQ ID NO: 3 - DNA, 18 bp, Actinobaculum
  misc_feature: sp. EL030
gagttaagccccgcattt

SEQ ID NO: 4 - DNA, 17 bp, Actinobaculum
  misc_feature: sp. EL030
gaagaaggcagggtact

SEQ ID NO: 5 - DNA, 18 bp, Actinomyces georgiae
atgcgaggaccagtgaat

SEQ ID NO: 6 - DNA, 16 bp, Actinomyces georgiae
aaagccaggcttcagc

SEQ ID NO: 7 - DNA, 16 bp, Actinomyces gerensceriae
accccagaagcccgtt

SEQ ID NO: 8 - DNA, 18 bp, Actinomyces gerensceriae
acagacccagaaacatcc

SEQ ID NO: 9 - DNA, 17 bp, Actinomyces naeslundii
agaccggcacatgtcaa

SEQ ID NO: 10 - DNA, 18 bp, Actinomyces naeslundii
tcatccagaaccagcaag

SEQ ID NO: 11 - DNA, 17 bp, Actinomyces odontolyticus
acaaccttgagttgcgg

SEQ ID NO: 12 - DNA, 19 bp, Actinomyces odontolyticus
ccatgcgaagatcagtgaa

SEQ ID NO: 13 - DNA, 17 bp, Actinomyces
  misc_feature: sp. AP064
acacccaccacaaagga

SEQ ID NO: 14 - DNA, 17 bp, Actinomyces
  misc_feature: sp. strain B19SC
cagagccagaatatccc

SEQ ID NO: 15 - DNA, 18 bp, Actinomyces
  misc_feature: sp. strain B19SC
gaatcgccgacatgtcaa

SEQ ID NO: 16 - DNA, 18 bp, Actinomyces
  misc_feature: sp. strain B27SC
tgcggagaccagtgaata

SEQ ID NO: 17 - DNA, 14 bp, Actinomyces
  misc_feature: sp. strain B27SC
actcacccacccag

SEQ ID NO: 18 - DNA, 18 bp, Actinomyces
  misc_feature: sp. EP005
agcaaaaaccggtccctt

SEQ ID NO: 19 - DNA, 18 bp, Actinomyces
  misc_feature: sp. EP011
ccacaaaaaaggagcagg

SEQ ID NO: 20 - DNA, 16 bp, Actinomyces
  misc_feature: sp. EP011
cacaagggaggaaacc

SEQ ID NO: 21 - DNA, 18 bp, Actinomyces
  misc_feature: sp. EP053
cacttcacagtgtcgcaa

SEQ ID NO: 22 - DNA, 18 bp, Actinomyces
  misc_feature: sp. EP053
aaccggtcgaatctttcc

SEQ ID NO: 23 - DNA, 16 bp, Actinomyces israelii
ggcacagccagaacac

SEQ ID NO: 24 - DNA, 17 bp, Actinomyces israelii
ttcttcaccggcgaaga

SEQ ID NO: 25 - DNA, 18 bp, Atopobium parvulum
ggctatcccaatgaaagg

SEQ ID NO: 26 - DNA, 18 bp, Atopobium parvulum
gcggaagtctcgaagtat

SEQ ID NO: 27 - DNA, 18 bp, Atopobium rimae
gccacattgaagtatcgg

SEQ ID NO: 28 - DNA, 18 bp, Atopobium rimae
ggaagacgtattctccca

SEQ ID NO: 29 - DNA, 18 bp, Atopobium
  misc_feature: sp. C019
gcggctcaagtgaagtat

SEQ ID NO: 30 - DNA, 19 bp, Tannerella forsythia
gaagaaagctctcactctc

SEQ ID NO: 31 - DNA, 18 bp, Tannerella forsythia
ttgcgggcaggttacata

SEQ ID NO: 32 - DNA, 18 bp, Bacteroidetes
  misc_feature: sp. _X083
gcagtaccaacagagtac

SEQ ID NO: 33 - DNA, 18 bp, Bacteroidetes
  misc_feature: sp. _X083
gcccatacatttgacagc

SEQ ID NO: 34 - DNA, 19 bp, Bacteroidetes
  misc_feature: sp. AU126
agatgcctcttccgtttac

SEQ ID NO: 35 - DNA, 19 bp, Bacteroidetes
  misc_feature: sp. AU126
acgtgtctcactttactcc

SEQ ID NO: 36 - DNA, 14 bp, Bifidobacterium
ggacgcgaccccat

SEQ ID NO: 37 - DNA, 18 bp, Bifidobacterium dentium
ccataccgacggatcttt

SEQ ID NO: 38 - DNA, 18 bp, Bifidobacterium
  misc_feature: sp. CX010
ccccatcatatggcacaa

SEQ ID NO: 39 - DNA, 18 bp, Bifidobacterium
  misc_feature: sp. CX010
agctatccccaaccatac

SEQ ID NO: 40 - DNA, 17 bp, Bifidobacterium
  misc_feature: sp. strain A32ED
cggatcggtcgggaaca

SEQ ID NO: 41 - DNA, 20 bp, Brevundimonas diminuta
attccgaaccaaaaggcacg

SEQ ID NO: 42 - DNA, 20 bp, Brevundimonas diminuta
tgggtttccaggcatgtcaa

SEQ ID NO: 43 - DNA, 18 bp, Bulledia extructa
  misc_feature: sequence also found in Solobacterium moorei
ccagagattatcccagtc

SEQ ID NO: 44 - DNA, 22 bp, Campylobacter concisus
ccctatctaacttatgtaagac

SEQ ID NO: 45 - DNA, 18 bp, Campylobacter concisus
ctgtggacggtaactaat

SEQ ID NO: 46 - DNA, 19 bp, Campylobacter gracilis
tatagtctcatcccttgcc

SEQ ID NO: 47 - DNA, 17 bp, Campylobacter gracilis
agcaaggggcagattag

SEQ ID NO: 48 - DNA, 19 bp, Campylobacter
  misc_feature: rectus/concisus
ctgttgtcctctagtgtag

SEQ ID NO: 49 - DNA, 20 bp, Campylobacter
  misc_feature: rectus/concisus
tatagcctcatcctacaccg

SEQ ID NO: 50 - DNA, 19 bp, Campylobacter
  misc_feature: cluster (C.rectus/showae/curvus)
gtgcttattccttgggtac

SEQ ID NO: 51 - DNA, 18 bp, Campylobacter
  misc_feature: cluster (C.rectus/showae/curvus)
cggtttggtatttgggct

SEQ ID NO: 52 - DNA, 18 bp, Campylobacter showae
agccctatccattaccga

SEQ ID NO: 53 - DNA, 19 bp, Campylobacter showae
gtaatgggcaagttagcta

SEQ ID NO: 54 - DNA, 18 bp, Capnocytophaga
  misc_feature: sp. _X066
actcaaactcgcgtccaa

SEQ ID NO: 55 - DNA, 18 bp, Capnocytophaga
  misc_feature: sp. _X089
ggtctcagtcactcgaaa

SEQ ID NO: 56 - DNA, 19 bp, Capnocytophaga
  misc_feature: sp. _X089
ttaggtaaccgtcttcagg

SEQ ID NO: 57 - DNA, 18 bp, Capnocytophaga
  misc_feature: sp. AA032
tcaaactacgcgttagcc

SEQ ID NO: 58 - DNA, 18 bp, Capnocytophaga
  misc_feature: sp. AA032
caacagttccaaaggcag

SEQ ID NO: 59 - DNA, 18 bp, Capnocytophaga
  misc_feature: sp. BB167
gccgtgctcttataccat

SEQ ID NO: 60 - DNA, 18 bp, Capnocytophaga
  misc_feature: sp. BB167
aataagtgcacgatgccg

SEQ ID NO: 61 - DNA, 20 bp, Capnocytophaga ochracea
  misc_feature: sp. BB167
attagagggctcgaccttac

SEQ ID NO: 62 - DNA, 18 bp, Capnocytophaga
  misc_feature: Cluster I (Capnocytophaga sp. BM058/BU084/ DZ074)
ctaagtacgtcagtctcc

SEQ ID NO: 63 - DNA, 18 bp, Capnocytophaga
  misc_feature: Cluster II  (C.ochracea/BM058/BU084/DZ074/ BR085)
ctcgctagcaactaatgg

SEQ ID NO: 64 - DNA, 18 bp, Capnocytophaga
  misc_feature: sp. BR085
tatctatcgtcgcctcgg

SEQ ID NO: 65 - DNA, 19 bp, Capnocytophaga
  misc_feature: sp. DS022
gcttattcacagagtaccg

SEQ ID NO: 66 - DNA, 19 bp, Capnocytophaga
  misc_feature: sp. DS022
tctaagtacgtcagtcacc

SEQ ID NO: 67 - DNA, 20 bp, Capnocytophaga gingivalis
gccactcaacttatcatcaa

SEQ ID NO: 68 - DNA, 20 bp, Capnocytophaga sputigena
taaaaacgatgccgctccta

SEQ ID NO: 69 - DNA, 18 bp, Cardiobacterium hominis
aacgtcaattgcgcaggt

SEQ ID NO: 70 - DNA, 18 bp, Corynebacterium durum
agactgcaaacacacagc

SEQ ID NO: 71 - DNA, 18 bp, Corynebacterium matruchotii
ttccagcgcgttgtacta

SEQ ID NO: 72 - DNA, 19 bp, Cryptobacterium curtum
acatctctgtcacttcacc

SEQ ID NO: 73 - DNA, 19 bp, Desulfobulbus
  misc_feature: sp. _R004/CH031
agttattcgctgccttgca

SEQ ID NO: 74 - DNA, 18 bp, Dialister invisus
gcggctcttggaacttat

SEQ ID NO: 75 - DNA, 17 bp, Dialister invisus
tttctgcagatcgcgat

SEQ ID NO: 76 - DNA, 18 bp, Dialister pneumosintes
cctcttaaggcgatagct

SEQ ID NO: 77 - DNA, 18 bp, Dialister pneumosintes
tcaccacaaaccctttcg

SEQ ID NO: 78 - DNA, 18 bp, Eikenella corrodens
aataacgcgaggtcttgc

SEQ ID NO: 79 - DNA, 18 bp, Escherichia coli
agcacattctcatctctg

SEQ ID NO: 80 - DNA, 18 bp, Eubacterium brachy
ctcctaggttactgtcag

SEQ ID NO: 81 - DNA, 19 bp, Eubacterium infirmum
gtccgaagaagaatccgat

SEQ ID NO: 82 - DNA, 19 bp, Eubacterium nodatum
aaccgagctttcagtggga

SEQ ID NO: 83 - DNA, 18 bp, Eubacterium saphenum
ctgtccgaagaaataccc

SEQ ID NO: 84 - DNA, 18 bp, Eubacterium saphenum
cactcaagtctgccagtt

SEQ ID NO: 85 - DNA, 20 bp, Eubacterium
  misc_feature: sp. strain A3MT
gctttgctgtttctatctcc

SEQ ID NO: 86 - DNA, 18 bp, Eubacterium
  misc_feature: sp. BB124
gaagtctcctcggcaata

SEQ ID NO: 87 - DNA, 18 bp, Eubacterium
  misc_feature: sp. BB142
aagctcgtctataaccgc

SEQ ID NO: 88 - DNA, 18 bp, Eubacterium
  misc_feature: sp. DO008
gtcattcttccacccgaa

SEQ ID NO: 89 - DNA, 21 bp, Eubacterium
  misc_feature: sp. IR009
gacttccttttaacagcttcg

SEQ ID NO: 90 - DNA, 19 bp, Eubacterium sulci
gtgtatctcttgcgttatg

SEQ ID NO: 91 - DNA, 18 bp, Eubacterium yurii
cccaacagtagttgagct

SEQ ID NO: 92 - DNA, 18 bp, Eubacterium yurii
cgtgtgtcacaggttgat

SEQ ID NO: 93 - DNA, 17 bp, Filifactor alocis
aggctgtcattggtatg

SEQ ID NO: 94 - DNA, 20 bp, Filifactor alocis
cctgcactcaagttaaacag

SEQ ID NO: 95 - DNA, 18 bp, Fusobacterium
  misc_feature: Cluster (F.nucleatum/naviforme/ss. vincentii/ CZ006/_R002)
cacttcacagctttgcga

SEQ ID NO: 96 - DNA, 18 bp, Fusobacterium nucleatum
cttcccatcattgccttg

SEQ ID NO: 97 - DNA, 18 bp, Fusobacterium nucleatum
  misc_feature: ss. polymorphum
gttgtccctatctgtgag

SEQ ID NO: 98 - DNA, 18 bp, Fusobacterium periodonticum
gtcatgcagtttccaacg

SEQ ID NO: 99 - DNA, 23 bp, Fusobacterium
  misc_feature: sp. _I035
attctaagatgccttataatcat

SEQ ID NO: 100 - DNA, 19 bp, Fusobacterium
  misc_feature: sp. BS011
cataatcttaggatgccct

SEQ ID NO: 101 - DNA, 19 bp, Gemella haemolysans
ggtaccgtctctactgtgt

SEQ ID NO: 102 - DNA, 23 bp, Gemella morbillorum
taaatatctctcatgcgagaaat

SEQ ID NO: 103 - DNA, 18 bp, Granulicatella
  misc_feature: adiacens/elegans
tccatccatcagcagaag

SEQ ID NO: 104 - DNA, 17 bp, Haemophilus influenzae
acgggtgccagagttaa

SEQ ID NO: 105 - DNA, 18 bp, Haemophilus
  misc_feature: parainfluenzae/paraphrophilus
actaaatgccttcctcgc

SEQ ID NO: 106 - DNA, 18 bp, Haemophilus segnis
aaacatcacgccttcctc

SEQ ID NO: 107 - DNA, 18 bp, Haemophilus segnis
ctctctcctgttaccgtt

SEQ ID NO: 108 - DNA, 18 bp, Haemophilus paraphrophaemolyticus
  misc_feature: BJ021
ggattgcttccctctgta

SEQ ID NO: 109 - DNA, 18 bp, Haemophilus
  misc_feature: sp. BJ095
atcgccaccctctgtata

SEQ ID NO: 110 - DNA, 18 bp, Haemophilus
  misc_feature: sp. BJ095
cactccttcctcaatacc

SEQ ID NO: 111 - DNA, 18 bp, Kingella denitrificans
cactactcggtacattcc

SEQ ID NO: 112 - DNA, 17 bp, Kingella denitrificans
ttggctgctcaaatagc

SEQ ID NO: 113 - DNA, 19 bp, Kingella oralis
gcttaccagttcaaaacgc

SEQ ID NO: 114 - DNA, 16 bp, Lactobacillus
  misc_feature: Cluster  (L.casei/rhamnosus/zeae)
cagccaagaaccatgc

SEQ ID NO: 115 - DNA, 18 bp, Lactobacillus
  misc_feature: Cluster  (L.casei/rhamnosus/zeae)
agttactctgccgaccat

SEQ ID NO: 116 - DNA, 19 bp, Lactobacillus fermentum
atcaatcaattgggccaac

SEQ ID NO: 117 - DNA, 19 bp, Lactobacillus fermentum
accgtcaacgtatgaacag

SEQ ID NO: 118 - DNA, 18 bp, Lactobacillus gasseri
ggtgttatcccagtctct

SEQ ID NO: 119 - DNA, 18 bp, Lactobacillus gasseri
ctagacatgcgtctagtg

SEQ ID NO: 120 - DNA, 18 bp, Lactobacillus vaginalis
  misc_feature: CX036
ggcccatcatgaagtgat

SEQ ID NO: 121 - DNA, 18 bp, Lactobacillus vaginalis
  misc_feature: CX036
acagttactctcacgcac

SEQ ID NO: 122 - DNA, 18 bp, Lactobacillus
  misc_feature: sp. HT070
tacgattggcgctagatg

SEQ ID NO: 123 - DNA, 18 bp, Lautropia mirabilis
acccccaaatctcttcag

SEQ ID NO: 124 - DNA, 18 bp, Lautropia mirabilis
tttcgttcccgccaaaag

SEQ ID NO: 125 - DNA, 19 bp, Lautropia
  misc_feature: sp. AP009
tctccaaggttccggacat

SEQ ID NO: 126 - DNA, 18 bp, Leptotrichia buccalis
tgatggcaactagcgata

SEQ ID NO: 127 - DNA, 18 bp, Leptotrichia hofstadii
tatcaggtgttgtcagtc

SEQ ID NO: 128 - DNA, 18 bp, Leptotrichia
  misc_feature: sp. DR011
ggtaactaagcaacaggg

SEQ ID NO: 129 - DNA, 17 bp, Leptotrichia
  misc_feature: sp. FB074/BB002
tatgacctcccggcgat

SEQ ID NO: 130 - DNA, 17 bp, Leptotrichia
  misc_feature: sp. GT018
ccacatagtttccaggg

SEQ ID NO: 131 - DNA, 18 bp, Leptotrichia wadei
ctctagccgcatagtttc

SEQ ID NO: 132 - DNA, 18 bp, Leptotrichia wadei
ctttgaaggttggcttgg

SEQ ID NO: 133 - DNA, 18 bp, Megasphaera
  misc_feature: sp. BB166
cgctaagaggaccgtatt

SEQ ID NO: 134 - DNA, 18 bp, Megasphaera
  misc_feature: sp. BB166
tcttcctcttaccatgcg

SEQ ID NO: 135 - DNA, 19 bp, Megasphaera
  misc_feature: sp. BU057
tctctactccttgcgatca

SEQ ID NO: 136 - DNA, 18 bp, Megasphaera
  misc_feature: sp. BU057
agtcatgcgactttcgga

SEQ ID NO: 137 - DNA, 18 bp, Megasphaera
  misc_feature: sp. CS025
tctacgcccttcactcaa

SEQ ID NO: 138 - DNA, 18 bp, Megasphaera
  misc_feature: sp. CS025
gatactctcagccaacca

SEQ ID NO: 139 - DNA, 18 bp, Micromonas micros
ttctgtggtctcatgcgg

SEQ ID NO: 140 - DNA, 18 bp, Micromonas
  misc_feature: Cluster  (M.micros/FG014/BS044)
catgcgattctgtggtct

SEQ ID NO: 141 - DNA, 19 bp, Micromonas
  misc_feature: sp. DA014
attaatcgcggtttcccac

SEQ ID NO: 142 - DNA, 18 bp, Mycoplasma faucium
ctcccaccgactaatgat

SEQ ID NO: 143 - DNA, 19 bp, Mycoplasma hominis
gtaccgtcagtctgcaatc

SEQ ID NO: 144 - DNA, 18 bp, Mycoplasma salivarium
accgtcaatgtagaagca

SEQ ID NO: 145 - DNA, 18 bp, Neisseria elongata
tccgtctctgaaaggttc

SEQ ID NO: 146 - DNA, 18 bp, Neisseria elongata
ttatgagattggctccgc

SEQ ID NO: 147 - DNA, 18 bp, Neisseria flavescens
cgtcatcagctgtcgata

SEQ ID NO: 148 - DNA, 19 bp, Neisseria
  misc_feature: Cluster I  (N.mucosa/sicca/flava/AP015)
tattaaccccgcccttttc

SEQ ID NO: 149 - DNA, 18 bp, Neisseria pharyngis
gggtattaaccctgtcct

SEQ ID NO: 150 - DNA, 18 bp, Neisseria
  misc_feature: Cluster II  (N.polysaccharea/gonorrhoeae/ meningitidis)
caaccgaatgatggcaac

SEQ ID NO: 151 - DNA, 19 bp, Neisseria
  misc_feature: Cluster II  (N.polysaccharea/gonorrhoeae/ meningitidis)
tttccctctcaagacgtat

SEQ ID NO: 152 - DNA, 18 bp, Neisseria bacilliformis
  misc_feature: /AP132
gaataacgcgaggtccta

SEQ ID NO: 153 - DNA, 16 bp, Neisseria
  misc_feature: Cluster III  (N.elongata/AP015/Eikenella corrodens
gaaggttccgtacatg

SEQ ID NO: 154 - DNA, 17 bp, Neisseria mucosa
  misc_feature: AP060
gtcagcacgcaatggta

SEQ ID NO: 155 - DNA, 18 bp, Neisseria
  misc_feature: sp. B33KA
gtcatcagctcctggtat

SEQ ID NO: 156 - DNA, 17 bp, Olsenella genomospecies
  misc_feature: C1
catctgagtgtcaagcc

SEQ ID NO: 157 - DNA, 18 bp, Olsenella genomospecies
  misc_feature: C1
taactctcgacctactgg

SEQ ID NO: 158 - DNA, 18 bp, Peptostreptococcus
  misc_feature: sp. CK035
attaaacacctgtccgag

SEQ ID NO: 159 - DNA, 18 bp, Porphyromonas catoniae
catttcctctagcctgga

SEQ ID NO: 160 - DNA, 18 bp, Porphyromonas catoniae
ttcaccggtggtcatcta

SEQ ID NO: 161 - DNA, 19 bp, Porphyromonas endodontalis
  misc_feature: Cluster (P. endodontalis/F016/BB134/AJ002)
aagcaaactctcatctgcc

SEQ ID NO: 162 - DNA, 18 bp, Porphyromonas endodontalis
  misc_feature: Cluster  (P. endodontalis/F016/BB134/AJ002)
tccctaattacaggcagg

SEQ ID NO: 163 - DNA, 18 bp, Porphyromonas gingivalis
tcagtcgcagtatggcaa

SEQ ID NO: 164 - DNA, 18 bp, Porphyromonas gingivalis
gtggaagcttgacggtat

SEQ ID NO: 165 - DNA, 18 bp, Porphyromonas
  misc_feature: sp. BB134
ctttccgtctttccccat

SEQ ID NO: 166 - DNA, 18 bp, Porphyromonas
  misc_feature: sp. BB134
tttccgtctttccccatg

SEQ ID NO: 167 - DNA, 18 bp, Porphyromonas
  misc_feature: Cluster (Porphyromonas sp. BR037/DP023/EP003)
atgccctatcccaagtgt

SEQ ID NO: 168 - DNA, 18 bp, Porphyromonas
  misc_feature: sp. CW034/DS033
atgcccaaagtggagtgt

SEQ ID NO: 169 - DNA, 18 bp, Porphyromonas
  misc_feature: sp. CW034/DS033
ttggatgcccaaagtgga

SEQ ID NO: 170 - DNA, 17 bp, Porphyromonas
  misc_feature: sp. DP023
cttggagtaggatgccc

SEQ ID NO: 171 - DNA, 19 bp, Porphyromonas
  misc_feature: sp. DP023
tttccttggagtaggatgc

SEQ ID NO: 172 - DNA, 18 bp, Prevotella buccae
tctctgaatcattctcct

SEQ ID NO: 173 - DNA, 18 bp, Prevotella buccae
cacgtgggcaactttatc

SEQ ID NO: 174 - DNA, 18 bp, Prevotella heparinolytica
caaggcacccagtatcaa

SEQ ID NO: 175 - DNA, 17 bp, Prevotella heparinolytica
ccggataattcggttgc

SEQ ID NO: 176 - DNA, 18 bp, Prevotella intermedia
cgctttactccccaacaa

SEQ ID NO: 177 - DNA, 19 bp, Prevotella loeschii
  misc_feature: GU027
gagcacccggatttcacaa

SEQ ID NO: 178 - DNA, 21 bp, Prevotella
  misc_feature: Cluster  (P.loeschii/GU027/strain B31FD)
atgccaccgactctctgtatg

SEQ ID NO: 179 - DNA, 18 bp, Prevotella melaninogenica
cacacttaatctccagcc

SEQ ID NO: 180 - DNA, 18 bp, Prevotella nigrescens
gctgcgactgcaattcaa

SEQ ID NO: 181 - DNA, 18 bp, Prevotella nigrescens
gtttcatatcggatgccg

SEQ ID NO: 182 - DNA, 18 bp, Prevotella oralis
ttcggactgcatacggaa

SEQ ID NO: 183 - DNA, 18 bp, Prevotella oralis
accgcatttacatacggc

SEQ ID NO: 184 - DNA, 16 bp, Prevotella oris
  misc_feature: F045
tcagtcagaggcaggt

SEQ ID NO: 185 - DNA, 18 bp, Prevotella oulora
agctctcgtcatcgtttc

SEQ ID NO: 186 - DNA, 18 bp, Prevotella oulora
ctctaatgagccaacagc

SEQ ID NO: 187 - DNA, 18 bp, Prevotella pallens
ataacgcacacgtgcatc

SEQ ID NO: 188 - DNA, 18 bp, Prevotella denticola
  misc_feature: sp. AH005/AO036
catcgaagaacatgcggt

SEQ ID NO: 189 - DNA, 18 bp, Prevotella denticola
  misc_feature: sp. AH005
tctgtatcgttctcctgc

SEQ ID NO: 190 - DNA, 18 bp, Prevotella
  misc_feature: sp. AH125
cgcgtgtccctctttatt

SEQ ID NO: 191 - DNA, 18 bp, Prevotella
  misc_feature: sp. AH125
acgcgtgtccctctttat

SEQ ID NO: 192 - DNA, 18 bp, Prevotella
  misc_feature: sp. BE073
ccgtcagtgaagaccata

SEQ ID NO: 193 - DNA, 18 bp, Prevotella
  misc_feature: sp. BI027
ctacctactacgcactca

SEQ ID NO: 194 - DNA, 18 bp, Prevotella
  misc_feature: sp. BI027
gatcgaagtcttggtgag

SEQ ID NO: 195 - DNA, 18 bp, Prevotella
  misc_feature: sp. CY006/FL019
cgttcacccttttatccc

SEQ ID NO: 196 - DNA, 21 bp, Prevotella
  misc_feature: sp. DO022
agaccccgaagggcgtattta

SEQ ID NO: 197 - DNA, 18 bp, Prevotella
  misc_feature: sp. DO027
cccaagcttaacctgatg

SEQ ID NO: 198 - DNA, 19 bp, Prevotella
  misc_feature: sp. DO027
gatgccatcaaaggattac

SEQ ID NO: 199 - DNA, 19 bp, Prevotella
  misc_feature: sp. DO039
gcgagaaagcaacaacatc

SEQ ID NO: 200 - DNA, 17 bp, Prevotella
  misc_feature: sp. DO039
cggcattgaaagcaagc

SEQ ID NO: 201 - DNA, 18 bp, Prevotella
  misc_feature: sp. DO045
acatgcaacctgccttct

SEQ ID NO: 202 - DNA, 18 bp, Prevotella
  misc_feature: sp. DO045
cacaacacgcttagtcca

SEQ ID NO: 203 - DNA, 19 bp, Prevotella
  misc_feature: sp. FM005
cacagtaatgggtaggttg

SEQ ID NO: 204 - DNA, 18 bp, Prevotella
  misc_feature: sp. HF050
ccaagagtgtccgaagaa

SEQ ID NO: 205 - DNA, 18 bp, Prevotella tannerae
ctgcataacagagttggg

SEQ ID NO: 206 - DNA, 18 bp, Prevotella tannerae
cagctgacttatactccc

SEQ ID NO: 207 - DNA, 18 bp, Propionibacterium acnes
tctctgagcactcccgat

SEQ ID NO: 208 - DNA, 20 bp, Propionibacterium
  misc_feature: sp. strain FMA5
tgagcgcttccagtacatgt

SEQ ID NO: 209 - DNA, 18 bp, Pseudomonas aeruginosa
ccactaccaggcagattc

SEQ ID NO: 210 - DNA, 18 bp, Rhodocyclus
  misc_feature: sp. strain A08KA
ccgtattagagagtgcga

SEQ ID NO: 211 - DNA, 19 bp, Rothia dentocariosa
tgacgcagtccagtatatg

SEQ ID NO: 212 - DNA, 18 bp, Rothia dentocariosa
ctgacgcagtccagtata

SEQ ID NO: 213 - DNA, 18 bp, Rothia dentocariosa
cgcagtccagtatatgtc

SEQ ID NO: 214 - DNA, 17 bp, Rothia
  misc_feature: dentocariosa/mucilaginosa
gcggagattggtcgtat

SEQ ID NO: 215 - DNA, 18 bp, Selenomonas dianae
gaggatgctatctctagc

SEQ ID NO: 216 - DNA, 18 bp, Selenomonas flueggii
accgtcattgcatgacac

SEQ ID NO: 217 - DNA, 18 bp, Selenomonas infelix
attcacccttcgcacgtt

SEQ ID NO: 218 - DNA, 20 bp, Selenomonas noxia
gtaccgtcattacctaatac

SEQ ID NO: 219 - DNA, 18 bp, Selenomonas
  misc_feature: sp. AA024
cttatgtacgttcgtccc

SEQ ID NO: 220 - DNA, 20 bp, Selenomonas
  misc_feature: sp. AA024
ggtaccgtcattacataagc

SEQ ID NO: 221 - DNA, 18 bp, Selenomonas
  misc_feature: sp. AH132
tgacactgttcgcatcac

SEQ ID NO: 222 - DNA, 18 bp, Selenomonas
  misc_feature: sp. AJ036
ttgaggttcgctcaacct

SEQ ID NO: 223 - DNA, 17 bp, Selenomonas
  misc_feature: sp. AJ036
tcgaaggaggatgccct

SEQ ID NO: 224 - DNA, 18 bp, Selenomonas
  misc_feature: sp. CI002
ctttcatgcaggggagat

SEQ ID NO: 225 - DNA, 17 bp, Selenomonas
  misc_feature: sp. CI002
ctcccctgcacttcatt

SEQ ID NO: 226 - DNA, 18 bp, Selenomonas
  misc_feature: sp. CS002
ggcgcaacattcggtatt

SEQ ID NO: 227 - DNA, 18 bp, Selenomonas
  misc_feature: sp. CS002
aagatggacttgcatgcc

SEQ ID NO: 228 - DNA, 18 bp, Selenomonas
  misc_feature: sp. CS015
tttcctccccttcccttt

SEQ ID NO: 229 - DNA, 19 bp, Selenomonas
  misc_feature: sp. CS015
gtaccgtcttaaaaagggc

SEQ ID NO: 230 - DNA, 18 bp, Selenomonas
  misc_feature: sp. CS024
gcactatacacgttcgtc

SEQ ID NO: 231 - DNA, 16 bp, Selenomonas
  misc_feature: sp. CS024
agatgcctccctcgca

SEQ ID NO: 232 - DNA, 17 bp, Selenomonas
  misc_feature: sp. DD020
aacccggttttcgtccc

SEQ ID NO: 233 - DNA, 18 bp, Selenomonas
  misc_feature: sp. DM071
cccatgtgagagagttac

SEQ ID NO: 234 - DNA, 18 bp, Selenomonas
  misc_feature: sp. EZ011
aacgcattgcctcgtcta

SEQ ID NO: 235 - DNA, 16 bp, Selenomonas
  misc_feature: sp. DS051
gaagatgcctccccta

SEQ ID NO: 236 - DNA, 18 bp, Selenomonas
  misc_feature: sp. DS051
attcgcactgcgtacgtt

SEQ ID NO: 237 - DNA, 18 bp, Selenomonas
  misc_feature: sp. EW076
ctctgcatgcttcagtca

SEQ ID NO: 238 - DNA, 17 bp, Selenomonas
  misc_feature: sp. EW076
tttgaggttcgcctacc

SEQ ID NO: 239 - DNA, 18 bp, Selenomonas
  misc_feature: sp. EW079/JS031
gtttctgtcccttacagg

SEQ ID NO: 240 - DNA, 18 bp, Selenomonas
  misc_feature: sp. EW084/DS071
ccgtcattacagagcact

SEQ ID NO: 241 - DNA, 16 bp, Selenomonas
  misc_feature: sp. EW084/DS071
cactccgtacgttcgt

SEQ ID NO: 242 - DNA, 18 bp, Selenomonas sputigena
ccgtcacccaaactcaat

SEQ ID NO: 243 - DNA, 18 bp, Selenomonas sputigena
tctcaagctcggttttcg

SEQ ID NO: 244 - DNA, 17 bp, Streptococcus
  misc_feature: (Genus-specific)
agccgtccctttctggt

SEQ ID NO: 245 - DNA, 21 bp, Streptococcus
  misc_feature: anginosus/intermedius
attctcacacttgttcttcct

SEQ ID NO: 246 - DNA, 22 bp, Streptococcus
  misc_feature: anginosus/gordonii
caactcacagtctatggtgtag

SEQ ID NO: 247 - DNA, 22 bp, Streptococcus
  misc_feature: constellatus/intermedius
cagtaaatgttcttatgcggta

SEQ ID NO: 248 - DNA, 17 bp, Streptococcus
  misc_feature: constellatus/intermedius
acatctaccatgcagta

SEQ ID NO: 249 - DNA, 21 bp, Streptococcus cristatus
catgcaatagtcaatgttatg

SEQ ID NO: 250 - DNA, 18 bp, Streptococcus cristatus
ctcatccagaagagcaag

SEQ ID NO: 251 - DNA, 18 bp, Streptococcus
  misc_feature: Cluster I  (S.gordonii/anginosus/mitis)
agtctatggtgtagcaag

SEQ ID NO: 252 - DNA, 20 bp, Streptococcus mitis
  misc_feature: biovar 2
caayaactgctattatgcgg

SEQ ID NO: 253 - DNA, 21 bp, Streptococcus
  misc_feature: Cluster II   (S.mitis/oralis/pneumoniae)
ccttttaagyaaatgtcatgc

SEQ ID NO: 254 - DNA, 18 bp, Streptococcus mutans
tttactccagactttcct

SEQ ID NO: 255 - DNA, 21 bp, Streptococcus parasanguinis
gtcgacttttatgcggtatta

SEQ ID NO: 256 - DNA, 19 bp, Streptococcus parasanguinis
gtcaacatcatgcaatgtc

SEQ ID NO: 257 - DNA, 19 bp, Streptococcus salivarius
gtcatccattgttatgcgg

SEQ ID NO: 258 - DNA, 18 bp, Streptococcus salivarius
gacatgggtcatccattg

SEQ ID NO: 259 - DNA, 19 bp, Streptococcus
  misc_feature: Cluster III  (S.sanguinis/salivarius/mitis/C3)
tgcaactcatccaagaaga

SEQ ID NO: 260 - DNA, 18 bp, Streptococcus australis
atccggaaagagcaagct

SEQ ID NO: 261 - DNA, 22 bp, Streptococcus
  misc_feature: sp. C6/C3/P4/7A
atgcgataatccattttatgcg

SEQ ID NO: 262 - DNA, 20 bp, Streptococcus infantis
  misc_feature: FN042
gcacctttcaagcagctatc

SEQ ID NO: 263 - DNA, 19 bp, Stretococcus sobrinus
gttaactcctcttatgcgg

SEQ ID NO: 264 - DNA, 19 bp, Stretococcus sobrinus
taacatgagttaactcctc

SEQ ID NO: 265 - DNA, 21 bp, Unknown
  All Synergistes (Genus-specific)
tccttttacagctgacttgaa

SEQ ID NO: 266 - DNA, 17 bp, Synergistes
  misc_feature: sp. _D084
acacgagtgcctcctgt

SEQ ID NO: 267 - DNA, 18 bp, Synergistes
  misc_feature: sp. _W028
ggctcctacctcatacat

SEQ ID NO: 268 - DNA, 18 bp, Synergistes
  misc_feature: sp. _W090
cagactcctaccacatac

SEQ ID NO: 269 - DNA, 21 bp, Synergistes
  misc_feature: sp. BB062
cagactcttaccacgtacatg

SEQ ID NO: 270 - DNA, 18 bp, Synergistes
  misc_feature: sp. BH017
tcagtctcagccgcataa

SEQ ID NO: 271 - DNA, 19 bp, Tannerella
  misc_feature: sp. BU063
tctgttgtaggtaggttgc

SEQ ID NO: 272 - DNA, 18 bp, Unknown
  TM7 sp. _I025
cgaacaacaagctatcgg

SEQ ID NO: 273 - DNA, 18 bp, Unknown
  TM7 sp. AH040
ggcactaattggtttccc

SEQ ID NO: 274 - DNA, 17 bp, Unknown
  TM7 sp. BE109
actttggggcatgttcc

SEQ ID NO: 275 - DNA, 18 bp, Unknown
  TM7 sp. BE109/BU080
gagtttgccagttcgaat

SEQ ID NO: 276 - DNA, 18 bp, Unknown
  Treponema 08Apectinovorum
caccactataccattccc

SEQ ID NO: 277 - DNA, 18 bp, Unknown
  Treponema 08Apectinovorum
cgcttcactctgttccaa

SEQ ID NO: 278 - DNA, 18 bp, Treponema
  misc_feature: (Genus-specific)
taacyggcagtaggggtt

SEQ ID NO: 279 - DNA, 19 bp, Treponema denticola
ctaccgtcatcaaagaagc

SEQ ID NO: 280 - DNA, 22 bp, Treponema lecithinolyticum
tacgaacttaagtactatgtca

SEQ ID NO: 281 - DNA, 18 bp, Treponema lecithinolyticum
ttgctcatccgcctacat

SEQ ID NO: 282 - DNA, 18 bp, Treponema medium
cccttatgaagcactgag

SEQ ID NO: 283 - DNA, 19 bp, Treponema socranskii
  misc_feature: all sub-species
ctctatatgatccctcttg

SEQ ID NO: 284 - DNA, 24 bp, Treponema
  misc_feature: sp. AT039
cttatgaaatattgagtgtattcg

SEQ ID NO: 285 - DNA, 19 bp, Treponema vincentii
ctctaagactgtctactag

SEQ ID NO: 286 - DNA, 18 bp, Veillonella dispar
  misc_feature: X042
tgagttcccacccaaagt

SEQ ID NO: 287 - DNA, 18 bp, Veillonella dispar
  misc_feature: X042
tctctggttctgtccatc

SEQ ID NO: 288 - DNA, 18 bp, Veillonella
  misc_feature: (Genus-specific)
aatcccctccttcagtga

SEQ ID NO: 289 - DNA, 18 bp, Veillonella atypica
cctttcatccagtctcga

SEQ ID NO: 290 - DNA, 18 bp, Veillonella atypica
tcgcacaagaaccattcg

SEQ ID NO: 291 - DNA, 17 bp, Veillonella parvula
atctcgcgatctcgctt

SEQ ID NO: 292 - DNA, 18 bp, Veillonella
  misc_feature: sp. AA050/_X042
ttgcaagaaggcctttcg

SEQ ID NO: 293 - DNA, 20 bp, Veillonella
  misc_feature: sp. AA050/_X042
caatccttctcactatttgc

SEQ ID NO: 294 - DNA, 18 bp, Veillonella
  misc_feature: sp. BU083
cacctttcatccatcctc

SEQ ID NO: 295 - DNA, 18 bp, Unknown
  Bacterial Universal
ttcatccatcctcgatgc

SEQ ID NO: 296 - DNA, 43 bp, Actinobacillus actinomycetemcomitans
ggcacaaacccatctctgagttcttcttcggatgtcaagagta

SEQ ID NO: 297 - DNA, 44 bp, Actinobacillus actinomycetemcomitans
tgctattaacacaccaaccttcctcaataccgaaagaactttac

SEQ ID NO: 298 - DNA, 42 bp, Actinobaculum
  misc_feature: sp. EL030
actgcacgtacggagttaagccccgcattttcacagcagacg

SEQ ID NO: 299 - DNA, 41 bp, Actinobaculum
  misc_feature: sp. EL030
ggcttatccaaagaagaaggcagggtactcacgtattactc

SEQ ID NO: 300 - DNA, 42 bp, Actinomyces georgiae
ccaccaacccccatgcgaggaccagtgaatacccggtattag

SEQ ID NO: 301 - DNA, 40 bp, Actinomyces georgiae
tccacccaacacaaagccaggcttcagcgttcgacttgca

SEQ ID NO: 302 - DNA, 40 bp, Actinomyces gerensceriae
aaaaaagccagaaccccagaagcccgttcgacttgcatgt

SEQ ID NO: 303 - DNA, 42 bp, Actinomyces gerensceriae
acccgccatgcgacagacccagaaacatcccgtattagccac

SEQ ID NO: 304 - DNA, 41 bp, Actinomyces naeslundii
cgtctccggagcagaccggcacatgtcaagccttggtaagg

SEQ ID NO: 305 - DNA, 42 bp, Actinomyces naeslundii
cccgttcgccactcatccagaaccagcaagctggctccttca

SEQ ID NO: 306 - DNA, 41 bp, Actinomyces odontolyticus
actaccctcaccacaaccttgagttgcggcttgaccatgag

SEQ ID NO: 307 - DNA, 43 bp, Actinomyces odontolyticus
ttccaccaacccccatgcgaagatcagtgaatatccagtatta

SEQ ID NO: 308 - DNA, 41 bp, Actinomyces
  misc_feature: sp. AP064
cgccactcatccacacccaccacaaaggaaggcgcttcacc

SEQ ID NO: 309 - DNA, 41 bp, Actinomyces
  misc_feature: sp. strain B19SC
cccaccatgcgacagagccagaatatcccgtattaacacca

SEQ ID NO: 310 - DNA, 42 bp, Actinomyces
  misc_feature: sp. strain B19SC
gcgtctccgcgggaatcgccgacatgtcaagccttggtaagg

SEQ ID NO: 311 - DNA, 42 bp, Actinomyces
  misc_feature: sp. strain B27SC
caccagaccccatgcggagaccagtgaatacccggtattagc

SEQ ID NO: 312 - DNA, 38 bp, Actinomyces
  misc_feature: sp. strain B27SC
cacccgttcgccactcacccacccagaaacccagacga

SEQ ID NO: 313 - DNA, 42 bp, Actinomyces
  misc_feature: sp. EP005
tcatccagaaccagcaaaaaccggtcccttcaccgttcgact

SEQ ID NO: 314 - DNA, 42 bp, Actinomyces
  misc_feature: sp. EP011
ccgtcaacccacccacaaaaaaggagcaggcctgcttcactg

SEQ ID NO: 315 - DNA, 40 bp, Actinomyces
  misc_feature: sp. EP011
tgaaccagcccccacaagggaggaaaccccgtctccggag

SEQ ID NO: 316 - DNA, 42 bp, Actinomyces
  misc_feature: sp. EP053
agagattagcttcacttcacagtgtcgcaacccgttgtaccg

SEQ ID NO: 317 - DNA, 42 bp, Actinomyces
  misc_feature: sp. EP053
caaaaacaccaaaaccggtcgaatctttccaaacccacccat

SEQ ID NO: 318 - DNA, 40 bp, Actinomyces israelii
gccacaccatgcggcacagccagaacaccccgtattagcc

SEQ ID NO: 319 - DNA, 41 bp, Actinomyces israelii
aagagcaggaccttcttcaccggcgaagaggttcacaaccc

SEQ ID NO: 320 - DNA, 42 bp, Atopobium parvulum
acggtttcccgtggctatcccaatgaaaggggcaggttgccc

SEQ ID NO: 321 - DNA, 42 bp, Atopobium parvulum
cgagtcttccatgcggaagtctcgaagtattcggtattatca

SEQ ID NO: 322 - DNA, 42 bp, Atopobium rimae
tgtcgtcatgcggccacattgaagtatcgggtattatcctcg

SEQ ID NO: 323 - DNA, 42 bp, Atopobium rimae
tagctgcggcacggaagacgtattctcccacacctagtgtcc

SEQ ID NO: 324 - DNA, 42 bp, Atopobium
  misc_feature: sp. C019
cacttgagtcatgcggctcaagtgaagtatcgggtattatcc

SEQ ID NO: 325 - DNA, 43 bp, Tannerella forsythia
gacgccccgaaggaagaaagctctcactctccgtcgtctacat

SEQ ID NO: 326 - DNA, 42 bp, Tannerella forsythia
gttatccctctgttgcgggcaggttacatacgcgttactcac

SEQ ID NO: 327 - DNA, 42 bp, Bacteroidetes
  misc_feature: sp. _X083
catcttacgatggcagtaccaacagagtacacgactaatttc

SEQ ID NO: 328 - DNA, 42 bp, Bacteroidetes
  misc_feature: sp. _X083
aataagggttgagcccatacatttgacagctgacttaaaaaa

SEQ ID NO: 329 - DNA, 43 bp, Bacteroidetes
  misc_feature: sp. AU126
ttaaaacaaaaaagatgcctcttccgtttactatggggtatta

SEQ ID NO: 330 - DNA, 43 bp, Bacteroidetes
  misc_feature: sp. AU126
gcaataaaacacacgtgtctcactttactcctgtacaaaagaa

SEQ ID NO: 331 - DNA, 42 bp, Bifidobacterium dentium
cgcgaccccatcccataccgacggatctttcccggaaggaca

SEQ ID NO: 332 - DNA, 42 bp, Bifidobacterium
  misc_feature: sp. CX010
gataggacgcgaccccatcatatggcacaaaaagctttccca

SEQ ID NO: 333 - DNA, 42 bp, Bifidobacterium
  misc_feature: sp. CX010
cccgtttccaggagctatccccaaccatacggcaggttagtc

SEQ ID NO: 334 - DNA, 41 bp, Bifidobacterium
  misc_feature: sp. strain A32ED
aagacccgtctccggatcggtcgggaacatgtcaagcccag

SEQ ID NO: 335 - DNA, 44 bp, Brevundimonas diminuta
tttccctgagttattccgaaccaaaaggcacgttcccacgtgtt

SEQ ID NO: 336 - DNA, 44 bp, Brevundimonas diminuta
gccacgtctccgtgggtttccaggcatgtcaaaaggtggtaagg

SEQ ID NO: 337 - DNA, 42 bp, Bulledia extructa
  misc_feature: sequence also found in Solobacterium moorei
tagtccccgtttccagagattatcccagtcttcgtgggtagg

SEQ ID NO: 338 - DNA, 46 bp, Campylobacter concisus
ccgaaaaactttccctatctaacttatgtaagacaggagtatagag

SEQ ID NO: 339 - DNA, 42 bp, Campylobacter concisus
tcgctgattccactgtggacggtaactaatttagtattccgg

SEQ ID NO: 340 - DNA, 43 bp, Campylobacter gracilis
tagctgatacgatatagtctcatcccttgccgaaattctttcc

SEQ ID NO: 341 - DNA, 41 bp, Campylobacter gracilis
ctgttgtcctccagcaaggggcagattagctatatattact

SEQ ID NO: 342 - DNA, 43 bp, Campylobacter rectus
  misc_feature: Sequence also found in Campylobacter concisus
agtcgtttccaactgttgtcctctagtgtagggcagattagct

SEQ ID NO: 343 - DNA, 25 bp, Campylobacter rectus
  misc_feature: Sequence also found in Campylobacter concisus
atatagcctcatcctacaccgaaaa

SEQ ID NO: 344 - DNA, 36 bp, Campylobacter
  misc_feature: cluster (C.rectus/showae/curvus)
cggagttagccggtgcttattccttgggtaccgtca

SEQ ID NO: 345 - DNA, 42 bp, Campylobacter
  misc_feature: cluster (C.rectus/showae/curvus)
gtggaccataaccggtttggtatttgggcttcgagtgaaatc

SEQ ID NO: 346 - DNA, 42 bp, Campylobacter showae
ctgatacgatatagccctatccattaccgaaaaactttcccg

SEQ ID NO: 347 - DNA, 43 bp, Campylobacter showae
tgttgtcccttagtaatgggcaagttagctatatattactcac

SEQ ID NO: 348 - DNA, 42 bp, Capnocytophaga
  misc_feature: sp. _X066
tttcgcttagccactcaaactcgcgtccaaacagctagtatc

SEQ ID NO: 349 - DNA, 42 bp, Capnocytophaga
  misc_feature: sp. _X089
cacccgtacgccggtctcagtcactcgaaagtaacctccccc

SEQ ID NO: 350 - DNA, 43 bp, Capnocytophaga
  misc_feature: sp. _X089
taaacagctcctttaggtaaccgtcttcaggtactcccagctt

SEQ ID NO: 351 - DNA, 41 bp, Capnocytophaga
  misc_feature: sp. AA032
aaggtaccgtcatcaaactacgcgttagcccttattcttcc

SEQ ID NO: 352 - DNA, 42 bp, Capnocytophaga
  misc_feature: sp. AA032
accattcaagaccaacagttccaaaggcagttgctcagttga

SEQ ID NO: 353 - DNA, 42 bp, Capnocytophaga
  misc_feature: sp. BB167
taagtgcacgatgccgtgctcttataccatagggtattaatc

SEQ ID NO: 354 - DNA, 42 bp, Capnocytophaga
  misc_feature: sp. BB167
cgccgtagctttaataagtgcacgatgccgtgctcttatacc

SEQ ID NO: 355 - DNA, 44 bp, Capnocytophaga ochracea
  misc_feature: sp. BB167
accggcagtcccattagagggctcgaccttactcgttagcaact

SEQ ID NO: 356 - DNA, 42 bp, Capnocytophaga
  misc_feature: Cluster I (Capnocytophaga sp. BM058/BU084/ DZ074)
aaatacctatctctaagtacgtcagtctccatttaaaccttg

SEQ ID NO: 357 - DNA, 42 bp, Capnocytophaga
  misc_feature: Cluster II  (C.ochracea/BM058/BU084/DZ074/ BR085)
tgctcgactcgactcgctagcaactaatggcaggggttgcgc

SEQ ID NO: 358 - DNA, 42 bp, Capnocytophaga
  misc_feature: sp. BR085
tctcagaacccctatctatcgtcgcctcggggagccgttacc

SEQ ID NO: 359 - DNA, 43 bp, Capnocytophaga
  misc_feature: sp. DS022
gagttagccgatgcttattcacagagtaccgtcatcaaactac

SEQ ID NO: 360 - DNA, 43 bp, Capnocytophaga
  misc_feature: sp. DS022
gaaatacctatctctaagtacgtcagtcaccatttaaaccttg

SEQ ID NO: 361 - DNA, 44 bp, Capnocytophaga gingivalis
  misc_feature: S3
cactttcgcttagccactcaacttatcatcaaacagctagtatc

SEQ ID NO: 362 - DNA, 44 bp, Capnocytophaga sputigena
gtagctttaatataaaaacgatgccgctcctatataccattagg

SEQ ID NO: 363 - DNA, 42 bp, Cardiobacterium hominis
tcttctgtaggtaacgtcaattgcgcaggtattaactacgca

SEQ ID NO: 364 - DNA, 42 bp, Corynebacterium durum
tagctacggcacagactgcaaacacacagcccacacctagcg

SEQ ID NO: 365 - DNA, 42 bp, Corynebacterium matruchotii
caccctcacaggttccagcgcgttgtactaaccatgtagcat

SEQ ID NO: 366 - DNA, 43 bp, Cryptobacterium curtum
ctttcggctgcgacatctctgtcacttcacctacatgtcaagc

SEQ ID NO: 367 - DNA, 43 bp, Desulfobulbus
  misc_feature: sp. _R004/CH031
gtcaaacaaaacagttattcgctgccttgcacttcttccctct

SEQ ID NO: 368 - DNA, 41 bp, Dialister invisus
ccatgggccatgcggctcttggaacttattcggtattagca

SEQ ID NO: 369 - DNA, 41 bp, Dialister invisus
aagaactccgcatttctgcagatcgcgatcaatgtcaagac

SEQ ID NO: 370 - DNA, 42 bp, Dialister pneumosintes
cagtcgcaaacccctcttaaggcgatagctttcttgtagagg

SEQ ID NO: 371 - DNA, 42 bp, Dialister pneumosintes
gtctatgattattcaccacaaaccctttcgtcccgaatcaca

SEQ ID NO: 372 - DNA, 42 bp, Eikenella corrodens
atcggccgctcgaataacgcgaggtcttgcgatcccccgctt

SEQ ID NO: 373 - DNA, 42 bp, Escherichia coli
tcggttcccggaagcacattctcatctctgaaaacacttccg

SEQ ID NO: 374 - DNA, 42 bp, Eubacterium brachy
agccggggctttctcctaggttactgtcagttttcatcacct

SEQ ID NO: 375 - DNA, 43 bp, Eubacterium infirmum
cctgtctctcttgtccgaagaagaatccgattaaggatctgtc

SEQ ID NO: 376 - DNA, 43 bp, Eubacterium nodatum
gggctcagttttaaccgagctttcagtgggatgtcaagtcctg

SEQ ID NO: 377 - DNA, 42 bp, Eubacterium saphenum
cacctgtctcctctgtccgaagaaatacccgattaagggtac

SEQ ID NO: 378 - DNA, 42 bp, Eubacterium saphenum
tttcctctcttgcactcaagtctgccagttcgcaaggctaac

SEQ ID NO: 379 - DNA, 44 bp, Eubacterium
  misc_feature: sp. strain A3MT
gtgaccgctttcgctttgctgtttctatctcccaatttaaacag

SEQ ID NO: 380 - DNA, 42 bp, Eubacterium
  misc_feature: sp. BB124
tatgatttgaccgaagtctcctcggcaataattctccgttcg

SEQ ID NO: 381 - DNA, 42 bp, Eubacterium
  misc_feature: sp. BB124
ctaatcagacgcaagctcgtctataaccgcctcagctttgat

SEQ ID NO: 382 - DNA, 42 bp, Eubacterium
  misc_feature: sp. DO008
gccactcagtctgtcattcttccacccgaaggtctccaaaga

SEQ ID NO: 383 - DNA, 45 bp, Eubacterium
  misc_feature: sp. IR009
attccatccgaagacttccttttaacagcttcgttcgacttgcat

SEQ ID NO: 384 - DNA, 43 bp, Eubacterium sulci
caagtgatacatgtgtatctcttgcgttatggggtattaatca

SEQ ID NO: 385 - DNA, 41 bp, Eubacterium yurii
ttagtttcaaggcccaacagtagttgagctactgcctttta

SEQ ID NO: 386 - DNA, 42 bp, Eubacterium yurii
ctatcagttatccgtgtgtcacaggttgattacttacgcgtt

SEQ ID NO: 387 - DNA, 41 bp, Filifactor alocis
tcctcgattaaaaggctgtcattggtatgtcaagtttaggt

SEQ ID NO: 388 - DNA, 44 bp, Filifactor alocis
ccactttcctctcctgcactcaagttaaacagttttaatggctt

SEQ ID NO: 389 - DNA, 42 bp, Fusobacterium
  misc_feature: Cluster (F.nucleatum/naviforme/ss. vincentii/ CZ006/_R002)
tgagattagctccacttcacagctttgcgactctctgttcta

SEQ ID NO: 390 - DNA, 42 bp, Fusobacterium nucleatum
  misc_feature: ss. nucleatum
ctctcaggccggcttcccatcattgccttggtgagccgttac

SEQ ID NO: 391 - DNA, 42 bp, Fusobacterium nucleatum
  misc_feature: ss. polymorphum
tcgtttccaaatgttgtccctatctgtgaggcaagttcttta

SEQ ID NO: 392 - DNA, 42 bp, Fusobacterium periodonticum
tccagtactctagtcatgcagtttccaacgcaatacagagtt

SEQ ID NO: 393 - DNA, 47 bp, Fusobacterium
  misc_feature: sp. _I035
atagctttcataattctaagatgccttataatcataatatcaggtat

SEQ ID NO: 394 - DNA, 43 bp, Fusobacterium
  misc_feature: sp. BS011
gtatataactttcataatcttaggatgccctaaaatcataata

SEQ ID NO: 395 - DNA, 43 bp, Gemella haemolysans
gctttctggttaggtaccgtctctactgtgtatagttactaca

SEQ ID NO: 396 - DNA, 47 bp, Gemella morbillorum
ataaccaacttttaaatatctctcatgcgagaaatactgttatccgg

SEQ ID NO: 397 - DNA, 42 bp, Granulicatella adiacens
  misc_feature: sequence also found in Granulicatella elegans
atgcaccgcgggtccatccatcagcagaagccgaagcctctt

SEQ ID NO: 398 - DNA, 41 bp, Haemophilus influenzae
atcacgttagctacgggtgccagagttaaaccccaaccccc

SEQ ID NO: 399 - DNA, 42 bp, Haemophilus parainfluenzae
  misc_feature: Sequence also found in Haemophilus paraphrophilus
ctagtctattaaactaaatgccttcctcgctaccgaaagaac

SEQ ID NO: 400 - DNA, 42 bp, Haemophilus segnis
gtgatgcctattaaacatcacgccttcctcgtcaccgaaaga

SEQ ID NO: 401 - DNA, 42 bp, Haemophilus segnis
caagaaagcaagctctctcctgttaccgttcgacttgcatgt

SEQ ID NO: 402 - DNA, 42 bp, Haemophilus paraphrophaemolyticus
  misc_feature: BJ021
ctccccctcgcaggattgcttccctctgtatacgccattgta

SEQ ID NO: 403 - DNA, 42 bp, Haemophilus
  misc_feature: sp. BJ095
ccacctcgcggcatcgccaccctctgtatacgccattgtagc

SEQ ID NO: 404 - DNA, 42 bp, Haemophilus
  misc_feature: sp. BJ095
gctattaacatacactccttcctcaataccgaaagaacttta

SEQ ID NO: 405 - DNA, 42 bp, Kingella denitrificans
ttggttatcccccactactcggtacattccaatatgttactc

SEQ ID NO: 406 - DNA, 41 bp, Kingella denitrificans
gctaatcagatattggctgctcaaatagcgcaaggtccgaa

SEQ ID NO: 407 - DNA, 43 bp, Kingella oralis
tgacacactctagcttaccagttcaaaacgcagttcccaagtt

SEQ ID NO: 408 - DNA, 40 bp, Lactobacillus
  misc_feature: Cluster  (L.casei/rhamnosus/zeae)
trcgccatctttcagccaagaaccatgcggttcttggaty

SEQ ID NO: 409 - DNA, 42 bp, Lactobacillus
  misc_feature: Cluster  (L.casei/rhamnosus/zeae)
cacgccgacaacagttactctgccgaccattcttctccaaca

SEQ ID NO: 410 - DNA, 43 bp, Lactobacillus fermentum
ggtgcaagcaccatcaatcaattgggccaacgcgttcgactag

SEQ ID NO: 411 - DNA, 43 bp, Lactobacillus fermentum
ttctggttaaataccgtcaacgtatgaacagttactctcatac

SEQ ID NO: 412 - DNA, 42 bp, Lactobacillus gasseri
catctgtttccaggtgttatcccagtctcttgggcaggttac

SEQ ID NO: 413 - DNA, 42 bp, Lactobacillus gasseri
atcttttaaactctagacatgcgtctagtgttgttatccggt

SEQ ID NO: 414 - DNA, 42 bp, Lactobacillus vaginalis
  misc_feature: CX036
taatgcaccgcgggcccatcatgaagtgatagccgaaaccat

SEQ ID NO: 415 - DNA, 42 bp, Lactobacillus vaginalis
  misc_feature: CX036
gtcactgcgtgaacagttactctcacgcacgttcttctccaa

SEQ ID NO: 416 - DNA, 42 bp, Lactobacillus
  misc_feature: sp. HT070
acatcgtatctctacgattggcgctagatgtcaagacctggt

SEQ ID NO: 417 - DNA, 42 bp, Lautropia mirabilis
tctctttcgagcacccccaaatctcttcagggttccggacat

SEQ ID NO: 418 - DNA, 42 bp, Lautropia mirabilis
tggcacagtccttttcgttcccgccaaaagtgctttacaacc

SEQ ID NO: 419 - DNA, 43 bp, Lautropia
  misc_feature: sp. AP009
caccctcgaatctctccaaggttccggacatgtcaagcgtagg

SEQ ID NO: 420 - DNA, 42 bp, Leptotrichia buccalis
tccccaacttaatgatggcaactagcgataggggttgcgctc

SEQ ID NO: 421 - DNA, 42 bp, Leptotrichia hofstadii
tgccggcaactgtatcaggtgttgtcagtcgtttccgtctgt

SEQ ID NO: 422 - DNA, 42 bp, Leptotrichia
  misc_feature: sp. DR011
caacttaatgatggtaactaagcaacaggggttgcgctcgtt

SEQ ID NO: 423 - DNA, 41 bp, Leptotrichia
  misc_feature: sp. FB074/BB002
cagtcatacttctatgacctcccggcgatgtcaaggtctgg

SEQ ID NO: 424 - DNA, 41 bp, Leptotrichia
  misc_feature: sp. GT018
ccagtactctagccacatagtttccagggcaggcttgcggt

SEQ ID NO: 425 - DNA, 42 bp, Leptotrichia wadei
acctctccagtactctagccgcatagtttccagggcaggctt

SEQ ID NO: 426 - DNA, 42 bp, Leptotrichia wadei
aacttggaccggctttgaaggttggcttggcgttgccgcctt

SEQ ID NO: 427 - DNA, 42 bp, Megasphaera
  misc_feature: sp. BB166
cctcttaccatgcgctaagaggaccgtattcggtattagcag

SEQ ID NO: 428 - DNA, 42 bp, Megasphaera
  misc_feature: sp. BB166
gaggccaccctttcttcctcttaccatgcgctaagaggaccg

SEQ ID NO: 429 - DNA, 43 bp, Megasphaera
  misc_feature: sp. BU057
aagaacagagtatctctactccttgcgatcaatgtcaaggctt

SEQ ID NO: 430 - DNA, 42 bp, Megasphaera
  misc_feature: sp. BU057
ctttcttccgacagtcatgcgactttcggaacgtattcggta

SEQ ID NO: 431 - DNA, 42 bp, Megasphaera
  misc_feature: sp. CS025
gaacggcatatctctacgcccttcactcaatgtcaaggcttg

SEQ ID NO: 432 - DNA, 42 bp, Megasphaera
  misc_feature: sp. CS025
gctttcctctccgatactctcagccaaccagtttctctcccc

SEQ ID NO: 433 - DNA, 42 bp, Micromonas micros
tatatcatgcgattctgtggtctcatgcggtattaatcgtcg

SEQ ID NO: 434 - DNA, 42 bp, Micromonas
  misc_feature: Cluster  (M.micros/FG014/BS044)
tgacccctatatcatgcgattctgtggtctcatgcggtatta

SEQ ID NO: 435 - DNA, 43 bp, Micromonas
  misc_feature: sp. DA014
gtcttatgaggtattaatcgcggtttcccacggctatccctct

SEQ ID NO: 436 - DNA, 42 bp, Mycoplasma faucium
ctgcgtcagtggctcccaccgactaatgatcatcgtttacgg

SEQ ID NO: 437 - DNA, 43 bp, Mycoplasma hominis
ctttctgacaaggtaccgtcagtctgcaatcatttcctattgc

SEQ ID NO: 438 - DNA, 42 bp, Mycoplasma salivarium
ttctaacaaggtaccgtcaatgtagaagcatttcctcaacta

SEQ ID NO: 439 - DNA, 42 bp, Neisseria elongata
cgaaggcacccttccgtctctgaaaggttccgtacatgtcaa

SEQ ID NO: 440 - DNA, 42 bp, Neisseria elongata
ctacgatcggttttatgagattggctccgcctcgcggcttgg

SEQ ID NO: 441 - DNA, 42 bp, Neisseria flavescens
ttcttcaggtaccgtcatcagctgtcgatattagcaacagcc

SEQ ID NO: 442 - DNA, 43 bp, Neisseria
  misc_feature: Cluster I  (N.mucosa/sicca/flava/AP015)
atcagacaggggtattaaccccgcccttttcttccctgacaaa

SEQ ID NO: 443 - DNA, 42 bp, Neisseria pharyngis
gtcatcagacaggggtattaaccctgtccttttcttccctga

SEQ ID NO: 444 - DNA, 42 bp, Neisseria
  misc_feature: Cluster II  (N.polysaccharea/gonorrhoeae/ meningitidis)
cattagagtgcccaaccgaatgatggcaactaatgacaaggg

SEQ ID NO: 445 - DNA, 43 bp, Neisseria
  misc_feature: Cluster II  (N.polysaccharea/gonorrhoeae/ meningitidis)
aaggtcccctgctttccctctcaagacgtatgcggtattagct

SEQ ID NO: 446 - DNA, 42 bp, Neisseria bacilliformis
  misc_feature: AP132
catcggccgctcgaataacgcgaggtcctaagatcccccgct

SEQ ID NO: 447 - DNA, 40 bp, Neisseria
  misc_feature: Cluster III  (N.elongata/AP015/Eikenella corrodens
cttccgtctcyggaaggttccgtacatgtcaagaccaggt

SEQ ID NO: 448 - DNA, 41 bp, Neisseria mucosa
  misc_feature: AP060
tcttccggtaccgtcagcacgcaatggtattaacatcgcgc

SEQ ID NO: 449 - DNA, 42 bp, Neisseria
  misc_feature: sp. B33KA
tcttcaggtaccgtcatcagctcctggtattatcaaaagcct

SEQ ID NO: 450 - DNA, 41 bp, Olsenella
  misc_feature: genomospecies C1
tttccgccgcttcatctgagtgtcaagccctggtaaggttc

SEQ ID NO: 451 - DNA, 42 bp, Olsenella
  misc_feature: genomospecies C1
agcccccggatttaactctcgacctactgggcagcctacgcg

SEQ ID NO: 452 - DNA, 42 bp, Peptostreptococcus
  misc_feature: sp. CK035
aggaagggtgtgattaaacacctgtccgagggatgtcaagct

SEQ ID NO: 453 - DNA, 42 bp, Porphyromonas catoniae
  misc_feature (38)..(38): n is a, c, g, or t
cacagcgaatttcatttcctctagcctggatgcccagnctgg

SEQ ID NO: 454 - DNA, 42 bp, Porphyromonas catoniae
  misc_feature (6)..(7): n is a, c, g, or t
gggaanncgactttcaccggtggtcatctacatttcaatccc

SEQ ID NO: 455 - DNA, 31 bp, Porphyromonas endodontalis
  misc_feature: Cluster (P. endodontalis/F016/BB134/AJ002)
tgtcccgaaggaaagcaaactctcatctgcc

SEQ ID NO: 456 - DNA, 42 bp, Porphyromonas endodontalis
  misc_feature: Cluster  (P. endodontalis/F016/BB134/AJ002)
tcaccgggttattccctaattacaggcaggttgcatacgcgt

SEQ ID NO: 457 - DNA, 42 bp, Porphyromonas gingivalis
cgtgcttcagtgtcagtcgcagtatggcaagctgccttcgca

SEQ ID NO: 458 - DNA, 42 bp, Porphyromonas gingivalis
aacgctttcgctgtggaagcttgacggtatatcgcaaactcc

SEQ ID NO: 459 - DNA, 42 bp, Porphyromonas
  misc_feature: sp. BB134
taatcaataaatctttccgtctttccccatgcggaaaaagaa

SEQ ID NO: 460 - DNA, 42 bp, Porphyromonas
  misc_feature: sp. BB134
aatcaataaatctttccgtctttccccatgcggaaaaagaag

SEQ ID NO: 461 - DNA, 42 bp, Porphyromonas
  misc_feature: Cluster (Porphyromonas sp. BR037/DP023/EP003)
ccttggatkaggatgccctatcccaagtgtacgcggtattag

SEQ ID NO: 462 - DNA, 42 bp, Porphyromonas
  misc_feature: sp. CW034/DS033
cctctactytggatgcccaaagtggagtgtacgcggtattag

SEQ ID NO: 463 - DNA, 42 bp, Porphyromonas
  misc_feature: sp. CW034/DS033
atttcctctactttggatgcccaaagtggagtgtacgcggta

SEQ ID NO: 464 - DNA, 41 bp, Porphyromonas
  misc_feature: sp. DP023
gaatttcatttccttggagtaggatgccctatcccaagtgt

SEQ ID NO: 465 - DNA, 43 bp, Porphyromonas
  misc_feature: sp. DP023
cagcgaatttcatttccttggagtaggatgccctatcccaagt

SEQ ID NO: 466 - DNA, 42 bp, Prevotella buccae
gggacttcatcatctctgaatcattctcctgcaattcaagcc

SEQ ID NO: 467 - DNA, 42 bp, Prevotella buccae
  misc_feature (7)..(7): n is a, c, g, or t
tgcaaanacccacacgtgggcaactttatccccgcataaaag

SEQ ID NO: 468 - DNA, 42 bp, Prevotella heparinolytica
  misc_feature: Bacteroides
ctatactgcactcaaggcacccagtatcaactgcaattttaa

SEQ ID NO: 469 - DNA, 41 bp, Prevotella heparinolytica
  misc_feature: Bacteroides
gcttacccgtttccggataattcggttgcaattcaagcccg

SEQ ID NO: 470 - DNA, 42 bp, Prevotella intermedia
ggcacacgtgcccgctttactccccaacaaaagcagtttaca

SEQ ID NO: 471 - DNA, 43 bp, Prevotella loeschii
  misc_feature: GU027
aagccggatgttgagcacccggatttcacaacacgcttaaggc

SEQ ID NO: 472 - DNA, 44 bp, Prevotella
  misc_feature: Cluster  (P.loeschii/GU027/strain B31FD)
gatggcaytcgcatgccaccgactctctgtatgcgccattgtaa

SEQ ID NO: 473 - DNA, 42 bp, Prevotella melaninogenica
tacatttcacaacacacttaatctccagcctacgctcccttt

SEQ ID NO: 474 - DNA, 42 bp, Prevotella nigrescens
acgtctctgtgggctgcgactgcaattcaagcccgggtaagg

SEQ ID NO: 475 - DNA, 42 bp, Prevotella nigrescens
ggaaaacctttggtttcatatcggatgccgtcaatgaaacac

SEQ ID NO: 476 - DNA, 42 bp, Prevotella oralis
ttagatgactgcttcggactgcatacggaattaggccgcctt

SEQ ID NO: 477 - DNA, 42 bp, Prevotella oralis
tttaaggattggaccgcatttacatacggccgactcgctgta

SEQ ID NO: 478 - DNA, 40 bp, Prevotella oris
  misc_feature: F045
acgggttatccctcagtcagaggcaggttggatacgcgtt

SEQ ID NO: 479 - DNA, 42 bp, Prevotella oulora
cctaagagagcaagctctcgtcatcgtttcccctcgacttgc

SEQ ID NO: 480 - DNA, 42 bp, Prevotella oulora
cttggccgctgactctaatgagccaacagcgggcatccagcg

SEQ ID NO: 481 - DNA, 42 bp, Prevotella pallens
acgatacatgcaataacgcacacgtgcatcaaattattctcg

SEQ ID NO: 482 - DNA, 42 bp, Prevotella denticola
  misc_feature: sp. AH005/AO036
atctgatgccgtcatcgaagaacatgcggtattagtctgcct

SEQ ID NO: 483 - DNA, 42 bp, Prevotella denticola
  misc_feature: sp. AH005
gacctcaacatctctgtatcgttctcctgcaattcaagcccg

SEQ ID NO: 484 - DNA, 42 bp, Prevotella
  misc_feature: sp. AH125
tgcaacaggacacgcgtgtccctctttattccccttaaaaag

SEQ ID NO: 485 - DNA, 42 bp, Prevotella
  misc_feature: sp. AH125
atgcaacaggacacgcgtgtccctctttattccccttaaaaa

SEQ ID NO: 486 - DNA, 42 bp, Prevotella
  misc_feature: sp. BE073
tcacatctgatgccgtcagtgaagaccataaggtattagtct

SEQ ID NO: 487 - DNA, 42 bp, Prevotella
  misc_feature: sp. BI027
cacgaattccgcctacctactacgcactcaagttcaacagtt

SEQ ID NO: 488 - DNA, 42 bp, Prevotella
  misc_feature: sp. BI027
agaacccctactgatcgaagtcttggtgagccgttacctcac

SEQ ID NO: 489 - DNA, 42 bp, Prevotella
  misc_feature: sp. CY006/FL019
caaaggagaacacgttcacccttttatccccgtataaaagca

SEQ ID NO: 490 - DNA, 45 bp, Prevotella
  misc_feature: sp. DO022
gcaccttcacagagaccccgaagggcgtatttatctctaaataat

SEQ ID NO: 491 - DNA, 42 bp, Prevotella
  misc_feature: sp. DO027
gtctccagagtgcccaagcttaacctgatggcaactgaagag

SEQ ID NO: 492 - DNA, 43 bp, Prevotella
  misc_feature: sp. DO027
ttatccttatctgatgccatcaaaggattacataaggtattag

SEQ ID NO: 493 - DNA, 43 bp, Prevotella
  misc_feature: sp. DO039
ttactctcccatgcgagaaagcaacaacatcgggtattaatct

SEQ ID NO: 494 - DNA, 41 bp, Prevotella
  misc_feature: sp. DO039
tgcgccggtcgccggcattgaaagcaagctttcaacccgct

SEQ ID NO: 495 - DNA, 42 bp, Prevotella
  misc_feature: sp. DO045
tcgatcgcatttacatgcaacctgccttctgtaccggccatt

SEQ ID NO: 496 - DNA, 42 bp, Prevotella
  misc_feature: sp. DO045
gcatctacatttcacaacacgcttagtccacggcctacgctc

SEQ ID NO: 497 - DNA, 43 bp, Prevotella
  misc_feature: sp. FM005
ggcaggttatcccacagtaatgggtaggttggatacgcgttac

SEQ ID NO: 498 - DNA, 42 bp, Prevotella
  misc_feature: sp. HF050
catgcagcacctccaagagtgtccgaagaaaaatctatctct

SEQ ID NO: 499 - DNA, 42 bp, Prevotella tannerae
atatagtttcaactgcataacagagttgggctctgatattta

SEQ ID NO: 500 - DNA, 42 bp, Prevotella tannerae
tctgatatttaacagctgacttatactcccgcctgcgctccc

SEQ ID NO: 501 - DNA, 42 bp, Prevotella tannerae
gaggcacacccatctctgagcactcccgatccatgtcaaacc

SEQ ID NO: 502 - DNA, 44 bp, Propionibacterium
  misc_feature: sp. strain FMA5
cacgcccatctctgagcgcttccagtacatgtcaaacccaggta

SEQ ID NO: 503 - DNA, 42 bp, Pseudomonas aeruginosa
ggacgttatcccccactaccaggcagattcctaggcattact

SEQ ID NO: 504 - DNA, 42 bp, Rhodocyclus
  misc_feature: sp. strain A08KA
gtcatccacaccccgtattagagagtgcgatttcttcccagc

SEQ ID NO: 505 - DNA, 43 bp, Rothia dentocariosa
aaacgccatctctgacgcagtccagtatatgtcaagccttggt

SEQ ID NO: 506 - DNA, 42 bp, Rothia dentocariosa
gaaacgccatctctgacgcagtccagtatatgtcaagccttg

SEQ ID NO: 507 - DNA, 17 bp, Rothia dentocariosa
ctgctgcctcccgtagg

SEQ ID NO: 508 - DNA, 41 bp, Rothia dentocariosa
  misc_feature: sequence also in Rothia mucilaginosa
ccaacaccccatgcggagattggtcgtatccggtattagac

SEQ ID NO: 509 - DNA, 42 bp, Selenomonas dianae
tgtctccgaagagaggatgctatctctagcactttcagtcaa

SEQ ID NO: 510 - DNA, 42 bp, Selenomonas flueggii
tcctcgacaggtaccgtcattgcatgacactgttcgcatcac

SEQ ID NO: 511 - DNA, 42 bp, Selenomonas infelix
ttgcaaaggattattcacccttcgcacgttcgtccccatcaa

SEQ ID NO: 512 - DNA, 44 bp, Selenomonas noxia
cttcctcgatgggtaccgtcattacctaatactattcgcattag

SEQ ID NO: 513 - DNA, 42 bp, Selenomonas
  misc_feature: sp. AA024
aagcttattcaacttatgtacgttcgtccccttcaacagagc

SEQ ID NO: 514 - DNA, 44 bp, Selenomonas
  misc_feature: sp. AA024
gcttcctcaaaaggtaccgtcattacataagcttattcaactta

SEQ ID NO: 515 - DNA, 42 bp, Selenomonas
  misc_feature: sp. AH132
accgtcattgcatgacactgttcgcatcacgcacgttcgtcc

SEQ ID NO: 516 - DNA, 42 bp, Selenomonas
  misc_feature: sp. AJ036
gggaaacggtttttgaggttcgctcaacctcgcgggttcgct

SEQ ID NO: 517 - DNA, 41 bp, Selenomonas
  misc_feature: sp. AJ036
ggccatctttcatcgaaggaggatgccctccctcgacttca

SEQ ID NO: 518 - DNA, 42 bp, Selenomonas
  misc_feature: sp. CI002
aaaagaggccatctttcatgcaggggagatgcctcccctgca

SEQ ID NO: 519 - DNA, 41 bp, Selenomonas
  misc_feature: sp. CI002
caggggagatgcctcccctgcacttcattcggtattagcat

SEQ ID NO: 520 - DNA, 42 bp, Selenomonas
  misc_feature: sp. CS002
ccatgcgggaggggcgcaacattcggtattagcagccctttc

SEQ ID NO: 521 - DNA, 42 bp, Selenomonas
  misc_feature: sp. CS002
ccccgcgcttttaagatggacttgcatgcccgcctgcgctcc

SEQ ID NO: 522 - DNA, 42 bp, Selenomonas
  misc_feature: sp. CS015
ttaaaaagggcatttcctccccttccctttcgtccccaacaa

SEQ ID NO: 523 - DNA, 43 bp, Selenomonas
  misc_feature: sp. CS015
cttgctcgtcgggtaccgtcttaaaaagggcatttcctcccct

SEQ ID NO: 524 - DNA, 42 bp, Selenomonas
  misc_feature: sp. CS024
atagcactatttgcactatacacgttcgtccccttcaacaga

SEQ ID NO: 525 - DNA, 40 bp, Selenomonas
  misc_feature: sp. CS024
ttcatgcgggggagatgcctccctcgcacaccattcggta

SEQ ID NO: 526 - DNA, 41 bp, Selenomonas
  misc_feature: sp. DD020
tcaatattctcaaacccggttttcgtcccgtgcaacagagc

SEQ ID NO: 527 - DNA, 42 bp, Selenomonas
  misc_feature: sp. DM071
tttagtatctctcccatgtgagagagttacgtcattcggtat

SEQ ID NO: 528 - DNA, 42 bp, Selenomonas
  misc_feature: sp. EZ011
accgtcttagtaaacgcattgcctcgtctaccctttcgtccc

SEQ ID NO: 529 - DNA, 40 bp, Selenomonas
  misc_feature: sp. DS051
ctttcatgtagggaagatgcctcccctacacgccattcgg

SEQ ID NO: 530 - DNA, 42 bp, Selenomonas
  misc_feature: sp. DS051
ttacacagcactattcgcactgcgtacgttcgtccccttcaa

SEQ ID NO: 531 - DNA, 42 bp, Selenomonas
  misc_feature: sp. EW076
agacacatacatctctgcatgcttcagtcaatgtcaaggcct

SEQ ID NO: 532 - DNA, 41 bp, Selenomonas
  misc_feature: sp. EW076
tgggaaacggtttttgaggttcgcctaccctcgcgggttcg

SEQ ID NO: 533 - DNA, 42 bp, Selenomonas
  misc_feature: sp. EW079/JS031
tcaagaacgatagtttctgtcccttacaggggctgagccctt

SEQ ID NO: 534 - DNA, 42 bp, Selenomonas
  misc_feature: sp. EW084/DS071
cctcgayaggtaccgtcattacagagcactattcgcactccg

SEQ ID NO: 535 - DNA, 40 bp, Selenomonas
  misc_feature: sp. EW084/DS071
gagcactattcgcactccgtacgttcgtccccrtcaacag

SEQ ID NO: 536 - DNA, 42 bp, Selenomonas sputigena
cctcgctcggtaccgtcacccaaactcaatattctcaagctc

SEQ ID NO: 537 - DNA, 42 bp, Selenomonas sputigena
caaactcaatattctcaagctcggttttcgtcccgtgcaaca

SEQ ID NO: 538 - DNA, 40 bp, Streptococcus anginosus
  misc_feature: sequence also found in Streptococcus intermedius
atgaactttccattctcacactcgttcttccttaacaaca

SEQ ID NO: 539 - DNA, 46 bp, Streptococcus anginosus
  misc_feature: sequence also found in Streptococcus gordonii
ctcacccgttcgcaactcacagtctatggtgtagcaagctacggta

SEQ ID NO: 540 - DNA, 46 bp, Streptococcus constellatus
  misc_feature: Sequence also found in Streptococcus intermedius
acatctaccatgcagtaaatgttcttatgcggtattagctatcgtt

SEQ ID NO: 541 - DNA, 41 bp, Streptococcus constellatus
  misc_feature: Streptococcus constellatus/intermedius
tgcaccttttaaacatctaccatgcagtaaatgttcttatg

SEQ ID NO: 542 - DNA, 45 bp, Streptococcus cristatus
ttaattgactatcatgcaatagtcaatgttatgcggtattagcta

SEQ ID NO: 543 - DNA, 42 bp, Streptococcus cristatus
acccgttcgcaactcatccagaagagcaagctcctccttcag

SEQ ID NO: 544 - DNA, 42 bp, Streptococcus
  misc_feature: Cluster I  (S.gordonii/anginosus/mitis)
ttcgcaactcacagtctatggtgtagcaagctacggtataaa

SEQ ID NO: 545 - DNA, 44 bp, Streptococcus mitis
  misc_feature: biovar 2
acagttatcatgcaayaactgctattatgcggtattagctatcg

SEQ ID NO: 546 - DNA, 45 bp, Streptococcus
  misc_feature: Cluster II   (S.mitis/oralis/pneumoniae)
gatgcaattgcaccttttaagyaaatgtcatgcaacatctactct

SEQ ID NO: 547 - DNA, 42 bp, Streptococcus mutans
gagccatagccttttactccagactttcctgaccgcctgcgc

SEQ ID NO: 548 - DNA, 45 bp, Streptococcus parasanguinis
acatcatgcaatgtcgacttttatgcggtattagctatcgtttcc

SEQ ID NO: 549 - DNA, 43 bp, Streptococcus parasanguinis
tgcacctttcaagtcaacatcatgcaatgtcgacttttatgcg

SEQ ID NO: 550 - DNA, 43 bp, Streptococcus salivarius
taaatgacatgggtcatccattgttatgcggtattagctatcg

SEQ ID NO: 551 - DNA, 42 bp, Streptococcus salivarius
tttcaaataaatgacatgggtcatccattgttatgcggtatt

SEQ ID NO: 552 - DNA, 43 bp, Streptococcus
  misc_feature: Cluster III  (S.sanguinis/salivarius/mitis/C3)
tactcacccgttcgcaactcatccaagaagagcaagctcctct

SEQ ID NO: 553 - DNA, 42 bp, Streptococcus australis
cgttcgcaactcatccggaaagagcaagctccttccttcagc

SEQ ID NO: 554 - DNA, 45 bp, Streptococcus
  misc_feature: sp. C6/C3/P4/7A
caatggattatcatgcgataatccattttatgcggtattagctat

SEQ ID NO: 555 - DNA, 44 bp, Streptococcus infantis
  misc_feature: FN042
agtggtgcaattgcacctttcaagcagctatcatgcgatatcta

SEQ ID NO: 556 - DNA, 43 bp, Stretococcus sobrinus
cagttaacatgagttaactcctcttatgcggtattagctatcg

SEQ ID NO: 557 - DNA, 43 bp, Stretococcus sobrinus
cttttaaacagttaacatgagttaactcctcttatgcggtatt

SEQ ID NO: 558 - DNA, 41 bp, Synergistes
  misc_feature: sp. _D084
ccagctgataggacacgagtgcctcctgtcgcgcatctctg

SEQ ID NO: 559 - DNA, 42 bp, Synergistes
  misc_feature: sp. _W028
gtcaccgtttccggctcctacctcatacatgtcaaacccagg

SEQ ID NO: 560 - DNA, 42 bp, Synergistes
  misc_feature: sp. _W090
tcgtcgccctttcagactcctaccacatacatgtcaaaccca

SEQ ID NO: 561 - DNA, 45 bp, Synergistes
  misc_feature: sp. BB062
tcatcatcctttcagactcttaccacgtacatgtcaaacccaggt

SEQ ID NO: 562 - DNA, 42 bp, Synergistes
  misc_feature: sp. BH017
tactccagcacctcagtctcagccgcataacacggttaagcc

SEQ ID NO: 563 - DNA, 43 bp, Tannerella
  misc_feature: sp. BU063
ccggattattcttctgttgtaggtaggttgcatacgcgttact

SEQ ID NO: 564 - DNA, 42 bp, Unknown
  TM7 sp. _I025
agtcaagcagttcgaacaacaagctatcggttgagccgatag

SEQ ID NO: 565 - DNA, 42 bp, Unknown
  TM7 sp. AH040
agggttccaaaaggcactaattggtttcccaaaaattccctg

SEQ ID NO: 566 - DNA, 41 bp, Unknown
  TM7 sp. BE109
cagttatccctcactttggggcatgttcccacgcgttactc

SEQ ID NO: 567 - DNA, 42 bp, Unknown
  TM7 sp. BE109/BU080
tctgctactctcgagtttgccagttcgaataatagtctgtat

SEQ ID NO: 568 - DNA, 42 bp, Treponema
  misc_feature: 08Apectinovorum
attacctactgtcacctctataccattccctgtacagtttat

SEQ ID NO: 569 - DNA, 42 bp, Treponema
  misc_feature: 08Apectinovorum
acgtcaccgcttcgcttcactctgttccaaccattgtagcac

SEQ ID NO: 570 - DNA, 43 bp, Treponema denticola
ttattcgcatgactaccgtcatcaaagaagcattccctcttct

SEQ ID NO: 571 - DNA, 46 bp, Treponema lecithinolyticum
catccgtatctctacgaacttaagtactatgtcaaacccaggtaag

SEQ ID NO: 572 - DNA, 42 bp, Treponema lecithinolyticum
ttcacaccaagcttgctcatccgcctacatgccctttacgcc

SEQ ID NO: 573 - DNA, 42 bp, Treponema medium
tttcctcaataccccttatgaagcactgagtgtattcggtat

SEQ ID NO: 574 - DNA, 43 bp, Treponema socranskii
  misc_feature: (all sub-species)
acgctcacatatctctatatgatccctcttgatgtcaaaccca

SEQ ID NO: 575 - DNA, 48 bp, Treponema
  misc_feature: sp. AT039
tcctcaatacttcttatgaaatattgagtgtattcggtattatctgct

SEQ ID NO: 576 - DNA, 43 bp, Treponema vincentii
accaagccttatctctaagactgtctactagatgtcaaaccca

SEQ ID NO: 577 - DNA, 42 bp, Veillonella dispar
  misc_feature: X042
ggcagtctctcatgagttcccacccaaagtgctggcaacata

SEQ ID NO: 578 - DNA, 42 bp, Veillonella dispar
  misc_feature: X042
gaagaggaaccatctctggttctgtccatcaatgtcaagacc

SEQ ID NO: 579 - DNA, 42 bp, Veillonella atypica
aaatagaggccacctttcatccagtctcgatgccgagattgg

SEQ ID NO: 580 - DNA, 42 bp, Veillonella atypica
tcctcgcactattcgcacaagaaccattcgtcccgattaaca

SEQ ID NO: 581 - DNA, 41 bp, Veillonella parvula
cgggtttgctccatctcgcgatctcgcttccgtctattaac

SEQ ID NO: 582 - DNA, 42 bp, Veillonella
  misc_feature: sp. AA050/_X042
ccttctcactatttgcaagaaggcctttcgtcccgattaaca

SEQ ID NO: 583 - DNA, 44 bp, Veillonella
  misc_feature: sp. AA050/_X042
ttccggtaccgtcaatccttctcactatttgcaagaaggccttt

SEQ ID NO: 584 - DNA, 42 bp, Veillonella
  misc_feature: sp. BU083
atacatagaggccacctttcatccatcctcgatgccgaggtt

SEQ ID NO: 585 - DNA, 42 bp, Veillonella
  misc_feature: sp. BU083
tagaggccacctttcatccatcctcgatgccgaggttagatc